(19) United States
(12) Patent Application Publication
(10) Pub. No.: US 2026/0139040 A2
(43) Pub. Date: May 21, 2026

(54) PRECISION ACTIVATED POLYPEPTIDES

(71) Applicants: Carlo Boutton, Ghent-Zwijnaarde (BE); Pieter Deschaght, Ghent-Zwijnaarde (BE); Ellen Foubert, Ghent-Zwijnaarde (BE); Christelle Nonne, Ghent-Zwijnaarde (BE); Annelies Roobrouck, Ghent-Zwijnaarde (BE); Stephanie Staelens, Ghent-Zwijnaarde (BE)

(72) Inventors: Carlo Boutton, Ghent-Zwijnaarde (BE); Pieter Deschaght, Ghent-Zwijnaarde (BE); Ellen Foubert, Ghent-Zwijnaarde (BE); Christelle Nonne, Ghent-Zwijnaarde (BE); Annelies Roobrouck, Ghent-Zwijnaarde (BE); Stephanie Staelens, Ghent-Zwijnaarde (BE)

(73) Assignee: Ablynx N.V., Zwijnaarde (BE)

(21) Appl. No.: 18/963,982

(22) Filed: Nov. 29, 2024

(30) Foreign Application Priority Data
Dec. 1, 2023 (EP) 23213660.6

(51) Int. Cl. C07K 16/18; A61K 39/00; A61P 35/00; C07K 16/28

(52) U.S. Cl. CPC C07K 16/18; A61P 35/00; C07K 16/2809; A61K 2039/505; C07K 2317/14; C07K 2317/31; C07K 2317/565; C07K 2317/567; C07K 2317/734; C07K 2317/92

(57) ABSTRACT

The present technology provides polypeptides comprising a first immunoglobulin single variable domain (ISVD) binding to albumin, a second ISVD capable of binding to both the constant domain of a human T cell receptor (TCR) on a T cell and the constant domain of a non-human primate TCR on a T cell, wherein said first and second ISVD are linked by a protease cleavable linker, and a targeting moiety. The present technology further provides nucleic acids encoding said polypeptides as well as vectors, hosts and methods to produce these polypeptides. Moreover, the present technology relates to methods for treatment making use of the polypeptides according to the present technology.

### REFERENCE TO AN ELECTRONIC SEQUENCE LISTING

The contents of the electronic sequence listing (A084870239US00-SEQ-JRV.xml; Size: 217,975 bytes; and Date of Creation: Nov. 29, 2024) are herein incorporated by reference in its entirety.

### DESCRIPTION

### 1 Field of the Present Technology

The present technology provides polypeptides comprising a first immunoglobulin single variable domain (ISVD) binding to albumin, a second ISVD binding to both the constant domain of a human T cell receptor (TCR) on a T cell and the constant domain of a non-human primate TCR on a T cell, which first and second ISVD are linked by a protease cleavable linker, and a targeting moiety. The present technology further provides nucleic acids encoding said polypeptides as well as vectors, hosts and methods to produce these polypeptides. Moreover, the present technology relates to methods for treatment making use of the polypeptides according to the present technology.

### 2 Technological Background

Antibody therapy is now an important part of the physician's armamentarium to battle diseases and especially cancer. Monoclonal antibodies have been established as a key therapeutic approach for a range of diseases already for several years.

More recently, immunotherapy has emerged as a rapidly growing area of cancer research. Immunotherapy is directing the body's immune surveillance system, and in particular T cells, to cancer cells.

Cytotoxic T cells (CTL) are T lymphocytes that kill cancer cells, cells that are infected (particularly with viruses), or cells that are damaged in other ways. T lymphocytes (also called T cells) express the T cell receptor (TCR) and the CD3 receptor on the cell surface. The αβ TCR-CD3 complex (or “TCR complex”) is composed of six different type I single-spanning transmembrane proteins: the TCRα and TCRβ chains that form the TCR heterodimer responsible for ligand recognition, and the non-covalently associated CD3γ, CD3δ, CD3ε and ζ chains, which bear cytoplasmic sequence motifs that are tyrosine phosphorylated upon receptor activation and recruit a large number of signaling components (Call et al. 2004, Molecular Immunology 40: 1295-1305).

Both α and β chains of the heterodimeric T cell receptor (TCR) consist of a constant domain and a variable domain. T cells are activated upon TCR recognition of cognate peptide presented by self-MHC molecules, with signal transduction initiated by tyrosine phosphorylated CD3 complexes, leading to T cell proliferation and differentiation.

Rather than eliciting specific T cell responses, which rely on expression by cancer cells of MHC molecules and the presence, generation, transport and display of specific peptide antigens, more recent developments have attempted to combine the advantages of immunotherapy with antibody therapy by engaging all T cells of a patient in a polyclonal fashion via recombinant antibody-based technologies. Antibodies that activate T cells are referred to as T cell engagers. Currently, it is common to generate bispecific antibodies that can target both T cells as well as diseased cells. These bispecific antibodies are thus multitargeting molecules that enhance the patient's immune response to diseased cells. For instance, co-engagement of T cell and tumor cell by the bispecific antibody leads to the formation of a cytolytic synapse between the T cell and the tumor cell, that induces T cell activation and results in tumor cell killing.

While the majority of T cell activating bispecific antibodies target the CD3 complex on the T cell, some bispecific binders that target the constant domain of the αβ T cell receptor have been described in WO 2016/180969 A1 and WO 2022/129637 A1.

Currently, only one bispecific antibody, Blinatumomab (a BiTE molecule recognizing CD19 and CD3), is on the market for use in the treatment of cancer. Although this T cell engaging format was approved in December 2014 for second line treatment by the FDA, many hurdles had to be overcome. The first clinical trials of Blinatumomab were prematurely stopped due to neurologic adverse events, cytokine release syndrome (CRS) and infections on the one hand and the absence of objective clinical responses or robust signs of biological activity on the other hand. The safety profile of such T cell engaging formats is thus of considerable concern to physicians and patients.

To minimize the risk for adverse events and systemic side effects there is a need to engineer T cell engagers that are mainly active at the site of the disease.

One strategy to limit target activation in healthy cells that has been applied in recent years is by using a mechanism called conditional activation. Conditional activation refers to the specific activation of a therapeutic compound only when certain conditions are met. In most cases this translates into a therapeutic compound that is only activated in close proximity of the disease site.

For instance, in the context of cancer therapy, a therapeutic compound would only become active when in the presence of a tumor cell. To be more specific, the therapeutic compound would only become active by activation from tumor specific activators, such as tumor proteases.

Hence, a strategy for creating a therapeutic compound that is conditionally activatable is masking. A masked therapeutic compound contains a natural or artificial “mask” that blocks activation of the therapeutic compound by its intended target until the mask is cleaved off. Once the mask is released from the compound, the compound can then bind to its target and efficacy is restored.

One method to release such a mask from the compound is to use so-called protease activatable linkers. Proteases play an essential role in many biological as well as pathological processes by means of a mechanism called proteolysis. Proteolysis entails the selective cleavage of specific substrates. Overexpression of proteases is known to be present in certain diseases, such as cancer, and neurodegenerative, cardiovascular and pulmonary diseases. Therefore, by using protease-activatable drugs to treat these diseases, the drug will mainly be activated at the disease location.

Several activatable antibodies, also known as pro-antibodies, have been generated and tested over the years. However, although these pro-antibodies have great potential, expression of these new formats can be challenging because complex fusion proteins tend to be harder to express. Additionally, undesired immunogenicity of these pro-antibodies can also be an issue in their development.

Another issue that is often experienced with antibodies in general is that, due to their size, there is a chance they may not be able to penetrate diseased tissue effectively. These issues are, logically, also experienced with pro-antibodies.

Therefore, there remains a need for activatable therapeutic compounds with an acceptable safety and toxicity profile that have sufficient potency after activation at the disease site.

### 3 SUMMARY OF THE PRESENT TECHNOLOGY

The inventors have now found that by using an albumin-binding immunoglobulin single variable domain (ISVD) and a T-cell receptor-binding ISVD linked by a linker susceptible to cleavage by a protease, also referred to as a “protease cleavable linker”, combined with a targeting moiety, a potent masked ISVD construct can be provided.

Therefore, in a first aspect, the present technology relates to a polypeptide, comprising
a) a first immunoglobulin single variable domain (ISVD) specifically binding to human serum albumin, wherein said ISVD essentially consists of 4 framework regions (FR1 to FR4 respectively) and 3 complementarity determining regions (CDR1 to CDR3 respectively), wherein
the amino acid sequence of CDR1 (according to Abm) is GFTFRSFGMS (SEQ ID NO: 29), or an amino acid sequence with 4, 3, 2 or 1 amino acid differences with the sequence GFTFRSFGMS;
the amino acid sequence of CDR2 (according to Abm) is SISGSGSDTL (SEQ ID NO: 30), or an amino acid sequence with 4, 3, 2 or 1 amino acid differences with the sequence SISGSGSDTL;
the amino acid sequence of CDR3 (according to Abm) is GGSLSR (SEQ ID NO: 31), or an amino acid sequence with 4, 3, 2 or 1 amino acid differences with the sequence GGSLSR;
b) a second ISVD specifically binding to the constant domain of a human and non-human primate T cell receptor (TCR) present on a T cell, wherein said ISVD essentially consists of 4 framework regions (FR1 to FR4 respectively) and 3 complementarity determining regions (CDR1 to CDR3 respectively), wherein
the amino acid sequence of CDR1 (according to Abm) is WDVHKINFYG (SEQ ID NO: 5), or an amino acid sequence with 4, 3, 2 or 1 amino acid differences with the sequence WDVHKINFYG;
the amino acid sequence of CDR2 (according to Abm) is HISIGDQTD (SEQ ID NO: 3), or an amino acid sequence with 4, 3, 2 or 1 amino acid differences with the sequence HISIGDQTD;
the amino acid sequence of CDR3 (according to Abm) is LSRIWPYDY (SEQ ID NO: 6), or an amino acid sequence with 4, 3, 2 or 1 amino acid differences with the sequence LSRIWPYDY; and
c) a targeting moiety that specifically binds a target antigen on a target cell, wherein said target antigen is different from TCR and serum albumin, and wherein said target cell is different from a T cell,
   wherein the first and second ISVD are linked via a linker that is susceptible to cleavage by a protease, and
   wherein the first ISVD is a C-terminal or N-terminal ISVD.

In another aspect the present technology relates to a composition comprising the polypeptide according to the present technology.

In a further aspect the present technology relates to the polypeptide or composition according to the present technology for use as a medicament.

The present technology further relates to a method of producing the polypeptide according to the present technology.

In other aspects, the present technology relates to a nucleic acid encoding the polypeptide according to the present technology, a vector comprising said nucleic acid, and a non-human host or non-human host cell comprising said nucleic acid or vector.

### 4 BRIEF DESCRIPTION OF THE DRAWINGS

FIG. 1 shows the human T cell mediated killing of HepG2 cells by non-masked ISVD construct T028100022 and activated ISVD constructs T028100011_EK, T028100002_EK and T028100006_EK. The functional effect of an amino acid stretch at the N-terminus of T cell engager ISVD TCR00688 is evaluated.

FIGS. 2A-2B show the human T cell mediated killing by non-masked ISVD constructs T028100027 and T028100022, and masked ISVD constructs T028100016 and T028100006 compared to the activated (enterokinase treated) ISVD constructs (T028100016_EK and T028100006_EK) (FIG. 2A) of SK-BR-3 cells for the HER2 ISVD constructs or (FIG. 2B) of HepG2 cells for the Glypican-3 ISVD constructs.

FIGS. 3A-3B show T cell activation on HCC1954 (FIG. 3A) and HepG2 (FIG. 3B) cells from two donors by masked ISVD constructs (T028100016 and T028100031) compared to the activated (enterokinase treated) ISVD constructs (T028100016_EK and T028100031_EK).

FIGS. 4A-4C show T cell mediated tumor cell killing by uPA protease activated EGFR ISVD constructs on 3 target cell lines from 2 T cell donors: NCI-H292 (FIG. 4A), LS174T (FIG. 4B), and LoVo (FIG. 4C) cells.

FIGS. 5A-5E show T cell mediated tumor cell killing by uPA protease activated HER2 ISVD constructs on 5 target cell lines from 2 T cell donors: HCC1954 (FIG. 5A), BT-474 (FIG. 5B), ZR-75-1 (FIG. 5C), BT-20 (FIG. 5D), and BT-549 (FIG. 5E) cells.

FIGS. 6A-6C show T cell mediated tumor cell killing by PSA protease activated PSMA ISVD constructs on 3 target cell lines from 2 T cell donors (FIG. 6A and FIG. 6C) or from 1 T cell donor (FIG. 6B): HEK293T FOLH1 (FIG. 6A), LNCaP (FIG. 6B), and 22RV1 (FIG. 6C) cells.

FIGS. 7A-7B show PBMC-mediated tumor cell killing by masked and uPA protease activated EGFR ISVD constructs on NCI-H292 (FIG. 7A) and LoVo (FIG. 7B) cells from 4 PBMC donors.

FIGS. 8A-8C show PBMC-mediated tumor cell killing by masked and uPA protease activated HER2 ISVD constructs on HCC1954 (FIG. 8A), ZR-75-1 (FIG. 8B) and BT-549 (FIG. 8C) cells from 4 PBMC donors.

FIGS. 9A-9B show T cell activation (CD69 expression) by the masked EGFR ISVD construct (T028200194) and the uPA protease activated EGFR ISVD construct (T028200192_uPA) on NCI-H292 (FIG. 9A) and LoVo (FIG. 9B) cells. Data was confirmed with 4 PBMC donors for NCI-H292 cells and 3 PBMC donors for LoVo cells.

FIGS. 10A-10C show T cell activation (CD69 expression) by the masked HER2 ISVD construct (T028200154) and the uPA protease activated HER2 ISVD construct (T028200057_uPA) on HCC1954 (FIG. 10A), ZR-75-1 (FIG. 10B) and BT-549 (FIG. 10C) cells. Data is confirmed with 4 PBMC donors.

FIG. 11 shows the MALDI-MSI and 2D-LC/MS workflow used for the evaluation of ex vivo and in vivo protease dependent activation of ISVD constructs.

FIGS. 12A-12B show absolute intensity values for cleaved product 1-122 (corresponding to the uPA and matriptase cleavage site in the protease cleavable linker) for spotted (FIG. 12A) and sprayed (FIG. 12B) compound T028200192 vs. control on different murine tissues.

FIG. 13 shows absolute intensity values for cleaved product 1-122 (corresponding to the uPA and matriptase cleavage site in the protease cleavable linker) for different tissues from mice treated with compound T028200192 and from mice treated with compound T028200194.

FIG. 14 shows the plasma PK profile of different masked HER2 ISVD constructs with protease cleavable linkers (T028200057, T028200085, T028201458), a control ISVD construct (T028200154) and a mimic of a protease pre-activated ISVD construct (T028200056) in CD1 mice.

FIGS. 15A-15C show the blood PK (FIG. 15A), and the specific tumor uptake (FIG. 15B and FIG. 15C) PK and biodistribution analysis of ⁸⁹Zr-masked (T028200323, T028200324) and non-masked (T028200333) HER2 ISVD constructs from an ImmunoPET Imaging experiment in a HCC1954 tumor model.

FIG. 16 shows the plasma PK profile of a masked EGFR ISVD constructs with protease cleavable linker (T028200192), compared to non-masked ISVD construct (T028200198), in presence or absence of human T cells in blood of NCI-H292 tumor bearing mice.

FIGS. 17A-17C show the concentrations of cleaved and uncleaved masked fractions ISVD (black bars measured with the Total PK assay) and the concentrations of masked ISVD (grey bars measured with the Intact PK assay), at the site of an NCI-H292 tumor implanted subcutaneously in NOG mice. The mean % of cleavage of ISVD construct shows the specific in vivo tumor cleavage of T028200192, an EGFR ISVD construct with a protease cleavable linker at the tumor site (FIG. 17A). The concentration of cleaved and activated product from T028200192 increases with dose level at 24 h in the tumor (FIG. 17B) but not in the spleen, used as control tissue (FIG. 17C).

FIGS. 18A-18B show the CD3+ (FIG. 18A) and CD8+ (FIG. 18B) T cell infiltration into NCI-H292 tumors after a single IV administration of an EGFR ISVD construct with a protease cleavable linker to human T cell engrafted NOG mice.

FIG. 19 shows the effect on anti-tumoral growth of the HER2 ISVD construct with a protease cleavable linker, administrated at 1 mg/kg by IV route to human T cell engrafted and HCC1954 bearing NOG mice.

### 5 DETAILED DESCRIPTION OF THE PRESENT TECHNOLOGY

### 5.1 Multispecific Polypeptides

To address the unmet need of therapeutics that can be activated at the site of disease, the present inventors have now discovered that linking an ISVD that binds to human serum albumin with a T cell engaging ISVD through a protease cleavable linker and adding a targeting moiety provides potential for a therapeutic that is active specifically at the area(s) that is/are targeted.

Therefore, in a first aspect the present technology concerns a polypeptide, comprising
a) a first immunoglobulin single variable domain (ISVD) specifically binding to human serum albumin, wherein said ISVD essentially consists of 4 framework regions (FR1 to FR4 respectively) and 3 complementarity determining regions (CDR1 to CDR3 respectively), wherein
the amino acid sequence of CDR1 (according to Abm) is GFTFRSFGMS (SEQ ID NO: 29), or an amino acid sequence with 4, 3, 2 or 1 amino acid differences with the sequence GFTFRSFGMS;
the amino acid sequence of CDR2 (according to Abm) is SISGSGSDTL (SEQ ID NO: 30), or an amino acid sequence with 4, 3, 2 or 1 amino acid differences with the sequence SISGSGSDTL;
the amino acid sequence of CDR3 (according to Abm) is GGSLSR (SEQ ID NO: 31), or an amino acid sequence with 4, 3, 2 or 1 amino acid differences with the sequence GGSLSR;
b) a second ISVD specifically binding to the constant domain of a human and non-human primate T cell receptor (TCR) present on a T cell, wherein said ISVD essentially consists of 4 framework regions (FR1 to FR4 respectively) and 3 complementarity determining regions (CDR1 to CDR3 respectively), wherein
   the amino acid sequence of CDR1 (according to Abm) is WDVHKINFYG (SEQ ID NO: 5), or an amino acid sequence with 4, 3, 2 or 1 amino acid differences with the sequence WDVHKINFYG;
   the amino acid sequence of CDR2 (according to Abm) is HISIGDQTD (SEQ ID NO: 3), or an amino acid sequence with 4, 3, 2 or 1 amino acid differences with the sequence HISIGDQTD;
   the amino acid sequence of CDR3 (according to Abm) is LSRIWPYDY (SEQ ID NO: 6), or an amino acid sequence with 4, 3, 2 or 1 amino acid differences with the sequence LSRIWPYDY; and
c) a targeting moiety that specifically binds a target antigen on a target cell, wherein said target antigen is different from TCR and serum albumin, and wherein said target cell is different from a T cell,
   wherein the first and second ISVD are linked via a linker that is susceptible to cleavage by a protease, and
   wherein the first ISVD is a C-terminal or N-terminal ISVD.

The numbering of the ISVDs (i.e. first ISVD and second ISVD) in the polypeptide can be done starting from the C-terminus of the polypeptide as well as from the N-terminus of the polypeptide.

The C-terminus of a polypeptide, also known as the carboxyl-terminus, is usually defined as the end of an amino acid chain terminated by a free carboxyl group. The C-terminus of an ISVD normally consists of the amino acid sequence VTVSS (SEQ ID NO: 68). The N-terminus, or amino-terminus, is considered the start of the polypeptide, which starts with a free amine group.

Since the second ISVD, binding to the TCR, is linked to the first ISVD this means that the second ISVD is in the second position counting from the C-terminus or the second position counting from the N-terminal respectively. To elaborate further, when the first ISVD is in the first position counting from the C-terminus of the polypeptide, the second ISVD is in the second position counting from the C-terminus of the polypeptide, and when the first ISVD is in the first position counting from the N-terminus of the polypeptide, the second ISVD is in the second position counting from the N-terminus of the polypeptide.

The inventors found that the T cell receptor binding ISVDs with CDRs as disclosed herein have the additional advantage that they show less activity when they are present at the second position in an ISVD construct. Therefore, while the construct is masked by the albumin-binding ISVD, the presence of the TCR binding ISVD in the second position functions as an additional safeguard to inhibit the construct's activity when it is not at the target site.

The inventors found that using a human serum albumin (HSA)-binding ISVD as a masking moiety linked to a T cell receptor (TCR)-binding ISVD by a protease cleavable linker provides the base for a construct that can be activated specifically in the presence of a target through cleavage of the protease cleavable linker present in the ISVD construct.

The HSA-binding ISVD as the masking moiety is most effective when it is at either end position of the ISVD construct. Consequently, the first ISVD (being the HSA-binding ISVD) is either present at the C-terminal or at the N-terminal end of the polypeptide.

Since there is upregulation of protease activity in a lot of diseased tissues, using a protease cleavable linker increases the chances of the polypeptide according to the present technology being active at the site of disease, while in healthy tissue the polypeptide will be in its inactive, masked state. This ensures that the polypeptides according to the present technology will have less off-target activity, while being potent at the site of the targeted disease.

Protease cleavable linkers are well-known in the art and commercially widely available. Protease cleavable linkers have been designed to be targeted by multiple different proteases with which multiple different diseases can be associated. Many studies have also already shown the effectiveness in using protease cleavable linkers to design targeted therapies.

As such, based on the current data, the inventors consider it more than plausible that any protease cleavable linker available should be compatible with the polypeptide of the present technology.

In some embodiments, the protease cleavable linker is cleaved by enterokinase. Tumors originating from enterocytes and goblet cells in the duodenum are known to express enterokinase (Ogata et al. 1992, J. Biol. Chem. 267:3581) Also synthesis of enterokinase by oral squamous cell carcinoma cells, i.e. carcinoma cells outside the duodenum, and its function as activator in complex proteolytic activation cascade has been reported (Vilent et al. 2008, Experimental Cell Research 314:914).

In some embodiments, the protease cleavable linker is cleaved by urokinase (uPA). The elevated expression levels of uPA in breast cancers correspond to the poor prognosis and the metastasis of cancer (Mason et al. 2011, Trends Cell Biol. 21:228; Tang et al. 2013, Biomed. Pharmacother. 67:179) and urokinase (uPA) has been recommended as a diagnostic marker for breast and prostate cancers by the American Society for Clinical Oncology (Duffy et al. 2014, Breast Cancer Res. 16:428) and the German Breast Cancer Society (McCombs et al. 2015, AAPS J. 17:339).

In some embodiments, the protease cleavable linker is cleaved by prostate specific antigen (PSA). A direct correlation between the serum PSA concentration and the clinical stage of the tumor has been described and prostate-specific antigen (PSA) is the most important tumor marker for prostate cancer (Illja et al. 2008, Nature 8:268).

In some embodiments, the protease cleavable linker is cleaved by matriptase. The type II transmembrane serine protease (TTSP), matriptase, has been implicated in breast cancer since it was first discovered in breast cancer cell lines, and is highly expressed by the malignant cells in human breast carcinomas (Bhatt et al. 2003, Biol. Chem. 384:257; Lin et al. 1997, J. Biol. Chem. 272:9147; Oberst et al. 2001, Am. J. Pathol. 158:1301; Jin et al. Histol. Histopathol. 22:305; Bergum et al. 2012, J. Cell Physiol. 227:1604).

Examples of protease cleavable linkers that are usable in constructs according to the present technology can for instance be found in WO 2015/116933, WO 2015/048329, WO 2016/118629, WO 2016/077505, WO 2018/136725, WO 2020/118109, WO 2022/035866.

Protease cleavable linkers have been applied to many different formats as is also illustrated in for instance WO 2009/025846, WO 2016/046778, WO 2018/085555, WO 2019/246392, WO 2019/222282, WO 2019/222283 WO 2019/222283, and WO 2019/222294. In an embodiment the linker susceptible to cleavage by a protease is selected from SEQ ID NO: 52-SEQ ID NO: 55.

In an embodiment, the second ISVD of the polypeptide has a CDR1 with amino acid sequence WDVHKINFYG (SEQ ID NO: 5), or an amino acid sequence with 2 or 1 amino acid differences with the sequence WDVHKINFYG, wherein the amino acid differences are selected from:
W to G
D to Y.

In this embodiment, in the second ISVD of the polypeptide as defined above, the W at position 26 has been substituted by G (W26G) and/or the D at position 27 has been substituted by Y (D27Y), wherein the positions are determined according to Kabat.

In another embodiment, the second ISVD of the polypeptide has a CDR3 with amino acid sequence LSRIWPYDY (SEQ ID NO: 6), or an amino acid sequence with 1 amino acid difference with the sequence LSRIWPYDY, wherein the amino acid difference is selected from:
W to Y.

In this embodiment, in the second ISVD of the polypeptide as defined above, the W at position 99 has been substituted by Y (W99Y), wherein the positions are determined according to Kabat.

In a further embodiment, the second ISVD of the polypeptide has a CDR1 with amino acid sequence WDVHKINFYG (SEQ ID NO: 5), a CDR2 with amino acid sequence HISIGDQTD (SEQ ID NO: 3), and a CDR3 with amino acid sequence LSRIWPYDY (SEQ ID NO: 6).

In an embodiment, the first ISVD of the polypeptide has a CDR1 with amino acid sequence GFTFRSFGMS (SEQ ID NO: 29), a CDR2 with amino acid sequence SISGSGSDTL (SEQ ID NO: 30), and a CDR3 with amino acid sequence GGSLSR (SEQ ID NO: 31).

The inventors have shown that the polypeptides according to the present technology are successfully masked by the HSA-binding ISVD and will not become active when it is not in the presence of the proteases capable of cleaving the linker between the first ISVD and the second ISVD, and the target of the targeting ISVD. Activity increases significantly upon cleavage of the protease cleavable linker, showing that the ISVD constructs' potency can successfully be restored when the conditions of activation are met.

As is commonly known, protease activities are upregulated in a lot of different diseases. In normal, healthy tissues, the expression of proteases is usually low. This means that even if the targeting ISVD binds to a target on a healthy cell, the construct will not be activated because the albumin-binding ISVD is serving as a mask for the TCR-binding ISVD. Additionally, since the inventors selected TCR-binding ISVDs that are less active when in the second position in a construct, there is a decreased risk of the TCR-binding ISVD recruiting T cells even when the albumin-binding ISVD is still linked to the TCR-binding ISVD.

Consequently, the polypeptides according to the present technology, ensure that the polypeptide will only become active once the linker has been cleaved by its respective protease and the targeting ISVD has bound its target.

In an embodiment, the first ISVD and/or the second ISVD is a heavy-chain ISVD. In certain embodiments, the first ISVD and/or the second ISVD is selected from a VHH, a humanized VHH, a (single) domain antibody, a dAb, and a camelized VH.

The smaller size of heavy-chain ISVDs gives them advantages over conventional monoclonal antibodies. Additionally, since they can be produced in vitro, the production process is simple and cost-effective.

In an embodiment, the first ISVD has at least 80%, preferably at least 90%, more preferably at least 95% sequence identity with the amino acid sequence of SEQ ID NO: 32, in which for the purposes of determining the degree of sequence identity, the amino acid residues that form the CDR sequences are disregarded. In certain embodiments the amino acid sequence of the first ISVD comprises or consists of SEQ ID NO: 32.

In a further embodiment, the second ISVD has at least 80%, preferably at least 90%, more preferably at least 95% sequence identity with the amino acid sequence of SEQ ID NO: 1, in which for the purposes of determining the degree of sequence identity, the amino acid residues that form the CDR sequences are disregarded. In certain embodiments the amino acid sequence of the second ISVD comprises or consists of SEQ ID NO: 1.

Another embodiment concerns the polypeptide according to the present technology, wherein the targeting moiety is an ISVD. In an embodiment, the targeting moiety is selected from a VHH, a humanized VHH, a (single) domain antibody, a dAb, and a camelized VH.

In another embodiment, the targeting moiety specifically binds a tumor associated antigen or a tumor antigen.

The term “tumor antigen” as used herein may be understood as those antigens that are presented on tumor cells. These antigens can be presented on the cell surface with an extracellular part, which is often combined with a transmembrane and cytoplasmic part of the molecule. These antigens can sometimes be presented only by tumor cells and never by a normal or healthy cell. Tumor antigens can be exclusively expressed on tumor cells or might represent a tumor specific mutation compared to normal cells. In this case, they are called tumor-specific antigens. However, this will not be the case generally. More common are antigens that are presented by tumor cells and normal cells, and they are called “tumor-associated antigens (TAA)”. These tumor-associated antigens can be overexpressed on tumor cells compared to normal cells or are better accessible for antibody binding in tumor cells due to the less compact structure of the tumor tissue compared to normal tissue. TAA are preferably antigens that are expressed on cells of particular tumors, but that are preferably not expressed in normal cells. Often, TAA are antigens that are normally expressed in cells only at particular points in an organism's development (such as during fetal development) and that are being inappropriately expressed in the organism at the present point of development, or are antigens not expressed in normal tissues or cells of an organ now expressing the antigen.

The polypeptide according to the present technology may further comprise non-cleavable linkers, for instance between the second ISVD and the targeting moiety, such as the ones described by, but not limited to, the linkers in Table A-0.

TABLE A-0
Linker sequences (″ID″ refers to the SEQ ID NO as used herein)

| Name | ID | Amino acid sequence |
|---|---|---|
| 3A linker | 98 | AAA |
| 5GS linker | 99 | GGGGS |
| 7GS linker | 100 | SGGSGGS |
| 8GS linker | 101 | GGGGSGGS |
| 9GS linker | 102 | GGGGSGGGS |
| 10GS linker | 103 | GGGGSGGGGS |
| 15GS linker | 104 | GGGGSGGGGSGGGGS |
| 18GS linker | 105 | GGGGSGGGGSGGGGSGGS |
| 20GS linker | 106 | GGGGSGGGGSGGGGSGGGGS |
| 25GS linker | 107 | GGGGSGGGGSGGGGSGGGGSGGGGS |
| 30GS linker | 108 | GGGGSGGGGSGGGGSGGGGSGGGGSGGGGS |
| 35GS linker | 109 | GGGGSGGGGSGGGGSGGGGSGGGGSGGGGSGGGGS |
| 40GS linker | 110 | GGGGSGGGGSGGGGSGGGGSGGGGSGGGGGGGGSGGGGS |
| G1 hinge | 111 | EPKSCDKTHTCPPCP |
| 9GS-G1 hinge | 112 | GGGGSGGGSEPKSCDKTHTCPPCP |
| Llama upper long | 113 | EPKTPKPQPAAA |
| hinge region | | |
| G3 hinge | 114 | ELKTPLGDTTHTCPRCPEPKSCDTPPPCPRCPEPKSCDTPPPCPRCPEPK |
| | | SCDTPPPCPRCP |

The target cell bound by the polypeptides of the present technology relates in particular to mammalian cells, preferably to primate cells, and even more preferably to human cells. The target cell is preferably a hyperproliferative cell such as e.g., a cancer cell. A target that could be bound by the polypeptides of the present technology on a tumor cell may for instance be a tumor antigen or a tumor-associated antigen. Some examples of tumor antigens or tumor-associated antigens that may be targeted include human epidermal growth factor receptor 2 (HER2), epidermal growth factor receptor (EGFR), Glypican-3 (GPC3), Prostate-specific membrane antigen (PSMA), and others.

In another embodiment, the multispecific-multivalent polypeptide exhibits reduced binding by pre-existing antibodies in human serum. To this end, in one embodiment, one or more ISVDs in the polypeptide exhibit a valine (V) at amino acid position 11 and a leucine (L) at amino acid position 89 (according to Kabat numbering), preferably the ISVD at the C-terminal end of the polypeptide, but preferably each ISVD.

In another embodiment, the polypeptide exhibits an extension of 1 to 5 (preferably naturally occurring) amino acids, such as a single alanine (A) extension, at the C-terminus of the C-terminal ISVD. The C-terminus of an ISVD normally has the sequence VTVSS (SEQ ID NO:68).

In another embodiment, the ISVD exhibits a lysine (K) or glutamine (Q) at position 112 (according to Kabat numbering) in at least on ISVD. In these embodiments, the C-terminus of the ISVD may have the sequence VKVSS (SEQ ID NO: 69), VQVSS (SEQ ID NO: 70), VTVKS (SEQ ID NO: 71), VTVQS (SEQ ID NO: 72), VKVKS (SEQ ID NO: 73), VKVQS (SEQ ID NO: 74), VQVKS (SEQ ID NO: 75), or VQVQS (SEQ ID NO: 76) such that after addition of a single alanine at the C-terminus of the C-terminal ISVD, the C-terminus of the polypeptide for example exhibits the sequence VTVSSA (SEQ ID NO: 77), VKVSSA (SEQ ID NO: 78), VQVSSA (SEQ ID NO: 79), VTVKSA (SEQ ID NO: 80), VTVQSA (SEQ ID NO: 81), VKVKSA (SEQ ID NO: 82), VKVQSA (SEQ ID NO: 83), VQVKSA (SEQ ID NO: 84), or VQVQSA (SEQ ID NO: 85), preferably VTVSSA.

In another embodiment, the polypeptide exhibits a valine (V) at amino acid position 11 and a leucine (L) at amino acid position 89 (according to Kabat numbering) in at least the C-terminal ISVD, optionally a lysine (K) or glutamine (Q) at position 110 (according to Kabat numbering) in at least one ISVD, and exhibits an extension of 1 to 5 (preferably naturally occurring) amino acids, such as a single alanine (A) extension, at the C-terminus of the C-terminal ISVD (such that the C-terminus of the polypeptide for example consists of the sequence VTVSSA, VKVSSA or VQVSSA, preferably VTVSSA). See e.g. WO2012/175741 and WO2015/173325 for further information in this regard.

### 5.2 Immunoglobulin Single Variable Domains

The term “immunoglobulin single variable domain” (ISVD), defines immunoglobulin molecules wherein the antigen binding site is present on, and formed by, a single immunoglobulin domain. This sets immunoglobulin single variable domains apart from “conventional” immunoglobulins (e.g. monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can for example be a heavy chain ISVD, such as a V_{H}, V_{HH}, including a camelized V_{H} or humanized V_{HH}. Preferably, it is a V_{HH}, including a camelized V_{H} or humanized V_{HH}. Heavy chain ISVDs can be derived from a conventional four-chain antibody or from a heavy chain antibody.

For example, the immunoglobulin single variable domain may be a (single) domain antibody (or an amino acid sequence that is suitable for use as a single domain antibody), a “dAb” or dAb (or an amino acid sequence that is suitable for use as a dAb); other single variable domains, or any suitable fragment of any one thereof.

In particular, the immunoglobulin single variable domain may be a NANOBODY® immunoglobulin single variable domain (such as a V_{HH}, including a humanized V_{HH}, or camelized V_{H}) or a suitable fragment thereof. NANOBODY® and NANOBODIES® are registered trademarks of Ablynx N.V.

“V_{HH} domains”, also known as V_{HH}s, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. 1993, Nature 363: 446-448). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'s, reference is made to the review article by Muyldermans 2001 (Reviews in Molecular Biotechnology 74: 277-302).

Typically, the generation of immunoglobulins involves the immunization of experimental animals, fusion of immunoglobulin producing cells to create hybridomas and screening for the desired specificities. Alternatively, immunoglobulins can be generated by screening of naïve or synthetic libraries e.g. by phage display.

The generation of immunoglobulin sequences has been described extensively in various publications, among which WO 94/04678, Hamers-Casterman et al. 1993 and Muyldermans et al. 2001 can be exemplified. In these methods, camelids are immunized with the target antigen in order to induce an immune response against said target antigen. The repertoire of VHHs obtained from said immunization is further screened for VHHs that bind the target antigen.

In these instances, the generation of immunoglobulins requires purified antigen for immunization and/or screening. Antigens can be purified from natural sources, or in the course of recombinant production.

Immunization and/or screening for immunoglobulin sequences can be performed using peptide fragments of such antigens.

The present technology may use immunoglobulin sequences of different origin, comprising mouse, rat, rabbit, donkey, human and camelid immunoglobulin sequences. The technology also includes fully human, humanized, or chimeric sequences. For example, the technology comprises camelid immunoglobulin sequences and humanized camelid immunoglobulin sequences, or camelized domain antibodies, e.g. camelized dAb as described by Ward et al. 1989 (Nature 341:544); see for example WO 94/04678 and Davies and Riechmann (1994, FEBS Letters 339:285 and 1996, Protein Engineering 9:531). Moreover, the technology also uses fused immunoglobulin sequences, e.g. forming a multivalent and/or multispecific construct (for multivalent and multispecific polypeptides containing one or more V_{HH} domains and their preparation, reference is also made to Conrath et al. 2001, J. Biol. Chem. 276 (10): 7346-7350, as well as to for example WO 96/34103 and WO 99/23221), and immunoglobulin sequences comprising tags or other functional moieties, e.g. toxins, labels, radiochemicals, etc., which are derivable from the immunoglobulin sequences of the present technology.

A “humanized V_{HH}” comprises an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example based on the further description herein and the prior art (e.g. WO 2008/020079). Again, it should be noted that such humanized V_{HH}s can be obtained in any suitable manner known per se and thus are not strictly limited to polypeptides that have been obtained using a polypeptide that comprises a naturally occurring V_{HH} domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example based on the further description herein and the prior art (e.g. WO 2008/020079). Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann 1994 and 1996, supra). Preferably, the V_{H} sequence that is used as a starting material or starting point for generating or designing the camelized V_{H} is preferably a V_{H} sequence from a mammal, more preferably the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

A preferred structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

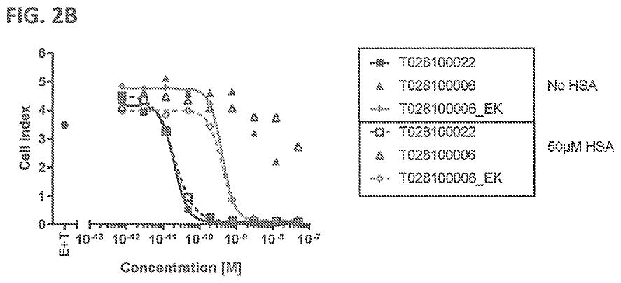

As further described in paragraph q) on pages 58 and 59 of WO 08/020079, the amino acid residues of an immunoglobulin single variable domain can be numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans 2000 (J. Immunol. Methods 240 (1-2): 185-195; see for example FIG. 2 of this publication). It should be noted that—as is well known in the art for V_{H} domains and for V_{HH} domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In the present application, unless indicated otherwise, CDR sequences were determined according to the AbM definition as described in Martin 2010 (In: Kontermann and Dübel (Eds.) 2010, Antibody Engineering, vol 2, Springer Verlag Heidelberg Berlin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

Determination of CDR regions may also be done according to different methods, such as according to Kabat (Martin 2010, In: Kontermann and Dübel (eds.), Antibody Engineering Vol. 2, Springer Verlag Heidelberg Berlin, Chapter 3, pp. 33-51). According to this method, FR1 of an immunoglobulin single variable domain comprises the amino acid residues at positions 1-30, CDR1 of an immunoglobulin single variable domain comprises the amino acid residues at positions 31-35, FR2 of an immunoglobulin single variable domain comprises the amino acids at positions 36-49, CDR2 of an immunoglobulin single variable domain comprises the amino acid residues at positions 50-65, FR3 of an immunoglobulin single variable domain comprises the amino acid residues at positions 66-94, CDR3 of an immunoglobulin single variable domain comprises the amino acid residues at positions 95-102, and FR4 of an immunoglobulin single variable domain comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence or V_{HH} sequence). In one particularly preferred aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISVD sequence used in the technology may contain one or more of hallmark residues (as defined herein), such that the ISVD sequence is a V_{HH}, including a humanized V_{HH} or camelized V_{H}. Some preferred, but non-limiting examples of (suitable combinations of) such framework sequences will become clear from the further disclosure herein.

However, it should be noted that the technology is not limited as to the origin of the ISVD sequence (or of the nucleotide sequence used to express it), nor as to the way that the ISVD sequence or nucleotide sequence is (or has been) generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

For a general description of ISVDs, reference is made to the further description below, as well as to the prior art cited herein. In this respect, it should however be noted that this description and the prior art mainly described ISVDs of the so-called “V_{H}3 class” (i.e. ISVDs with a high degree of sequence homology to human germline sequences of the V_{H}3 class such as DP-47, DP-51, or DP-29). It should however be noted that the technology in its broadest sense can generally use any type of ISVD, and for example also uses the ISVDs belonging to the so-called “V_{H}4 class” (i.e. ISVDs with a high degree of sequence homology to human germline sequences of the V_{H}4 class such as DP-78), as for example described in WO 2007/118670.

Generally, ISVDs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, an ISVD can be defined as an immunoglobulin sequence with the (general) structure

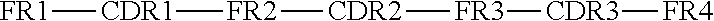

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, an ISVD can be an immunoglobulin sequence with the (general) structure

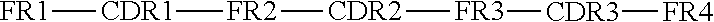

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, an ISVD can be an immunoglobulin sequence with the (general) structure

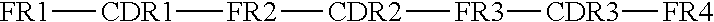

in which FR1 to FR4 refer to framework regions 1 to 4, respectively, and in which CDR1 to CDR3 refer to the complementarity determining regions 1 to 3, respectively, and in which one or more of the amino acid residues at positions 11, 37, 44, 45, 47, 83, 84, 103, 104 and 108 according to the Kabat numbering are selected from the Hallmark residues mentioned in Table A-1 below.

TABLE A-1
Hallmark Residues in ISVDs

| Position | Human V_{H}3 | Hallmark Residues |
|---|---|---|
| 11 | L, V; | L, S, V, M, W, F, T, Q, E, A, R, |
| | predominantly L | G, K, Y, N, P, I; preferably L |
| 37 | V, I, F; | F⁽¹⁾, Y, V, L, A, H, S, I, W, C, |
| | usually V | N, G, D, T, P, preferably F⁽¹⁾ or Y |
| 44⁽⁸⁾ | G | E⁽³⁾, Q⁽³⁾, G⁽²⁾, D, A, K, R, L, P, |
| | | S, V, H, T, N, W, M, I; preferably G⁽²⁾, |
| | | E⁽³⁾ or Q⁽³⁾; most preferably G⁽²⁾ or Q⁽³⁾. |
| 45⁽⁸⁾ | L | L⁽²⁾, R⁽³⁾, P, H, F, G, Q, S, E, T, |
| | | Y, C, I, D, V; preferably L⁽²⁾ or R⁽³⁾ |
| 47⁽⁸⁾ | W, Y | F⁽¹⁾, L⁽¹⁾ or W⁽²⁾ G, I, S, A, V, M, |
| | | R, Y, E, P, T, C, H, K, Q, N, |
| | | D; preferably W⁽²⁾, L⁽¹⁾ or F⁽¹⁾ |
| 83 | R or K; | R, K⁽⁵⁾, T, E⁽⁵⁾, Q, N, S, I, V, G, |
| | usually R | M, L, A, D, Y, H; preferably Kor |
| | | R; most preferably K |
| 84 | A, T, D; | P⁽⁵⁾, S, H, L, A, V, I, T, F, D, R, |
| | predominantly A | Y, N, Q, G, E; preferably P |
| 103 | W | W⁽⁴⁾, R⁽⁶⁾, G, S, K, A, M, Y, L, F, |
| | | T, N, V, Q, P⁽⁶⁾, E, C; preferably W |
| 104 | G | G, A, S, T, D, P, N, E, C, L; |
| | | preferably G |
| 108 | L, M or T; | Q, L⁽⁷⁾, R, P, E, K, S, T, M, A, H; |
| | predominantly L | preferably Q or L⁽⁷⁾ |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q in (non-humanized) V_{HH} sequences that also contain a W at 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

The terms “specificity”, “binding specifically” or “specific binding” refer to the number of different target molecules, such as antigens, from the same organism to which a particular binding unit, such as an ISVD, can bind with sufficiently high affinity (see below). “Specificity”, “binding specifically” or “specific binding” are used interchangeably herein with “selectivity”, “binding selectively” or “selective binding”. Binding units, such as ISVDs, preferably specifically bind to their designated targets.

The specificity/selectivity of a binding unit can be determined based on affinity. The affinity denotes the strength or stability of a molecular interaction. The affinity is commonly given as by the K_{D}, or dissociation constant, which is expressed in units of mol/liter (or M). The affinity can also be expressed as an association constant, K_{A}, which equals 1/K_{D} and is expressed in units of (mol/liter)⁻¹ (or M⁻¹).

The affinity is a measure for the binding strength between a moiety and a binding site on the target molecule: the lower the value of the K_{D}, the stronger the binding strength between a target molecule and a targeting moiety.

Typically, binding units used in the present technology (such as ISVDs) will bind to their targets with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles).

Any K_{D} value greater than 10⁻⁴ mol/liter (or any K_{A} value lower than 10⁴ liters/mol) is generally considered to indicate non-specific binding.

The K_{D} for biological interactions, such as the binding of immunoglobulin sequences to an antigen, which are considered specific are typically in the range of 10⁻⁵ moles/liter (10000 nM or 10 μM) to 10⁻¹² moles/liter (0.001 nM or 1 pM) or less.

Accordingly, specific/selective binding may mean that—using the same measurement method, e.g. SPR—a binding unit (or polypeptide comprising the same) binds to TCR with a K_{D} value of 10⁻⁵ to 10⁻¹² moles/liter or less and binds to related targets with a K_{D} value greater than 10⁻⁴ moles/liter.

Specific binding to a certain target from a certain species does not exclude that the binding unit can also specifically bind to the analogous target from a different species. For example, specific binding to human TCR does not exclude that the binding unit (or a polypeptide comprising the same) can also specifically bind to TCR from cynomolgus monkeys.

When an ISVD is said to exhibit “improved cross-reactivity for binding to human and non-human primate TCR” compared to another ISVD, it means that for said ISVD the ratio of the binding activity (such as expressed in terms of K_{D} or k_{off}) for human TCR and for non-human primate TCR is higher than that same ratio calculated for the other ISVD in the same assay.

Good cross-reactivity for binding to human and non-human primate TCR allows for the assessment of toxicity of a multispecific T cell engaging polypeptide in preclinical studies conducted in non-human primates.

Specific binding of a binding unit to its designated target can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein.

The dissociation constant may be the actual or apparent dissociation constant, as will be clear to the skilled person. Methods for determining the dissociation constant will be clear to the skilled person, and for example include the techniques mentioned below. In this respect, it will also be clear that it may not be possible to measure dissociation constants of more than 10⁴ moles/liter or 10⁻³ moles/liter (e.g. of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}].

The affinity of a molecular interaction between two molecules can be measured via different techniques known per se, such as the well-known surface plasmon resonance (SPR) biosensor technique (see for example Ober et al. 2001, Intern. Immunology 13: 1551-1559). The term “surface plasmon resonance”, as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kₒₙ, K_{off} measurements and hence K_{D} (or K_{A}) values. This can for example be performed using the well-known BIAcore® system (BIAcore International AB, a GE Healthcare company, Uppsala, Sweden and Piscataway, NJ). For further descriptions, see Jonsson et al. (1993, Ann. Biol. Clin. 51: 19-26), Jonsson et al. (1991, Biotechniques 11: 620-627), Johnsson et al. (1995, J. Mol. Recognit. 8: 125-131), and Johnnson et al. (1991, Anal. Biochem. 198: 268-277).

Another well-known biosensor technique to determine affinities of biomolecular interactions is bio-layer interferometry (BLI) (see for example Abdiche et al. 2008, Anal. Biochem. 377: 209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA).

Alternatively, affinities can be measured in Kinetic Exclusion Assay (KinExA) (see for example Drake et al. 2004, Anal. Biochem., 328:35-43), using the KinExA® platform (Sapidyne Instruments Inc, Boise, USA). The term “KinExA”, as used herein, refers to a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen).

The GYROLAB® immunoassay system provides a platform for automated bioanalysis and rapid sample turnaround (Fraley et al. 2013, Bioanalysis 5: 1765-74).

The technology inter alia uses ISVDs that can bind to the constant domain of a TCR, so-called T cell engaging ISVDs (also referred to herein as “TCR” or “TCE ISVD”).

Accordingly, the target molecules of the TCE ISVDs used in the technology are the constant domain of the TCR.

Binding to TCR can be achieved, for example, by binding to the TCRalpha subunit and/or the TCRbeta subunit. An example is mammalian TCR. While human TCR is preferred, the versions from other species are also amenable to the present technology, for example TCR from mice, rats, rabbits, cats, dogs, goats, sheep, horses, pigs, non-human primates, such as cynomolgus monkeys (also referred to herein as “cyno”), or camelids, such as llama or alpaca.

The sequences of the TCR-α/β constant domains of human and cyno origin are provided in Table A-2 (SEQ ID NO: 86 and 96 for the constant domain of TCR a from human and cyno origin, respectively; SEQ ID NO: 87 and 97 for the constant domain of TCR β from human and cyno origin, respectively). The origin of each of these sequences, as expressed by a UniProt or Genbank files identifier, is listed for each of the aforementioned sequences in Table A-2. In house sequencing confirmed that the amino acid sequences originally derived from rhesus origin, were identical to those from cyno origin.

In one embodiment, the TCE ISVD specifically binds to the constant domain of a human T cell receptor α (TCR-α) (SEQ ID NO: 86) and/or the constant domain of the human T cell receptor β (TCR-β) (SEQ ID NO: 87), or polymorphic variants or isoforms thereof.

In one embodiment, the TCE ISVD specifically binds to the constant domain of a non-human primate TCR. In one embodiment, the non-human primate TCR is a macaque or rhesus TCR. In one embodiment, the macaque or rhesus TCR comprises the constant domain of a TCR-α of SEQ ID NO: 88 and/or of a TCR-β of SEQ ID NO: 89, or polymorphic variants or isoforms thereof.

Isoforms are alternative protein sequences that can be generated from the same gene by a single or by the combination of biological events such as alternative promoter usage, alternative splicing, alternative initiation and ribosomal frameshifting, all as known in the art.

TABLE A-2
Amino acid sequences related to TCR (″ID″ refers to the given SEQ ID NO as used herein)

| Name | ID | Amino acid sequence |
|---|---|---|
| Human TCR alpha | 86 | PNIQNPDPAVYQLRDSKSSDKSVCLFTDFDSQTNVSQSKDSDVYI |
| constant domain | | TDKTVLDMRSMDFKSNSAVAWSNKSDFACANAFNNSIIPEDTF |
| (derived from P01848) | | FPSPESSCDVKLVEKSFETDTNLNFQNLSVIGFRILLLKVAGFNLL |
| | | MTLRLWSS |
| Human TCR beta | 87 | EDLNKVFPPEVAVFEPSEAEISHTQKATLVCLATGFFPDHVELSW |
| constant domain | | WVNGKEVHSGVSTDPQPLKEQPALNDSRYALSSRLRVSATFWQ |
| (derived from P01850) | | NPRNHFRCQVQFYGLSENDEWTQDRAKPVTQIVSAEAWGRAD |
| | | CGFTSVSYQQGVLSATILYEILLGKATLYAVLVSALVLMAMVKRK |
| | | DF |
| Rhesus TCR alpha | 88 | MLLLLVLVLEVIFTLGGTRAQSVTQLDSQVSVSEGVPVLLRCNYSS |
| (AEA41863) | | SFSPYLFWYVQYPNQGLQLLLKYTSGTTLVKGINGFEAEFKKSETS |
| | | FHLTKASAHVSDAAEYFCALARGALVFGKGTRLSVIPNIQNPDPA |
| | | VYQLRGSKSNDTSVCLFTDFDSVMNVSQSKDSDVHITDKTVLD |
| | | MRSMDFKSNGAVAWSNKSDFACTSAFKDSVIPADTFFPGTESV |
| | | CDANLVEKSFETDMNLNFQNLSVIGFRILLLKVAGFNLLMTLRL |
| | | WSS |
| Rhesus TCR beta | 89 | MGFWTLCCVSFCILVAKHTDAGVIQLPRHEVTEMGKEVTLRCEP |
| (AEA41864) | | ISGHSSLFWYRQTMMRGLEFLIYFNNKSPIDDSGMPKDRFSAT |
| | | MPDASFSTLKIQPSEPRDSAVYFCASTPGQGREKLFFGSGTQLSV |
| | | LEDLKKVFPPKVAVFEPSEAEISHTQKATLVCLATGFYPDHVELS |
| | | WWVNGKEVHSGVSTDPQPLKEQPALEDSRYCLSSRLRVSATFW |
| | | HNPRNHFRCQVQFYGLSEDDEWTEDRDKPITQKISAEVWGRA |
| | | DCGFTSVSYQQGVLSATILYEILLGKATLYAVLVSALMLMAMVKR |
| | | KDF |
| Human TCR-zipper: | 90 | MNMRPVTSSVLVLLLMLRRSNGQLLEQSPQFLSIQEGENLTVYC |
| alpha chain | | NSSSVFSSLQWYRQEPGEGPVLLVTVVTGGEVKKLKRLTFQFGD |
| | | ARKDSSLHITAAQPGDTGLYLCAGAGSQGNLIFGKGTKLSVKPNI |
| | | QNPDPAVYQLRDSKSSDKSVCLFTDFDSQTNVSQSKDSDVYITD |
| | | KSVLDMRSMDFKSNSAVAWSNKSDFACANAFNNSIIPEDTFFPS |
| | | PESSCSSADLVPRGSTTAPSAQLKKKLQALKKKNAQLKWKLQAL |
| | | KKKLAQEQKLISEEDL |
| Human TCR-zipper: | 91 | MSNTVLADSAWGITLLSWVTVFLLGTSSADGGITQSPKYLFRKE |
| beta chain | | GQNVTLSCEQNLNHDAMYWYRQDPGQGLRLIYYSQIVNDFQK |
| | | GDIAEGYSVSREKKESFPLTVTSAQKNPTAFYLCASSSRSSYEQYF |
| | | GPGTRLTVTEDLKNVFPPEVAVFEPSEAEISHTQKATLVCLATGFY |
| | | PDHVELSWWVNGKEVHSGVSTDPQPLKEQPALNDSRYSLSSRL |
| | | RVSATFWQNPRNHFRCQVQFYGLSENDEWTQDRAKPVTQIVS |
| | | AEAWGRADCSSADLVPRGSTTAPSAQLEKELQALEKENAQLEW |
| | | ELQALEKELAQTGHHHHHHHHHH |
| Cyno TCR-zipper: alpha | 92 | MLLITSVLVLWMQLSQVNGQQIMQIPQYQHVQEGEDFTTYCN |
| chain (derived from | | SSTTLSNIQWYKQRPGGHPVFLIMLVKSGEVKKQKRLIFQFGEAK |
| AEA41865) | | KNSSLHITATQTTDVGTYFCATTGVNNLFFGTGTRLTVLPYIQNP |
| | | DPAVYQLRGSKSNDTSVCLFTDFDSVMNVSQSKDSDVHITDKTV |
| | | LDMRSMDFKSNGAVAWSNKSDFACTSAFKDSVIPADTFFPSPE |
| | | SSCSSADLVPRGSTTAPSAQLKKKLQALKKKNAQLKWKLQALKK |
| | | KLAQEQKLISEEDL |
| Cyno TCR-zipper: beta | 93 | MSIGLLCCAALSLLWAGPVNAGVTQTPKFQVLKTGQSMTLQCA |
| chain (derived from | | QDMNHDYMYWYRQDPGMGLRLIHYSVGEGSTEKGEVPDGYN |
| AEA41866 and | | VTRSNTEDFPLRLESAAPSQTSVYFCASSYWTGRSYEQYFGPGTR |
| AEA41868) | | LTVIEDLKKVFPPKVAVFEPSEAEISHTQKATLVCLATGFYPDHVE |
| | | LSWWVNGKEVHSGVSTDPQPLKEQPALEDSRYSLSSRLRVSATF |
| | | WHNPRNHFRCQVQFYGLSEDDEWTEDRDKPITQKISAEAWGR |
| | | ADCSSADLVPRGSTTAPSAQLEKELQALEKENAQLEWELQALEK |
| | | ELAQTGHHHHHHHHHH |
| Human TCR alpha | 94 | PNIQNPDPAVYQLRDSKSSDKSVCLFTDFDSQTNVSQSKDSDVYI |
| constant domain | | TDKTVLDMRSMDFKSNSAVAWSNKSDFACANAFNNSIIPEDTF |
| (derived from P01848) | | FPSPESSC |
| Human TCR beta | 95 | EDLNKVFPPEVAVFEPSEAEISHTQKATLVCLATGFFPDHVELSW |
| constant domain | | WVNGKEVHSGVSTDPQPLKEQPALNDSRYALSSRLRVSATFWQ |
| (derived from P01850) | | NPRNHFRCQVQFYGLSENDEWTQDRAKPVTQIVSAEAWGRAD |
| | | C |
| cynomolgus TCR-alpha | 96 | PYIQNPDPAVYQLRGSKSNDTSVCLFTDFDSVMNVSQSKDSDV |
| constant domain | | HITDKTVLDMRSMDFKSNGAVAWSNKSDFACTSAFKDSVIPAD |
| (derived from | | TFFPSPESSC |
| AEA41865) | | |
| cynomolgus TCR-beta | 97 | EDLKKVFPPKVAVFEPSEAEISHTQKATLVCLATGFYPDHVELSW |
| constant domain | | WVNGKEVHSGVSTDPQPLKEQPALEDSRYSLSSRLRVSATFWH |
| (derived from | | NPRNHFRCQVQFYGLSEDDEWTEDRDKPITQKISAEAWGRADC |
| AEA41868) | | |

Examples of TCE ISVDS that may be used in the polypeptide according to the present technology are those found in WO 2016/180969 A1, WO 2022/129637 A1, and not yet published application PCT/EP2023/065925.

In an embodiment, the TCE ISVD has a CDR1 with amino acid sequence X₁X₂VHKINFX₃G (SEQ ID NO: 2), a CDR2 with amino acid sequence HISIGDQTD (SEQ ID NO: 3) and a CDR3 with amino acid sequence X₄SRIX₅PYDY (SEQ ID NO: 4), according to Abm, wherein
a. X₁ is selected from G, W, E or D;
b. X₂ is selected from D, Y G or W;
c. X₃ is selected from L or Y;
d. X₄ is selected from F or L; and
e. X₅ is selected from W, Y, T, S or Q.

In another embodiment the TCE ISVD is selected from SEQ ID NOs: 1 and 7-21, preferably the TCE ISVD comprises SEQ ID NO: 1 or SEQ ID NO: 7.

The polypeptide according to the present technology further comprises an albumin-binding ISVD (also referred to herein as “ALB building block”, “ALB ISVD” or “ALB”). The albumin-binding ISVD serves as the masking moiety for the polypeptide according to the present technology.

In an embodiment, the albumin-binding ISVD has a CDR1 with amino acid sequence GFX₁X₂X₃X₄FGMS (SEQ ID NO: 26), a CDR2 with amino acid sequence SISGSGX₅X₆TL (SEQ ID NO: 27), and a CDR3 with amino acid sequence GGSLX₇X₈ (SEQ ID NO: 28), according to Abm, wherein
a. X₁ is T, R, K, S or P;
b. X₂ is F or Y;
c. X₃ is K, R or S;
d. X₄ is S, K, R or A
e. X₅ is S, R, T or A;
f. X₆ is D, H, V or T;
g. X₇ is S, R, T or K; and
h. X₈ is R, K, V, P or N.

In an embodiment, the albumin-binding ISVD has a CDR1 with amino acid sequence GFX₁X₂X₃X₄FGMS (SEQ ID NO: 26), a CDR2 with amino acid sequence SISGSGX₅X₆TL (SEQ ID NO: 27), and a CDR3 with amino acid sequence GGSLX₇X₈ (SEQ ID NO: 28), according to Abm, wherein
a. X₁ is T;
b. X₂ is F;
c. X₃ is R or S;
d. X₄ is S;
e. X₅ is S;
f. X₆ is D;
g. X₇ is S; and
h. X₈ is R.

In another embodiment, the albumin-binding ISVD according to the present technology has
a. a CDR1 (according to Abm) with amino acid sequence GFTFRSFGMS (SEQ ID NO: 29), or an amino acid sequence with 4, 3, 2 or 1 amino acid differences with the sequence GFTFRSFGMS;
b. a CDR2 (according to Abm) with amino acid sequence SISGSGSDTL (SEQ ID NO: 30), or an amino acid sequence with 4, 3, 2 or 1 amino acid differences with the sequence SISGSGSDTL; and
c. a CDR3 (according to Abm) with amino acid sequence GGSLSR (SEQ ID NO: 31), or an amino acid sequence with 4, 3, 2 or 1 amino acid differences with the sequence GGSLSR.

In a further embodiment, the albumin-binding ISVD according to the present technology has a CDR1 (according to Abm) with amino acid sequence GFTFRSFGMS (SEQ ID NO: 29), a CDR2 (according to Abm) with amino acid sequence SISGSGSDTL (SEQ ID NO: 30), and a CDR3 (according to Abm) with amino acid sequence GGSLSR (SEQ ID NO: 31).

In an embodiment of the present technology, the albumin-binding ISVD comprises SEQ ID NO: 32 or SEQ ID NO: 33.

Additional possible albumin-binding ISVDs have been considered. For instance, WO 04/041865 describes ISVDs binding to serum albumin (and in particular human serum albumin) that can be linked to other proteins.

Albumin-binding ISVDs have also been described in WO 2017/201488 A1 and WO 2019/222294 A1. Furthermore, the international application WO 06/122787 describes a number of ISVDs against (human) serum albumin. These ISVDs include the ISVD called Alb-1 (SEQ ID NO: 52 in WO 06/122787) and humanized variants thereof, such as Alb-8 (SEQ ID NO: 62 in WO 06/122787).

Moreover, WO2012/175400 describes a further improved version of Alb-1, called Alb-23. In an embodiment, the polypeptide comprises a serum albumin binding moiety selected from Alb-1, Alb-3, Alb-4, Alb-5, Alb-6, Alb-7, Alb-8, Alb-9, Alb-10 and Alb-23, preferably Alb-8 or Alb-23 or its variants, as shown in pages 7-9 of WO2012/175400 and the albumin binders described in WO 2012/175741, WO2015/173325, WO2017/080850, WO2017/085172, WO2018/104444, WO2018/134235, WO2018/134234.

In another embodiment, the polypeptide comprises a serum albumin binding moiety selected from Table A-3.

TABLE A-3
Serum albumin binding ISVD sequences (″ID″ refers to the SEQ ID NO as used herein)

| Name | ID | Amino acid sequence |
|---|---|---|
| Alb8 | 34 | EVQLVESGGGLVQPGNSLRLSCAASGFTFSSFGMSWVRQAPGKGLEWVSSISG |
| | | SGSDTLYADSVKGRFTISRDNAKTTLYLQMNSLRPEDTAVYYCTIGGSLSRSSQGT |
| | | LVTVSS |
| Alb8-A | 35 | EVQLVESGGGLVQPGNSLRLSCAASGFTFSSFGMSWVRQAPGKGLEWVSSISG |
| | | SGSDTLYADSVKGRFTISRDNAKTTLYLQMNSLRPEDTAVYYCTIGGSLSRSSQGT |
| | | LVTVSSA |
| Alb23 | 36 | EVQLLESGGGLVQPGGSLRLSCAASGFTFRSFGMSWVRQAPGKGPEWVSSISG |
| | | SGSDTLYADSVKGRFTISRDNSKNTLYLQMNSLRPEDTAVYYCTIGGSLSRSSQG |
| | | TLVTVSS |
| Alb23-A | 37 | EVQLLESGGGLVQPGGSLRLSCAASGFTFRSFGMSWVRQAPGKGPEWVSSISG |
| | | SGSDTLYADSVKGRFTISRDNSKNTLYLQMNSLRPEDTAVYYCTIGGSLSRSSQG |
| | | TLVTVSSA |
| Alb83 | 38 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTATYYCTIGGSLSRSSQGT |
| | | LVTVSS |
| Alb83-A | 39 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTATYYCTIGGSLSRSSQGT |
| | | LVTVSSA |
| Alb132 | 40 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTATYYCTIGGSLSRSSQ |
| | | GTLVTVSS |
| Alb132-A | 41 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTATYYCTIGGSLSRSSQ |
| | | GTLVTVSSA |
| Alb73 | 42 | EVQLVESGGGLVQPGNSLRLSCAASGFTFSSFGMSWVRQAPGKGLEWVSSISG |
| | | SGSDTLYADSVKGRFTISRDNAKTTLYLQMNSLRPEDTAVYYCTIGGSLSRSSQGT |
| | | LVKVSS |
| Alb73-A | 43 | EVQLVESGGGLVQPGNSLRLSCAASGFTFSSFGMSWVRQAPGKGLEWVSSISG |
| | | SGSDTLYADSVKGRFTISRDNAKTTLYLQMNSLRPEDTAVYYCTIGGSLSRSSQGT |
| | | LVKVSSA |
| Alb82 | 44 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTALYYCTIGGSLSRSSQGT |
| | | LVTVSS |
| Alb82-A | 45 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTALYYCTIGGSLSRSSQGT |
| | | LVTVSSA |
| Alb199 | 46 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTALYYCTIGGSLSRSSQGT |
| | | LVKVSS |
| Alb199-A | 47 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTALYYCTIGGSLSRSSQGT |
| | | LVKVSSA |
| Alb23002 | 48 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTALYYCTIGGSLSRSSQ |
| | | GTLVTVSS |
| Alb223 | 49 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTALYYCTIGGSLSRSSQ |
| | | GTLVTVSSA |
| Alb216 | 50 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTALYYCTIGGSLSRSSQ |
| | | GTLVKVSS |
| Alb216-A | 51 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTALYYCTIGGSLSRSSQ |
| | | GTLVKVSSA |

When such an ISVD binding to human serum albumin is at a C-terminal position it can exhibit a C-terminal alanine (A) or glycine (G) extension (preferably A).

In addition to the first ISVD and the second ISVD the polypeptides according to the present technology further comprise a targeting moiety.

The targeting moiety may be any moiety that can be used to ensure the polypeptide binds to the targeted cells and only become active in the vicinity of said cells.

In an embodiment, said targeting moiety is an ISVD. Said ISVD may be a VHH, a humanized VHH, a (single) domain antibody, a dAb, and a camelized VH.

In a further embodiment, the targeting moiety targets a tumor associated antigen or tumor antigen. Examples of tumor associated antigens and tumor antigens are HER2, EGFR and PSMA.

The inventors used exemplary ISVDs targeting one of HER2, EGFR and PSMA to show that the polypeptide according to the present technology can be directed to different targets. Consequently, in an embodiment the targeting moiety is selected from any one of SEQ ID NO: 22-25.

### 5.3 (In Vivo) Half-Life Extension

As was described before, the polypeptide according to the present technology comprises an ISVD that binds to human serum albumin. This albumin-binding ISVD serves as the masking moiety for the polypeptide according to the invention. Additionally, this albumin-binding ISVD provides the polypeptide according to the present technology with an extended half-life.

Besides the albumin-binding ISVD, the polypeptide according to the present technology may further comprise one or more other groups, residues, moieties or binding units, optionally linked via one or more peptidic linkers, in which said one or more other groups, residues, moieties or binding units provide the polypeptide with increased (in vivo) half-life, compared to the corresponding polypeptide without said one or more other groups, residues, moieties or binding units. In vivo half-life extension means, for example, that the polypeptide exhibits an increased half-life in a mammal, such as a human subject, after administration. Half-life can be expressed for example as t1/2beta.

The type of groups, residues, moieties or binding units is not generally restricted and may for example be selected from the group consisting of a polyethylene glycol molecule, serum proteins or fragments thereof, binding units that can bind to serum proteins, an Fc portion, and small proteins or peptides that can bind to serum proteins.

More specifically, said one or more other groups, residues, moieties or binding units that provide the polypeptide with increased half-life can be selected from the group consisting of binding units that can bind to serum albumin, such as human serum albumin, or a serum immunoglobulin, such as IgG.

### 5.4 Nucleic Acid Molecules

Also provided is a nucleic acid molecule encoding the polypeptides as disclosed herein.

A “nucleic acid molecule” (used interchangeably with “nucleic acid”) is a chain of nucleotide monomers linked to each other via a phosphate backbone to form a nucleotide sequence. A nucleic acid may be used to transform/transfect a host cell or host organism, e.g. for expression and/or production of a polypeptide. Suitable hosts or host cells for production purposes will be clear to the skilled person, and may for example be any suitable fungal, prokaryotic or eukaryotic cell or cell line or any suitable fungal, prokaryotic or eukaryotic organism. A (non-human) host or host cell comprising a nucleic acid encoding the polypeptide is also encompassed by the technology.

A nucleic acid may be for example DNA, RNA, or a hybrid thereof, and may also comprise (e.g. chemically) modified nucleotides, like PNA. It can be single- or double-stranded and is preferably in the form of double-stranded DNA. For example, the nucleotide sequences may be genomic DNA or cDNA.

The nucleic acids can be prepared or obtained in a manner known per se, and/or can be isolated from a suitable natural source. Nucleotide sequences encoding naturally occurring (poly) peptides can for example be subjected to site-directed mutagenesis, so as to provide a nucleic acid molecule encoding polypeptide with sequence variation. Also, as will be clear to the skilled person, to prepare a nucleic acid, also several nucleotide sequences, such as at least one nucleotide sequence encoding a targeting moiety and for example nucleic acids encoding one or more linkers can be linked together in a suitable manner.

Techniques for generating nucleic acids will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers.

### 5.5 Vectors

Also provided is a vector comprising the nucleic acid molecule encoding the polypeptides as disclosed herein.

A “vector” as used herein is a vehicle suitable for carrying genetic material into a cell. A vector includes naked nucleic acids, such as plasmids or mRNAs, or nucleic acids embedded into a bigger structure, such as liposomes or viral vectors.

Vectors generally comprise at least one nucleic acid that is optionally linked to one or more regulatory elements, such as for example one or more suitable promoter(s), enhancer(s), terminator(s), etc.). The vector preferably is an expression vector, i.e. a vector suitable for expressing an encoded polypeptide or construct under suitable conditions, e.g. when the vector is introduced into a (e.g. human) cell. For DNA-based vectors, this usually includes the presence of elements for transcription (e.g., a promoter and a polyA signal) and translation (e.g. Kozak sequence).

Preferably, in the vector, said at least one nucleic acid and said regulatory elements are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

Preferably, any regulatory elements of the vector are such that they are capable of providing their intended biological function in the intended host cell or host organism.

For instance, a promoter, enhancer or terminator should be “operable” in the intended host cell or host organism, by which is meant that for example said promoter should be capable of initiating or otherwise controlling/regulating the transcription and/or the expression of a nucleotide sequence—e.g., a coding sequence—to which it is operably linked.

### 5.6 Compositions

The technology also provides a composition comprising at least one polypeptide as disclosed herein, at least one nucleic acid molecule encoding a polypeptide as disclosed herein or at least one vector comprising such a nucleic acid molecule. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

### 5.7 Host Organisms

The technology also pertains to host cells or host organisms comprising the ISVDs or polypeptides as disclosed herein, the nucleic acid encoding the ISVDs or polypeptides as disclosed herein, and/or the vector comprising the nucleic acid molecule encoding the ISVDs or polypeptides as disclosed herein.

Suitable host cells or host organisms are clear to the skilled person, and are for example any suitable fungal, prokaryotic or eukaryotic cell or cell line or any suitable fungal, prokaryotic or eukaryotic organism. Specific examples include HEK293 cells, CHO cells, Escherichia coli or Pichia pastoris. The most preferred host is Pichia pastoris.

Thus, in an embodiment, the invention relates to a non-human host cell or organism expressing the polypeptide, nucleic acid or vector according to the invention.

### 5.8 Methods and Uses of the Polypeptides

The technology also provides a method for producing the polypeptides as disclosed herein. The method may comprise transforming/transfecting a host cell or host organism with a nucleic acid encoding the polypeptide, expressing the polypeptide in the host, optionally followed by one or more isolation and/or purification steps. Specifically, the method may comprise:
a) expressing, in a suitable host cell or (non-human) host organism or in another suitable expression system, a nucleic acid sequence encoding the polypeptide according to the present technology; optionally followed by:
b) isolating and/or purifying the polypeptide.

Suitable host cells or host organisms for production purposes will be clear to the skilled person, and may for example be any suitable fungal, prokaryotic or eukaryotic cell or cell line or any suitable fungal, prokaryotic or eukaryotic organism. Specific examples include HEK293 cells, CHO cells, Escherichia coli or Pichia pastoris. The most preferred host is Pichia pastoris.

The present technology further provides a method for producing an activated polypeptide, wherein the method comprises:
(a) Providing the polypeptide of the present technology, e.g., as defined above or in embodiments 1-15; and
(b) Cleaving the linker comprised in the polypeptide provided in (a) with a protease, thereby obtaining the activated polypeptide.

The present technology thus provides an activated polypeptide obtained by the above method. The activated polypeptide of the present technology comprises the second ISVD specifically binding to the constant domain of a human and non-human primate T cell receptor (TCR) present on a T cell as described herein and a targeting moiety that specifically binds a target antigen on a target cell, wherein said target antigen is not TCR or serum albumin, and wherein said target cell is not a T cell, as described herein.

The activated polypeptide of the present technology (i.e., once the linker has been cleaved by the protease):
induces T cell activation (as measured by flow cytometry) which increases by at least 20-fold, preferably at least 40-fold, more preferably at least 70-fold, most preferably at least 80-fold after cleavage of the linker by the protease compared to the polypeptide wherein the linker has not been cleaved by the protease;
induces T cell activation (as measured by flow cytometry);
induces T cell activation with an EC50 value of at most about 10⁻⁹ M, at most about 5.10⁻¹⁰ M, preferably at most about 10⁻¹⁰ M, as determined by the CD69 expression on primary T cells measured in flow cytometry;
induces T cell activation with an EC50 value of at most about 10⁻¹⁰ M, preferably at most about 10⁻¹¹ M, as determined by the CD69 expression on PBMCs measured in flow cytometry;
induces T cell activation (as determined by the CD69 expression on PBMCs measured in flow cytometry) that increases by at least 40-fold, by at least 100 fold, preferably by at least 1000-fold after cleavage of the linker by a protease compared to the polypeptide wherein the linker has not been cleaved by the protease;
has an affinity (K_{D}) for binding TCR (as measured by surface plasmon resonance) of at most about 10⁻⁷ M, preferably of at most about 5.10⁻⁸ M;
has an affinity (K_{D}) for TCR (as measured by surface plasmon resonance) that increases by at least 4-fold after cleavage of the linker by the protease compared to the polypeptide wherein the linker has not been cleaved by the protease;
induces T cell mediated cytotoxicity (as measured by an impedance-based cytotoxicity assay) that increases by at least 40-fold, preferably at least 100-fold, more preferably at least 300-fold, most preferably at least 1000-fold after cleavage of the linker by the protease compared to the polypeptide wherein the linker has not been cleaved by the protease;
induces T cell mediated cytotoxicity (as measured by an impedance-based cytotoxicity assay) with an IC50 value of at most about 5.10⁻⁹ M, at most about 10⁻⁹ M, preferably at most about 10⁻¹⁰ M, more preferably at most about 10⁻¹¹ M, most preferably at most about 5.10⁻¹² M;
induces PBMC mediated cell toxicity (as measured by an impedance-based cytotoxicity assay) that increases by at least 40-fold, preferably at least 100-fold, more preferably at least 300-fold, most preferably at least 1000-fold after cleavage of the linker by the protease compared to the polypeptide wherein the linker has not been cleaved by the protease;
induces PBMC mediated cell toxicity (as measured by an impedance-based cytotoxicity assay) with an IC50 value of at most about 10⁻⁸ M, at most about 10⁻⁹ M, preferably at most about 10⁻¹⁰ M, more preferably at most about 10⁻¹¹ M, most preferably at most about 10⁻¹² M;
induces cytokine secretion (as measured by a bead-based multiplex assay);
induces secretion of IL-6 (as measured in a bead-based multiplex assay), with an EC50 value of at most about 10⁻⁸ M, at most about 10⁻⁹ M, more preferably at most about 10⁻¹⁰ M, most preferably at most about 10⁻¹¹ M;
induces secretion of IL-6 that increases by at least 10-fold, preferably at least 100-fold after cleavage of the linker by the protease compared to the polypeptide wherein the linker has not been cleaved by the protease;
induces secretion of IFN-γ with an EC50 value of at most about 10⁻⁹ M, at most about 10⁻⁹ M, more preferably at most about 10⁻¹⁰ M, most preferably at most about 5.10⁻¹¹ M;
induces secretion of IFN-γ that increases by at least 10-fold, preferably at least 50-fold, more preferably at least 100-fold after cleavage of the linker by the protease compared to the polypeptide wherein the linker has not been cleaved by the protease;
induces secretion of TNF-α with an EC50 value of at most about 5.10⁻⁹ M, at most about 10⁻⁹ M, more preferably at most about 10⁻¹⁰ M, most preferably at most about 5.10⁻¹¹ M;
induces secretion of TNF-α that increases by at least 10-fold, preferably at least 50-fold, more preferably at least 100-fold after cleavage of the linker by the protease compared to the polypeptide wherein the linker has not been cleaved by the protease;
induces secretion of IL-2 with an EC50 value of at most about 10⁻⁸ M, at most about 10⁻⁹ M, more preferably at most about 10⁻¹⁰ M, most preferably at most about 5.10⁻¹¹ M; and/or
induces secretion of IL-2 that increases by at least 3-fold, at least 10-fold, preferably at least 100-fold after cleavage of the linker by the protease compared to the polypeptide wherein the linker has not been cleaved by the protease.

The polypeptide, nucleic acid molecule or vector as described, or the composition comprising the polypeptide, nucleic acid molecule or vector-preferably the polypeptide or a composition comprising the same- are useful as a medicament.

Accordingly, the technology provides the polypeptide, nucleic acid molecule or vector as described, or a composition comprising the polypeptide, nucleic acid molecule or vector for use as a medicament.

Also provided is the polypeptide, nucleic acid molecule or vector as described herein, or a composition comprising the polypeptide, nucleic acid molecule or vector for use in the prevention, treatment or amelioration of a disease selected from the group consisting of a proliferative disease, an inflammatory disease, an infectious disease and an autoimmune disease.

In some embodiments, provided is the polypeptide, nucleic acid molecule or vector as described herein, or a composition comprising the polypeptide, nucleic acid molecule or vector for use in the prevention, treatment or amelioration of a proliferative disease.

Additionally, provided is the polypeptide, nucleic acid molecule or vector as described herein, or a composition comprising the polypeptide, nucleic acid molecule or vector for use in the treatment of cancer.

Also provided is a method for the prevention, treatment or amelioration of a disease, wherein said method comprises administering, to a subject in need thereof, a pharmaceutically active amount of the polypeptide, nucleic acid molecule or vector as described herein, or a composition comprising the polypeptide, nucleic acid molecule or vector.

Further provided is a method for the prevention, treatment or amelioration of a disease selected from the group consisting of a proliferative disease, an inflammatory disease, an infectious disease and an autoimmune disease, wherein said method comprises administering, to a subject in need thereof, a pharmaceutically active amount of the polypeptide, nucleic acid molecule or vector as described herein, or a composition comprising the polypeptide, nucleic acid molecule or vector.

Additionally, provided is a method of treating cancer, wherein said method comprises administering, to a subject in need thereof, a pharmaceutically active amount of the polypeptide, nucleic acid molecule or vector as described herein, or a composition comprising the polypeptide, nucleic acid molecule or vector.

Further provided is the use of the polypeptide, nucleic acid molecule or vector as described herein, or a composition comprising the polypeptide, nucleic acid molecule or vector in the preparation of a medicament.

Also provided is the use of the polypeptide, nucleic acid molecule or vector as described herein, or a composition comprising the polypeptide, nucleic acid molecule or vector in the preparation of a medicament for the prevention, treatment or amelioration of a disease selected from the group consisting of a proliferative disease, an inflammatory disease, an infectious disease and an autoimmune disease.

Further provided is the use of the polypeptide, nucleic acid molecule or vector as described herein, or a composition comprising the polypeptide, nucleic acid molecule or vector in the preparation of a pharmaceutical composition, preferably for treating cancer.

A “subject” as referred to in the context of the technology can be any animal, preferably a mammal. Among mammals, a distinction can be made between humans and non-human mammals. Non-human animals may be for example companion animals (e.g. dogs, cats), livestock (e.g. bovine, equine, ovine, caprine, or porcine animals), or animals used generally for research purposes and/or for producing antibodies (e.g. mice, rats, rabbits, cats, dogs, goats, sheep, horses, pigs, non-human primates, such as cynomolgus monkeys, or camelids, such as llama or alpaca).

In the context of prophylactic and/or therapeutic purposes, the subject can be any animal, and more specifically any mammal, but preferably is a human subject.

As used herein, the terms “treat”, “treatment” and “treating” in the context of administering (a) therapy(ies) to a subject refer to the reduction or amelioration of the progression, severity, and/or duration of a disorder associated with a hyperproliferative cell disorder, e.g., cancer, and/or the amelioration of one or more symptoms thereof resulting from the administration of one or more therapies (including, but not limited to, the administration of one or more prophylactic or therapeutic agents). In specific embodiments, the terms “treat”, “treatment” and “treating” in the context of administering a therapy/therapies to a subject refer to the reduction or amelioration of the progression, severity, and/or duration of a hyperproliferative cell disorder, e.g., cancer, refers to a reduction in cancer cells by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control (e.g., a negative control such as phosphate buffered saline). In other embodiments, the terms “treat”, “treatment” and “treating” in the context of administering a therapy, or therapies, to a subject refer to the reduction or amelioration of the progression, severity, and/or duration of a hyperproliferative cell disorder, e.g., cancer, refers to no change in cancer cell number, a reduction in hospitalization time, a reduction in mortality, or an increase in survival time of the subject with cancer.

Substances (including polypeptides, nucleic acid molecules and vectors) or compositions may be administered to a subject by any suitable route of administration, for example by enteral (such as oral or rectal) or parenteral (such as epicutaneous, sublingual, buccal, nasal, intra-articular, intradermal, intramuscular, intraperitoneal, intravenous, subcutaneous, transdermal, or transmucosal) administration. Parenteral administration, such as intramuscular, subcutaneous or intradermal, administration is preferred. Most preferred is subcutaneous administration.

An effective amount of a polypeptide, a nucleic acid molecule or vector as described, or a composition comprising the polypeptide, nucleic acid molecule or vector can be administered to a subject in order to provide the intended treatment results.

One or more doses can be administered. If more than one dose is administered, the doses can be administered in suitable intervals in order to maximize the effect of the polypeptide, composition, nucleic acid molecule or vector.

Another embodiment concerns the polypeptide according to the present technology, wherein cleavage of the linker by a protease results in an activated polypeptide and, wherein the activated polypeptide induces T cell activation (as measured by flow cytometry) which increases by at least 20-fold, preferably at least 40-fold, more preferably at least 70-fold, most preferably at least 80-fold after cleavage of the protease cleavable linker compared to the polypeptide wherein the linker has not been cleaved by the protease.

“T cell activation” as used herein refers to one or more cellular response(s) of a T cell, e.g. a cytotoxic T cell, such as selected from: proliferation, differentiation, cytokine secretion, cytotoxic effector molecule release, cytotoxic activity, expression of activation markers, and redirected target cell lysis. The polypeptides of the current technology are capable of inducing T cell activation. Suitable assays to measure T cell activation are known in the art described herein, for instance as described in WO 99/54440 or by Schlereth et al. 2005 (Cancer Immunol. Immunother. 20: 1-12), or as exemplified in the Examples.

T cell activation by the polypeptides of the present technology can be monitored by upregulation of CD69, CD25 and various cell adhesion molecules, de novo expression and/or release of cytokines (e.g., IFN-γ, TNF-α, IL-6, IL-2, IL-4 and IL-10), upregulation of granzyme and perforin expression, and/or cell proliferation, membrane blebbing, activation of procaspases 3 and/or 7, fragmentation of nuclear DNA and/or cleavage of caspase substrate poly (ADPribose) polymerase. Preferably, redirected lysis of target cells by polypeptides is independent of T cell receptor specificity, presence of MHC class I and/or β2 microglobulin, and/or of any co-stimulatory stimuli. In an embodiment, the present technology relates to a polypeptide as described herein, wherein said T cell activation is independent from MHC recognition.

In an embodiment, the present technology relates to a polypeptide as described herein, wherein said T cell activation causes one or more cellular response of said T cell, wherein said cellular response is selected from the group consisting of proliferation, differentiation, cytokine secretion, cytotoxic effector molecule release, cytotoxic activity, expression of activation markers and redirected target cell lysis.

In an embodiment, the polypeptide induces T cell activation (as measured by flow cytometry) only after cleavage of the protease cleavable linker by a protease.

In another embodiment, the activated polypeptide induces T cell activation with an IC50 value of at most about 10⁻¹⁰ M, preferably at most about 10⁻¹¹ M.

By showing that T cell activation increases after the protease cleavable linker has been cleaved, the inventors have shown, as is further illustrated in the Example section, that the polypeptide according to the present technology is activatable and that its ability to recruit T cells to the target site is, at least partially, restored when the polypeptide is at said target site.

Accordingly, the present technology provides the polypeptide, nucleic acid molecule or vector as described herein, or a composition comprising the polypeptide, nucleic acid molecule or vector for use in the activation of T cells.

Also provided is a method for the activation of T cells in a subject, wherein said method comprises administering, to a subject in need thereof, a pharmaceutically active amount of the polypeptide, nucleic acid molecule or vector as described herein, or a composition comprising the polypeptide, nucleic acid molecule or vector.

Also provided is the use of the polypeptide, nucleic acid molecule or vector as described herein, or a composition comprising the polypeptide, nucleic acid molecule or vector in the preparation of a medicament for the activation of T cells.

In a further embodiment, cleavage of the linker by a protease results in an activated polypeptide, wherein the polypeptide has an affinity for TCR (as measured by surface plasmon resonance) that increases by at least 4-fold after cleavage of the protease cleavable linker compared to the polypeptide wherein the linker has not been cleaved by the protease.

By showing that the affinity for TCR increases after cleavage of the protease cleavable linker, the inventors have provided evidence, which is further illustrated by the Examples here below, that the polypeptide according to the present technology will be able to recruit more T cells to the target site once the polypeptide is activated. With a lower affinity for TCR when the polypeptide is masked, there is a lower chance of off-target activity, which in turn may result in less undesirable side effects for the patient administered the polypeptide.

In a further embodiment, cleavage of the linker by a protease results in an activated polypeptide, wherein the activated polypeptide induces T cell mediated cytotoxicity (as measured by an impedance-based cytotoxicity assay) that increases by at least 40-fold, preferably at least 100-fold, more preferably at least 300-fold, most preferably at least 1000-fold after cleavage of the protease cleavable linker compared to the polypeptide wherein the linker has not been cleaved by the protease.

The polypeptides of the present technology show redirected lysis in vitro with previously unstimulated peripheral polyclonal CD8⁺- and CD4⁺-positive T cells. The redirected lysis of target cells via the recruitment of T cells by the polypeptides of the present technology involves cytolytic synapse formation and delivery of perforin and granzymes. Cell lysis by T cells has been described, e.g. by Atkinson and Bleackley 1995 (Crit. Rev. Immunol 15(3-4): 359-384). Preferably, the engaged T cells are capable of serial target cell lysis and are not affected by immune escape mechanisms interfering with peptide antigen processing and presentation, or clonal T cell differentiation (see, for example, WO 2007/042261). In vitro, redirected lysis is seen at low picomolar concentrations, suggesting that very low numbers of the polypeptides of the present technology need to be bound to target cells for triggering T cells. Accordingly, the present technology relates to potent polypeptides. Preferably, the polypeptide of the current technology mediates killing of target cells, e.g. cancer cells, such as stimulating T cells in pore formation and delivering pro-apoptotic components of cytotoxic T cell granules.

T cell mediated cytotoxicity can be measured via an impedance-based cytotoxicity assay as set out in Examples 4 and 5 below.

In an embodiment, the activated polypeptide induces T cell mediated cytotoxicity with an IC50 value of at most about 10⁹ M, preferably at most 10¹⁰ M, more preferably at most about 10¹¹ M, most preferably at most 10¹² M.

T cell mediated cytotoxicity is a good indication of whether the T cells recruited to the target site are active. By showing that there is in an increase in T cell mediated cytotoxicity once the polypeptide has been activated through cleavage of the protease cleavable linker, the inventors have provided evidence that the activated polypeptides according to the present technology are capable of T cell mediated tumor cell killing and exerting a therapeutic effect at the target site.

Accordingly, the present technology provides the polypeptide, nucleic acid molecule or vector as described herein, or a composition comprising the polypeptide, nucleic acid molecule or vector for use in T cell mediated cytotoxicity.

Also provided is a method for T cell mediated cytotoxicity in a subject, wherein said method comprises administering, to a subject in need thereof, a pharmaceutically active amount of the polypeptide, nucleic acid molecule or vector as described herein, or a composition comprising the polypeptide, nucleic acid molecule or vector.

Also provided is the use of the polypeptide, nucleic acid molecule or vector as described herein, or a composition comprising the polypeptide, nucleic acid molecule or vector in the preparation of a medicament for T cell mediated cytotoxicity.

Another embodiment concerns the polypeptide, wherein cleavage of the linker by a protease results in an activated polypeptide and, wherein the activated polypeptide induces PBMC mediated cell toxicity (as measured by an impedance-based cytotoxicity assay) that increases by at least 40-fold, preferably at least 100-fold, more preferably at least 300-fold, most preferably at least 1000-fold after cleavage of the protease cleavable linker compared to the polypeptide wherein the linker has not been cleaved by the protease.

PBMC mediated cell toxicity is an indication of whether the immune system is engaged where the polypeptide according to the present technology is present. The inventors have shown, as is further illustrated by the Examples below, that the polypeptide is more potent and is specifically targeted once the protease cleavable linker has been cleaved off.

Accordingly, the present technology provides the polypeptide, nucleic acid molecule or vector as described herein, or a composition comprising the polypeptide, nucleic acid molecule or vector for use in PBMC mediated cytotoxicity.

Also provided is a method for PBMC mediated cytotoxicity in a subject, wherein said method comprises administering, to a subject in need thereof, a pharmaceutically active amount of the polypeptide, nucleic acid molecule or vector as described herein, or a composition comprising the polypeptide, nucleic acid molecule or vector.

Also provided is the use of the polypeptide, nucleic acid molecule or vector as described herein, or a composition comprising the polypeptide, nucleic acid molecule or vector in the preparation of a medicament for PBMC mediated cytotoxicity.

Accordingly, the present technology provides the polypeptide, nucleic acid molecule or vector as described herein, or a composition comprising the polypeptide, nucleic acid molecule or vector for use in the tumor cell killing.

Also provided is a method for the killing of tumor cells in a subject, wherein said method comprises administering, to a subject in need thereof, a pharmaceutically active amount of the polypeptide, nucleic acid molecule or vector as described herein, or a composition comprising the polypeptide, nucleic acid molecule or vector.

Also provided is the use of the polypeptide, nucleic acid molecule or vector as described herein, or a composition comprising the polypeptide, nucleic acid molecule or vector in the preparation of a medicament for tumor cell killing.

In another embodiment the activated polypeptide induces cytokine secretion (as measured by a bead-based multiplex assay) only upon cleavage of the protease cleavable linker.

In a further embodiment, the activated polypeptide induces secretion of IL-2, IL-6, IFN-γ and/or TNF-α with an EC50 value of at most about 10⁹ M, more preferably about 10¹⁰ M, most preferably about 10¹¹ M.

Serum IL-2 levels can be determined by any method as described herein and/or known in the art. Preferred and easy methods for determining IL-2 levels include immunoassays such as flow cytometry, inhibition assay, immunoprecipitation, immunohistochemistry, bead-based multiplex assay, and ELISA (see for instance Human IL-2 ELISA kit, ThermoFisher Scientific, Belgium, cat #BMS221-2).

Serum IL-6 levels can be determined by any method as described herein and/or known in the art. Preferred and easy methods for determining IL-6 levels include immunoassays such as flow cytometry, inhibition assay, immunoprecipitation, immunohistochemistry (Frozen), bead-based multiplex assay, and ELISA (such as e.g. Human IL-6 Quantiglo ELISA Kit” from R&D Systems, Minneapolis, MN, cat #Q6000B).

Serum IFN-γ levels can be determined by any method as described herein and/or known in the art. Preferred and easy methods for determining IFN-γ levels include immunoassays such as flow cytometry, inhibition assay, immunoprecipitation, immunohistochemistry, and ELISA (see for instance R&D Systems Human IFN-γ Quantikine® High Sensitivity (HS) ELISA Kit cat #HSDIF0).

Serum TNF-α levels can be determined by any method as described herein and/or known in the art. Preferred and easy methods for determining TNF-α levels include immunoassays such as flow cytometry, inhibition assay, immunoprecipitation, immunohistochemistry, and ELISA (see for instance, the Human TNF-alpha Quantikine ELISA Kit, R&D Systems, cat #DTA00D).

As illustrated below in the Examples, the method used herein to measure cytokine secretion for all above-listed cytokines was a bead-based multiplex assay using human PBMCs and adherent target cells. Cytokine binding to the beads was evaluated using a detection antibody mix (Bio-Rad, cat #1207919) followed by streptavidin-PE (Bio-Rad, cat #171304501). Between each step, the beads were washed three times with Bio-Plex wash buffer (Bio-Rad, cat #171304040) and a magnet was placed for approximately one minute to remove the wash buffer. Read-out was performed on Luminex FLEXMAP 3D. Cytokine quantification was calculated using the Bio-Plex Pro Human Cytokine Screening Panel Standards (Bio-Rad, cat #12007919).

As is commonly known, the secretion of inflammatory cytokines is a way for the immune system to deal with infection and injury to the body. However, with a systemic release of inflammatory cytokines comes the risk of so-called cytokine release syndrome (CRS). Antibody therapeutics, and other immunotherapy compounds, are known to have the potential to induce CRS in patients. CRS is characterized by high elevations of cytokines such as TNFα, IFNb, IL-Ib, IFNγ, granulocyte-macrophage colony-stimulating factor, IL-10, and IL-6. These cytokine elevations result in a plethora of clinical symptoms including fever, hypotension, organ dysfunction, respiratory failure and coagulopathy. Consequently, CRS can be fatal.

By ensuring that the polypeptide according to the present technology is only activated once it reaches its target site, the risk of CRS is reduced. Cytokines are mostly released locally at the site of disease, meaning that a systemic inflammatory response is not as likely. The inventors have shown, as further illustrated by the Example section below, that the polypeptide according to the invention indeed does not induce a strong cytokine release response until the protease cleavable linker has been cleaved off and the polypeptide becomes activated.

Thus, it has been shown that the polypeptides according to the present technology become active at the target site upon cleavage by a protease, that their potency is restored once the protease cleavable linker is cleaved off, and that they can be specifically targeted to ensure local cytokine release. This indicates that the polypeptides according to the present technology have the potential to cause less severe undesirable side effects in patients while maintaining the ability to effectively treat disease.

### FURTHER SEQUENCES

TABLE A-4
Further sequences

| ID | SEQ ID NO | Amino Acid sequence |
|---|---|---|
| TCE ISVD (688) | 1 | EVQLVESGGGVVQPGGSLRLSCVASWDVHKINFYGWYRQ |
| | | APGKEREKVAHISIGDQTDYADSAKGRFTISRDESKNTVYLQ |
| | | MNSLRPEDTALYYCRALSRIWPYDYWGQGTLVTVSS |
| TCE (E1D) ISVD (688) | 7 | DVQLVESGGGVVQPGGSLRLSCVASWDVHKINFYGWYRQ |
| | | APGKEREKVAHISIGDQTDYADSAKGRFTISRDESKNTVYLQ |
| | | MNSLRPEDTALYYCRALSRIWPYDYWGQGTLVTVSS |
| TCE ISVD (56G05) | 8 | EVQLVESGGGLVQPGGSLRLSCVASGDVHKINFLGWYRQA |
| | | PGKEREKVAHISIGDQTDYADSAKGRFTISRDESKNMVYLQ |
| | | MNSLKPEDTAVYFCRAFSRIYPYDYWGQGTLVTVSS |
| TCE (E1D) ISVD | 9 | DVQLVESGGGLVQPGGSLRLSCVASGDVHKINFLGWYRQA |
| (56G05) | | PGKEREKVAHISIGDQTDYADSAKGRFTISRDESKNMVYLQ |
| | | MNSLKPEDTAVYFCRAFSRIYPYDYWGQGTLVTVSS |
| TCE ISVD (TCE01) | 10 | EVQLVESGGGVVQPGGSLRLSCVASGYVHKINFYGWYRQA |
| | | PGKEREKVAHISIGDQTDYADSAKGRFTISRDESKNTVYLQ |
| | | MNSLRPEDTAAYYCRALSRIWPYDYWGQGTLVTVSS |
| TCE (E1D) ISVD | 11 | DVQLVESGGGVVQPGGSLRLSCVASGYVHKINFYGWYRQ |
| (TCE01) | | APGKEREKVAHISIGDQTDYADSAKGRFTISRDESKNTVYLQ |
| | | MNSLRPEDTAAYYCRALSRIWPYDYWGQGTLVTVSS |
| TCE ISVD (TCE02) | 12 | EVQLVESGGGVVQPGGSLRLSCVASGYVHKINFYGWYRQA |
| | | PGKEREKVAHISIGDQTDYANSAKGRFTISRDESKNTVYLQ |
| | | MNSLRPEDTAAYYCRALSRIWPYDYWGQGTLVTVSS |
| TCE (E1D) ISVD | 13 | DVQLVESGGGVVQPGGSLRLSCVASGYVHKINFYGWYRQ |
| (TCE02) | | APGKEREKVAHISIGDQTDYANSAKGRFTISRDESKNTVYLQ |
| | | MNSLRPEDTAAYYCRALSRIWPYDYWGQGTLVTVSS |
| TCE ISVD (TCE03a) | 14 | EVQLVESGGGVVQPGGSLRLSCVASGYVHKINFYGWYRQA |
| | | PGKEREKVAHISIGDQTDYAESAKGRFTISRDESKNTVYLQ |
| | | MNSLRPEDTAAYYCRALSRIYPYDYWGQGTLVTVSS |
| TCE (E1D) ISVD | 15 | DVQLVESGGGVVQPGGSLRLSCVASGYVHKINFYGWYRQ |
| (TCE03a) | | APGKEREKVAHISIGDQTDYAESAKGRFTISRDESKNTVYLQ |
| | | MNSLRPEDTAAYYCRALSRIYPYDYWGQGTLVTVSS |
| TCE ISVD (TCE03b) | 16 | EVQLVESGGGVVQPGGSLRLSCVASGYVHKINFYGWYRQA |
| | | PGKEREKVAHISIGDQTDYAESAKGRFTISRDESKNTVYLQ |
| | | MNSLRPEDTAAYYCRALSRIQPYDYWGQGTLVTVSS |
| TCE (E1D) ISVD | 17 | DVQLVESGGGVVQPGGSLRLSCVASGYVHKINFYGWYRQ |
| (TCE03b) | | APGKEREKVAHISIGDQTDYAESAKGRFTISRDESKNTVYLQ |
| | | MNSLRPEDTAAYYCRALSRIQPYDYWGQGTLVTVSS |
| TCE ISVD (TCE03c) | 18 | EVQLVESGGGVVQPGGSLRLSCVASGYVHKINFYGWYRQA |
| | | PGKEREKVAHISIGDQTDYAESAKGRFTISRDESKNTVYLQ |
| | | MNSLRPEDTAAYYCRALSRISPYDYWGQGTLVTVSS |
| TCE (E1D) ISVD | 19 | DVQLVESGGGVVQPGGSLRLSCVASGYVHKINFYGWYRQ |
| TCE03c) | | APGKEREKVAHISIGDQTDYAESAKGRFTISRDESKNTVYLQ |
| | | MNSLRPEDTAAYYCRALSRISPYDYWGQGTLVTVSS |
| TCE ISVD (TCE03d) | 20 | EVQLVESGGGVVQPGGSLRLSCVASGYVHKINFYGWYRQA |
| | | PGKEREKVAHISIGDQTDYAESAKGRFTISRDESKNTVYLQ |
| | | MNSLRPEDTAAYYCRALSRITPYDYWGQGTLVTVSS |
| TCE (E1D) ISVD | 21 | DVQLVESGGGVVQPGGSLRLSCVASGYVHKINFYGWYRQ |
| (TCE03d) | | APGKEREKVAHISIGDQTDYAESAKGRFTISRDESKNTVYLQ |
| | | MNSLRPEDTAAYYCRALSRITPYDYWGQGTLVTVSS |
| HER2 ISVD | 22 | EVQLVESGGGLVQAGGSLRLSCAASGITFSINTMGWYRQA |
| | | PGKQRELVALISSIGDTYYADSVKGRFTISRDNAKNTVYLQM |
| | | NSLKPEDTAVYYCKRFRTAAQGTDYWGQGTLVTVSS |
| EGFR ISVD | 23 | EVQLVESGGGLVQPGGSLRLTCAASGRTSRSYGMGWFRQ |
| | | APGKEREFVSGISWRGDSTGYADSVKGRFTISRDNAKNTVD |
| | | LQMNSLKPEDTAIYYCAAAAGSAWYGTLYEYDYWGQGTL |
| | | VTVSS |
| EGFR ISVD | 24 | EVQLVESGGGLVQAGGSLRLSCAASGRTFSSYAMGWFRQ |
| | | APGKEREFVVAINWSSGSTYYADSVKGRFTISRDNAKNTM |
| | | YLQMNSLKPEDTAVYYCAAGYQINSGNYNFKDYEYDYWG |
| | | QGTLVTVSS |
| GPC3 ISVD | 25 | EVQLVESGGGLVQPGGSLRLSCVASGFTFSSFAMTWVRRP |
| | | PGKGLEWVATITNGGVTSYRDSVKGRFTISRDNAKNTLYLE |
| | | MTSLNPEDTAVYICANARRTGPRAPTDIGSYRGQGTLVTVS |
| | | S |
| ALB ISVD | 32 | EVQLVESGGGVVQPGGSLRLSCAASGFTFRSFGMSWVRQ |
| | | APGKGPEWVSSISGSGSDTLYADSVKGRFTISRDNSKNTLYL |
| | | QMNSLRPEDTALYYCTIGGSLSRSSQGTLVTVSS |
| ALB (E1D) ISVD | 33 | DVQLVESGGGVVQPGGSLRLSCAASGFTFRSFGMSWVRQ |
| | | APGKGPEWVSSISGSGSDTLYADSVKGRFTISRDNSKNTLYL |
| | | QMNSLRPEDTALYYCTIGGSLSRSSQGTLVTVSS |
| PCL | 52 | LSGRSDNH |
| PCL | 53 | ISSGLLSGRSDNH |
| PCL | 54 | LSGRSDNHSPLGLAGS |
| PCL | 55 | DYKDDDDK |

### 6 EXAMPLES

### 6.1 Example 1: Generation of Precision Activated Polypeptides

### Cloning of ISVD Constructs

For the expression of multivalent ISVD constructs in P. pastoris, the yeast expression vectors contained the AOX1 promoter and terminator, a resistance gene for Zeocin and the coding information for the Saccharomyces cerevisiae α-mating factor signal peptide. For the expression of the multivalent ISVD constructs in CHOEBNALT85-1E9 cells or HEK Expi293F cells, the mammalian expression vectors contained the RSV-LTR promoter, a resistance gene for Zeocin and the signal peptide of a mouse light chain. The ISVD building blocks were combined with GS linkers and cloned in the expression vector via Golden Gate cloning. The expression vectors contained two Bpil restriction sites for the cloning the PCR-amplified monovalent ISVDs together with the GS linkers included in one or multiple vectors. All these elements were flanked by Bpil sites. The use of unique nucleotide overhangs for each position of the cloning cassette allows seamless ligation in a pre-defined order. After Sanger sequence confirmation, plasmid DNA derived from E. coli TOP10 was transformed by electroporation into hypercompetent P. pastoris, strain NRRL Y-11430 (ATCC 76273), after linearization, or transfected into CHOEBNALT85-1E9 or HEK Expi293F (Thermo Fisher, cat #A14527) cells.

Expression of ISVD Constructs in Pichia pastoris (Komagataella phaffii)

P. pastoris cells (NRRL Y-11430 cells; ATCC 76273) containing the ISVD construct of interest were grown for two days (at 30° C., 200 rpm) in Biotin Glycerol Culture Medium (BGCM, prepared in house). On day three, the medium was switched to Biotin Methanol Culture Medium (BMCM, prepared in house). The cell culture was further incubated at 30° C., 200 rpm for 8 hours, and expression was induced with 0.5% V methanol (100%, Biosolve, cat #136878). On the next day the constructs were induced with 0.5% V methanol (100%) in the morning, at noon and in the evening. On day five, the cells were pelleted by centrifugation (15 minutes at 1500×g) and the supernatant, containing the secreted ISVD constructs, was collected, filtered, and stored at −20° C. until purification.

### Expression of ISVD Constructs in HEK Expi293F Cells

HEK Expi293F cells were seeded at a density of 3E06 cells/mL in 30 ml of BalanCD HEK293 medium (Irvine Scientific, cat #NC1192689) supplemented with 4 mM GlutaMAX (Thermo Fisher, cat #35050061) and transfected with a DNA/PEI complex. The complex was formed by mixing 30 μg of plasmid DNA, 1.75 μg of pAX270 plasmid and 100 μg of PEI-Max transfection reagent (PolySciences, cat #24765-1) in 1.5 mL Opti-MEM I Reduced Serum Medium (Gibco, cat #31985070) and incubating for 10 minutes at room temperature. After incubation for 24 hours at 37° C., 4.5 mL BalanCD HEK293 Feed (Irvine Scientific, cat #NC1203709) was added, and the cells were incubated for another 6 days at 37° C. Cells were then harvested using the Sartoclear Dynamics® Lab V50, 0.45 μm, 1 g (Sartorius, SDLV-0050-01F0-2). All incubations were done in a humidified orbital shaker incubator at 200 rpm (throw 25 mm) in the presence of 8% CO₂.

### Expression of ISVD Constructs in CHOEBNALT85-1E9 Cells

CHOEBNALT85-1E9 cells (QMCF Technology licensed from Icosagen) were seeded at a density of 1.5E06 cells/mL in 10 ml of CHO TF medium (Xell, cat #886-0001) with 6 mM GlutaMAX™ Supplement (Gibco, cat #35050-038) and transfected with a DNA/Transfection Reagent 007 complex. The complex was formed by mixing 10 μg of plasmid DNA in 300 μL of water and 50 μg of Transfection Reagent 007 (Icosagen, cat #R007-P001) in 200 μL of water and incubating for 5 minutes at room temperature. After incubation for 1.5 hours at 37° C., 10 ml of fresh CHO TF medium with GlutaMAX™ Supplement was added, and the cells were allowed to grow for 24 hours at 37° C. Subsequently, 10 ml of fresh CHO TF medium with GlutaMAX™ Supplement and Penicillin-Streptomycin (Gibco, cat #15140-122) were added, and the cells were incubated for another 72 hours at 37° C. Then, the cell density was determined and upon reaching 3.5E06 cells/mL with a viability of >90%, 1.8 mL of Basic Feed (Xell, cat #1092-0001) was added to the cells and the incubation temperature was lowered to 30° C. Basic Feed was added again after 48 hours and once more after another 48 hours. After a final incubation of 72 hours, the medium containing the ISVD proteins was harvested by 0.22 μm filtration with diatomaceous earth (Sartoclear Dynamics Lab V50, Sartorius, cat #SDLV-0050-01E0-2) and stored at −20° C. until purification. All incubations were done in a humidified orbital shaker incubator at 200 rpm (throw 25 mm) in the presence of 8% CO₂.

### Purification of ISVD Constructs

ISVD constructs containing the anti-albumin (ALB) building block were purified on Amsphere A3 (JSR, cat #10000176-C01) resins followed by a desalting step (PD MiniTrap columns with Sephadex G25 resin, Cytiva, cat #28-9180-07) and if necessary, preparative SEC (Superdex 75 Increase 10/300 GL column, Cytiva, cat #29-1487-21) in D-PBS. N-octyl-β-d-glucopyranoside (OGP; Alpha Aesar, cat #J67390) treatment was implemented during purification/gel filtration chromatography whenever low LPS levels were required. Concentration was determined via OD280/OD340 measurement. Quality control was performed by SDS-PAGE and mass spectrometry.

The generated ISVD constructs can be found in Table 1.

TABLE 1
Overview of generated ISVD constructs

| ID | Structure | SEQ ID NO | Amino Acid sequence |
|---|---|---|---|
| T028100022 | TCR-5GS- | 115 | DVQLVESGGGVVQPGGSLRLSCVASWDVHKINFYG |
| | GPC3-A | | WYRQAPGKEREKVAHISIGDQTDYADSAKGRFTISR |
| | | | DESKNTVYLQMNSLRPEDTALYYCRALSRIWPYDYW |
| | | | GQGTLVTVSSGGGGSEVQLVESGGGLVQPGGSLRLS |
| | | | CVASGFTFSSFAMTWVRRPPGKGLEWVATITNGGV |
| | | | TSYRDSVKGRFTISRDNAKNTLYLEMTSLNPEDTAVYI |
| | | | CANARRTGPRAPTDIGSYRGQGTLVTVSSA |
| T028100011 | ALB-GS-PCL- | 116 | DVQLVESGGGVVQPGGSLRLSCAASGFTFRSFGMS |
| | TCR-5GS- | | WVRQAPGKGPEWVSSISGSGSDTLYADSVKGRFTIS |
| | GPC3-A | | RDNSKNTLYLQMNSLRPEDTALYYCTIGGSLSRSSQG |
| | | | TLVTVSSGSDYKDDDDKEVQLVESGGGVVQPGGSLR |
| | | | LSCVASWDVHKINFYGWYRQAPGKEREKVAHISIGD |
| | | | QTDYADSAKGRFTISRDESKNTVYLQMNSLRPEDTA |
| | | | LYYCRALSRIWPYDYWGQGTLVTVSSGGGGSEVQLV |
| | | | ESGGGLVQPGGSLRLSCVASGFTFSSFAMTWVRRPP |
| | | | GKGLEWVATITNGGVTSYRDSVKGRFTISRDNAKNT |
| | | | LYLEMTSLNPEDTAVYICANARRTGPRAPTDIGSYRG |
| | | | QGTLVTVSSA |
| T028100002 | ALB-GS-PCL- | 117 | DVQLVESGGGVVQPGGSLRLSCAASGFTFRSFGMS |
| | 3GS-TCR- | | WVRQAPGKGPEWVSSISGSGSDTLYADSVKGRFTIS |
| | 5GS-GPC3-A | | RDNSKNTLYLQMNSLRPEDTALYYCTIGGSLSRSSQG |
| | | | TLVTVSSGSDYKDDDDKGGSEVQLVESGGGVVQPG |
| | | | GSLRLSCVASWDVHKINFYGWYRQAPGKEREKVAHI |
| | | | SIGDQTDYADSAKGRFTISRDESKNTVYLQMNSLRPE |
| | | | DTALYYCRALSRIWPYDYWGQGTLVTVSSGGGGSEV |
| | | | QLVESGGGLVQPGGSLRLSCVASGFTFSSFAMTWVR |
| | | | RPPGKGLEWVATITNGGVTSYRDSVKGRFTISRDNA |
| | | | KNTLYLEMTSLNPEDTAVYICANARRTGPRAPTDIGS |
| | | | YRGQGTLVTVSSA |
| T028100006 | ALB-GS-PCL- | 118 | DVQLVESGGGVVQPGGSLRLSCAASGFTFRSFGMS |
| | 5GS-TCR- | | WVRQAPGKGPEWVSSISGSGSDTLYADSVKGRFTIS |
| | 5GS-GPC3-A | | RDNSKNTLYLQMNSLRPEDTALYYCTIGGSLSRSSQG |
| | | | TLVTVSSGSDYKDDDDKGGGGSEVQLVESGGGVVQ |
| | | | PGGSLRLSCVASWDVHKINFYGWYRQAPGKEREKV |
| | | | AHISIGDQTDYADSAKGRFTISRDESKNTVYLQMNSL |
| | | | RPEDTALYYCRALSRIWPYDYWGQGTLVTVSSGGGG |
| | | | SEVQLVESGGGLVQPGGSLRLSCVASGFTFSSFAMT |
| | | | WVRRPPGKGLEWVATITNGGVTSYRDSVKGRFTISR |
| | | | DNAKNTLYLEMTSLNPEDTAVYICANARRTGPRAPT |
| | | | DIGSYRGQGTLVTVSSA |
| T028100027 | TCR-5GS- | 119 | DVQLVESGGGVVQPGGSLRLSCVASWDVHKINFYG |
| | HER2-A | | WYRQAPGKEREKVAHISIGDQTDYADSAKGRFTISR |
| | | | DESKNTVYLQMNSLRPEDTALYYCRALSRIWPYDYW |
| | | | GQGTLVTVSSGGGGSEVQLVESGGGLVQAGGSLRLS |
| | | | CAASGITFSINTMGWYRQAPGKQRELVALISSIGDTY |
| | | | YADSVKGRFTISRDNAKNTVYLQMNSLKPEDTAVYY |
| | | | CKRFRTAAQGTDYWGQGTLVTVSSA |
| T028100016 | ALB-GS-PCL- | 120 | DVQLVESGGGVVQPGGSLRLSCAASGFTFRSFGMS |
| | 5GS-TCR- | | WVRQAPGKGPEWVSSISGSGSDTLYADSVKGRFTIS |
| | 5GS-HER2-A | | RDNSKNTLYLQMNSLRPEDTALYYCTIGGSLSRSSQG |
| | | | TLVTVSSGSDYKDDDDKGGGGSEVQLVESGGGVVQ |
| | | | PGGSLRLSCVASWDVHKINFYGWYRQAPGKEREKV |
| | | | AHISIGDQTDYADSAKGRFTISRDESKNTVYLQMNSL |
| | | | RPEDTALYYCRALSRIWPYDYWGQGTLVTVSSGGGG |
| | | | SEVQLVESGGGLVQAGGSLRLSCAASGITFSINTMG |
| | | | WYRQAPGKQRELVALISSIGDTYYADSVKGRFTISRD |
| | | | NAKNTVYLQMNSLKPEDTAVYYCKRFRTAAQGTDY |
| | | | WGQGTLVTVSSA |
| T028100031 | ALB-GS-PCL- | 121 | DVQLVESGGGVVQPGGSLRLSCAASGFTFRSFGMS |
| | 3GS-TCR- | | WVRQAPGKGPEWVSSISGSGSDTLYADSVKGRFTIS |
| | 5GS-HER2-A | | RDNSKNTLYLQMNSLRPEDTALYYCTIGGSLSRSSQG |
| | | | TLVTVSSGSDYKDDDDKGGSEVQLVESGGGVVQPG |
| | | | GSLRLSCVASWDVHKINFYGWYRQAPGKEREKVAHI |
| | | | SIGDQTDYADSAKGRFTISRDESKNTVYLQMNSLRPE |
| | | | DTALYYCRALSRIWPYDYWGQGTLVTVSSGGGGSEV |
| | | | QLVESGGGLVQAGGSLRLSCAASGITFSINTMGWYR |
| | | | QAPGKQRELVALISSIGDTYYADSVKGRFTISRDNAK |
| | | | NTVYLQMNSLKPEDTAVYYCKRFRTAAQGTDYWGQ |
| | | | GTLVTVSSA |
| T028200198 | 5GS-TCR- | 122 | GGGGSEVQLVESGGGVVQPGGSLRLSCVASWDVH |
| | 5GS-EGFR- | | KINFYGWYRQAPGKEREKVAHISIGDQTDYADSAKG |
| | 20GS-EGFR- | | RFTISRDESKNTVYLQMNSLRPEDTALYYCRALSRIW |
| | 35GS-ALB-A | | PYDYWGQGTLVTVSSGGGGSEVQLVESGGGLVQPG |
| | | | GSLRLTCAASGRTSRSYGMGWFRQAPGKEREFVSGI |
| | | | SWRGDSTGYADSVKGRFTISRDNAKNTVDLQMNSL |
| | | | KPEDTAIYYCAAAAGSAWYGTLYEYDYWGQGTLVT |
| | | | VSSGGGGSGGGGSGGGGSGGGGSEVQLVESGGGL |
| | | | VQAGGSLRLSCAASGRTFSSYAMGWFRQAPGKERE |
| | | | FVVAINWSSGSTYYADSVKGRFTISRDNAKNTMYLQ |
| | | | MNSLKPEDTAVYYCAAGYQINSGNYNFKDYEYDYW |
| | | | GQGTLVTVSSGGGGSGGGGSGGGGSGGGGSGGG |
| | | | GSGGGGSGGGGSEVQLVESGGGVVQPGGSLRLSCA |
| | | | ASGFTFRSFGMSWVRQAPGKGPEWVSSISGSGSDT |
| | | | LYADSVKGRFTISRDNSKNTLYLQMNSLRPEDTALYY |
| | | | CTIGGSLSRSSQGTLVTVSSA |
| T028200194 | ALB-15GS- | 123 | DVQLVESGGGVVQPGGSLRLSCAASGFTFRSFGMS |
| | TCR-5GS- | | WVRQAPGKGPEWVSSISGSGSDTLYADSVKGRFTIS |
| | EGFR-20GS- | | RDNSKNTLYLQMNSLRPEDTALYYCTIGGSLSRSSQG |
| | EGFR-A | | TLVTVSSGGGGSGGGGSGGGGSEVQLVESGGGVVQ |
| | | | PGGSLRLSCVASWDVHKINFYGWYRQAPGKEREKV |
| | | | AHISIGDQTDYADSAKGRFTISRDESKNTVYLQMNSL |
| | | | RPEDTALYYCRALSRIWPYDYWGQGTLVTVSSGGGG |
| | | | SEVQLVESGGGLVQPGGSLRLTCAASGRTSRSYGMG |
| | | | WFRQAPGKEREFVSGISWRGDSTGYADSVKGRFTIS |
| | | | RDNAKNTVDLQMNSLKPEDTAIYYCAAAAGSAWYG |
| | | | TLYEYDYWGQGTLVTVSSGGGGSGGGGSGGGGSG |
| | | | GGGSEVQLVESGGGLVQAGGSLRLSCAASGRTFSSY |
| | | | AMGWFRQAPGKEREFVVAINWSSGSTYYADSVKGR |
| | | | FTISRDNAKNTMYLQMNSLKPEDTAVYYCAAGYQIN |
| | | | SGNYNFKDYEYDYWGQGTLVTVSSA |
| T028200192 | ALB-3GS-PCL- | 124 | EVQLVESGGGVVQPGGSLRLSCAASGFTFRSFGMS |
| | 3GS-TCR- | | WVRQAPGKGPEWVSSISGSGSDTLYADSVKGRFTIS |
| | 5GS-EGFR- | | RDNSKNTLYLQMNSLRPEDTALYYCTIGGSLSRSSQG |
| | 20GS-EGFR-A | | TLVTVSSGGSLSGRSDNHGGSEVQLVESGGGVVQP |
| | | | GGSLRLSCVASWDVHKINFYGWYRQAPGKEREKVA |
| | | | HISIGDQTDYADSAKGRFTISRDESKNTVYLQMNSLR |
| | | | PEDTALYYCRALSRIWPYDYWGQGTLVTVSSGGGGS |
| | | | EVQLVESGGGLVQPGGSLRLTCAASGRTSRSYGMG |
| | | | WFRQAPGKEREFVSGISWRGDSTGYADSVKGRFTIS |
| | | | RDNAKNTVDLQMNSLKPEDTAIYYCAAAAGSAWYG |
| | | | TLYEYDYWGQGTLVTVSSGGGGSGGGGSGGGGSG |
| | | | GGGSEVQLVESGGGLVQAGGSLRLSCAASGRTFSSY |
| | | | AMGWFRQAPGKEREFVVAINWSSGSTYYADSVKGR |
| | | | FTISRDNAKNTMYLQMNSLKPEDTAVYYCAAGYQIN |
| | | | SGNYNFKDYEYDYWGQGTLVTVSSA |
| T028200179 | 5GS-TCR- | 125 | GGGGSEVQLVESGGGVVQPGGSLRLSCVASWDVH |
| | 5GS-HER2- | | KINFYGWYRQAPGKEREKVAHISIGDQTDYADSAKG |
| | 35GS-ALB-A | | RFTISRDESKNTVYLQMNSLRPEDTALYYCRALSRIW |
| | | | PYDYWGQGTLVTVSSGGGGSEVQLVESGGGLVQA |
| | | | GGSLRLSCAASGITFSINTMGWYRQAPGKQRELVALI |
| | | | SSIGDTYYADSVKGRFTISRDNAKNTVYLQMNSLKPE |
| | | | DTAVYYCKRFRTAAQGTDYWGQGTLVTVSSGGGGS |
| | | | GGGGSGGGGSGGGGSGGGGSGGGGSGGGGSEVQ |
| | | | LVESGGGVVQPGGSLRLSCAASGFTFRSFGMSWVR |
| | | | QAPGKGPEWVSSISGSGSDTLYADSVKGRFTISRDNS |
| | | | KNTLYLQMNSLRPEDTALYYCTIGGSLSRSSQGTLVT |
| | | | VSSA |
| T028200154 | ALB-15GS- | 126 | DVQLVESGGGVVQPGGSLRLSCAASGFTFRSFGMS |
| | TCR-5GS- | | WVRQAPGKGPEWVSSISGSGSDTLYADSVKGRFTIS |
| | HER2-A | | RDNSKNTLYLQMNSLRPEDTALYYCTIGGSLSRSSQG |
| | | | TLVTVSSGGGGSGGGGSGGGGSEVQLVESGGGVVQ |
| | | | PGGSLRLSCVASWDVHKINFYGWYRQAPGKEREKV |
| | | | AHISIGDQTDYADSAKGRFTISRDESKNTVYLQMNSL |
| | | | RPEDTALYYCRALSRIWPYDYWGQGTLVTVSSGGGG |
| | | | SEVQLVESGGGLVQAGGSLRLSCAASGITFSINTMG |
| | | | WYRQAPGKQRELVALISSIGDTYYADSVKGRFTISRD |
| | | | NAKNTVYLQMNSLKPEDTAVYYCKRFRTAAQGTDY |
| | | | WGQGTLVTVSSA |
| T028200057 | ALB-3GS-PCL- | 56 | EVQLVESGGGVVQPGGSLRLSCAASGFTFRSFGMS |
| | 3GS-TCR- | | WVRQAPGKGPEWVSSISGSGSDTLYADSVKGRFTIS |
| | 5GS-HER2-A | | RDNSKNTLYLQMNSLRPEDTALYYCTIGGSLSRSSQG |
| | | | TLVTVSSGGSLSGRSDNHGGSEVQLVESGGGVVQP |
| | | | GGSLRLSCVASWDVHKINFYGWYRQAPGKEREKVA |
| | | | HISIGDQTDYADSAKGRFTISRDESKNTVYLQMNSLR |
| | | | PEDTALYYCRALSRIWPYDYWGQGTLVTVSSGGGGS |
| | | | EVQLVESGGGLVQAGGSLRLSCAASGITFSINTMGW |
| | | | YRQAPGKQRELVALISSIGDTYYADSVKGRFTISRDNA |
| | | | KNTVYLQMNSLKPEDTAVYYCKRFRTAAQGTDYWG |
| | | | QGTLVTVSSA |
| T028200163 | CNB-5GS- | 57 | DVQLVESGGGVVQPGGSLRLSCAASGLTFSTNPMY |
| | EGFR-20GS- | | WYRQAPGKQRELVASISSRGITNYADSVKGRFTISRD |
| | EGFR-35GS- | | NSKNTVYLQMNSLRPEDTALYYCRLASLSSGTVYWG |
| | ALB-A | | QGTLVTVSSGGGGSEVQLVESGGGLVQPGGSLRLTC |
| | | | AASGRTSRSYGMGWFRQAPGKEREFVSGISWRGDS |
| | | | TGYADSVKGRFTISRDNAKNTVDLQMNSLKPEDTAI |
| | | | YYCAAAAGSAWYGTLYEYDYWGQGTLVTVSSGGG |
| | | | GSGGGGSGGGGSGGGGSEVQLVESGGGLVQAGGS |
| | | | LRLSCAASGRTFSSYAMGWFRQAPGKEREFVVAIN |
| | | | WSSGSTYYADSVKGRFTISRDNAKNTMYLQMNSLK |
| | | | PEDTAVYYCAAGYQINSGNYNFKDYEYDYWGQGTL |
| | | | VTVSSGGGGSGGGGSGGGGGGGGSGGGGSGGG |
| | | | GSGGGGSEVQLVESGGGVVQPGGSLRLSCAASGFTF |
| | | | RSFGMSWVRQAPGKGPEWVSSISGSGSDTLYADSV |
| | | | KGRFTISRDNSKNTLYLQMNSLRPEDTALYYCTIGGSL |
| | | | SRSSQGTLVTVSSA |
| T017000688 | TCR-FLAG3- | 58 | EVQLVESGGGVVQPGGSLRLSCVASWDVHKINFYG |
| | HIS6 | | WYRQAPGKEREKVAHISIGDQTDYADSAKGRFTISR |
| | | | DESKNTVYLQMNSLRPEDTALYYCRALSRIWPYDYW |
| | | | GQGTLVTVSSAAADYKDHDGDYKDHDIDYKDDDDK |
| | | | GAAHHHHHH |
| T028200085 | ALB-3GS-PCL- | 59 | EVQLVESGGGVVQPGGSLRLSCAASGFTFRSFGMS |
| | 3GS-TCR- | | WVRQAPGKGPEWVSSISGSGSDTLYADSVKGRFTIS |
| | 5GS-HER2-A | | RDNSKNTLYLQMNSLRPEDTALYYCTIGGSLSRSSQG |
| | | | TLVTVSSGGSLSGRSDNHSPLGLAGSGGSEVQLVESG |
| | | | GGVVQPGGSLRLSCVASWDVHKINFYGWYRQAPG |
| | | | KEREKVAHISIGDQTDYADSAKGRFTISRDESKNTVYL |
| | | | QMNSLRPEDTALYYCRALSRIWPYDYWGQGTLVTV |
| | | | SSGGGGSEVQLVESGGGLVQAGGSLRLSCAASGITFS |
| | | | INTMGWYRQAPGKQRELVALISSIGDTYYADSVKGR |
| | | | FTISRDNAKNTVYLQMNSLKPEDTAVYYCKRFRTAA |
| | | | QGTDYWGQGTLVTVSSA |
| T028201458 | ALB-3GS-PCL- | 60 | EVQLVESGGGVVQPGGSLRLSCAASGFTFRSFGMS |
| | 3GS-TCR- | | WVRQAPGKGPEWVSSISGSGSDTLYADSVKGRFTIS |
| | 5GS-HER2-A | | RDNSKNTLYLQMNSLRPEDTALYYCTIGGSLSRSSQG |
| | | | TLVTVSSGGSISSGLLSGRSDNHGGSEVQLVESGGGV |
| | | | VQPGGSLRLSCVASWDVHKINFYGWYRQAPGKERE |
| | | | KVAHISIGDQTDYADSAKGRFTISRDESKNTVYLQMN |
| | | | SLRPEDTALYYCRALSRIWPYDYWGQGTLVTVSSGG |
| | | | GGSEVQLVESGGGLVQAGGSLRLSCAASGITFSINTM |
| | | | GWYRQAPGKQRELVALISSIGDTYYADSVKGRFTISR |
| | | | DNAKNTVYLQMNSLKPEDTAVYYCKRFRTAAQGTD |
| | | | YWGQGTLVTVSSA |
| T028200056 | 5GS-TCR- | 61 | GGGGSEVQLVESGGGVVQPGGSLRLSCVASWDVH |
| | 5GS-HER2-A | | KINFYGWYRQAPGKEREKVAHISIGDQTDYADSAKG |
| | | | RFTISRDESKNTVYLQMNSLRPEDTALYYCRALSRIW |
| | | | PYDYWGQGTLVTVSSGGGGSEVQLVESGGGLVQA |
| | | | GGSLRLSCAASGITFSINTMGWYRQAPGKQRELVALI |
| | | | SSIGDTYYADSVKGRFTISRDNAKNTVYLQMNSLKPE |
| | | | DTAVYYCKRFRTAAQGTDYWGQGTLVTVSSA |
| T028200333 | 5GS-TCR- | 62 | GGGGSEVQLVESGGGVVQPGGSLRLSCVASWDVH |
| | 5GS-HER2- | | KINFYGWYRQAPGKEREKVAHISIGDQTDYADSAKG |
| | 35GS-ALB- | | RFTISRDESKNTVYLQMNSLRPEDTALYYCRALSRIW |
| | GGC | | PYDYWGQGTLVTVSSGGGGSEVQLVESGGGLVQA |
| | | | GGSLRLSCAASGITFSINTMGWYRQAPGKQRELVALI |
| | | | SSIGDTYYADSVKGRFTISRDNAKNTVYLQMNSLKPE |
| | | | DTAVYYCKRFRTAAQGTDYWGQGTLVTVSSGGGGS |
| | | | GGGGSGGGGSGGGGSGGGGSGGGGSGGGGSEVQ |
| | | | LVESGGGVVQPGGSLRLSCAASGFTFRSFGMSWVR |
| | | | QAPGKGPEWVSSISGSGSDTLYADSVKGRFTISRDNS |
| | | | KNTLYLQMNSLRPEDTALYYCTIGGSLSRSSQGTLVT |
| | | | VSSGGC |
| T028200323 | G-C-2G-ALB- | 63 | GCGGEVQLVESGGGVVQPGGSLRLSCAASGFTFRSF |
| | 3GS-PCL-3GS- | | GMSWVRQAPGKGPEWVSSISGSGSDTLYADSVKGR |
| | TCR-5GS- | | FTISRDNSKNTLYLQMNSLRPEDTALYYCTIGGSLSRS |
| | HER2-A | | SQGTLVTVSSGGSLSGRSDNHGGSEVQLVESGGGVV |
| | | | QPGGSLRLSCVASWDVHKINFYGWYRQAPGKEREK |
| | | | VAHISIGDQTDYADSAKGRFTISRDESKNTVYLQMNS |
| | | | LRPEDTALYYCRALSRIWPYDYWGQGTLVTVSSGGG |
| | | | GSEVQLVESGGGLVQAGGSLRLSCAASGITFSINTMG |
| | | | WYRQAPGKQRELVALISSIGDTYYADSVKGRFTISRD |
| | | | NAKNTVYLQMNSLKPEDTAVYYCKRFRTAAQGTDY |
| | | | WGQGTLVTVSSA |
| T028200324 | G-C-2G-ALB- | 64 | GCGGEVQLVESGGGVVQPGGSLRLSCAASGFTFRSF |
| | 15GS-TCR- | | GMSWVRQAPGKGPEWVSSISGSGSDTLYADSVKGR |
| | 5GS-HER2-A | | FTISRDNSKNTLYLQMNSLRPEDTALYYCTIGGSLSRS |
| | | | SQGTLVTVSSGGGGSGGGGSGGGGSEVQLVESGGG |
| | | | VVQPGGSLRLSCVASWDVHKINFYGWYRQAPGKER |
| | | | EKVAHISIGDQTDYADSAKGRFTISRDESKNTVYLQM |
| | | | NSLRPEDTALYYCRALSRIWPYDYWGQGTLVTVSSG |
| | | | GGGSEVQLVESGGGLVQAGGSLRLSCAASGITFSINT |
| | | | MGWYRQAPGKQRELVALISSIGDTYYADSVKGRFTIS |
| | | | RDNAKNTVYLQMNSLKPEDTAVYYCKRFRTAAQGT |
| | | | DYWGQGTLVTVSSA |
| T028200150 | CNB-5GS- | 65 | DVQLVESGGGVVQPGGSLRLSCAASGLTFSTNPMY |
| | HER2-35GS- | | WYRQAPGKQRELVASISSRGITNYADSVKGRFTISRD |
| | ALB-A | | NSKNTVYLQMNSLRPEDTALYYCRLASLSSGTVYWG |
| | | | QGTLVTVSSGGGGSEVQLVESGGGLVQAGGSLRLSC |
| | | | AASGITFSINTMGWYRQAPGKQRELVALISSIGDTYY |
| | | | ADSVKGRFTISRDNAKNTVYLQMNSLKPEDTAVYYC |
| | | | KRFRTAAQGTDYWGQGTLVTVSSGGGGSGGGGSG |
| | | | GGGSGGGGSGGGGSGGGGSGGGGSEVQLVESGG |
| | | | GVVQPGGSLRLSCAASGFTFRSFGMSWVRQAPGKG |
| | | | PEWVSSISGSGSDTLYADSVKGRFTISRDNSKNTLYL |
| | | | QMNSLRPEDTALYYCTIGGSLSRSSQGTLVTVSSA |
| T017000672 | TCR-35GS- | 66 | DVQLVESGGGVVQPGGSLRLSCVASGYVHKINFYG |
| | CNB-35GS- | | WYRQAPGKEREKVAHISIGDQTDYADSAKGRFTISR |
| | CNB-A | | DESKNTVYLQMNSLRPEDTALYYCRALSRIWPYDYW |
| | | | GQGTLVTVSSGGGGSGGGGSGGGGSGGGGSGGG |
| | | | GSGGGGSGGGGSEVQLVESGGGLVQAGDSLRLSCA |
| | | | ASGRTFSSYAMGWFRQAPGKEREFVAAISWSDGST |
| | | | YYADSVKGRFTISRDNAKNTVYLQMNSLKPEDTAVY |
| | | | YCAADLTSTNPGSYIYIWAYDYWGQGTLVTVSSGGG |
| | | | GSGGGGSGGGGSGGGGSGGGGSGGGGSGGGGSE |
| | | | VQLVESGGGLVQAGDSLRLSCAASGRTFSSYAMGW |
| | | | FRQAPGKEREFVAAISWSDGSTYYADSVKGRFTISRD |
| | | | NAKNTVYLQMNSLKPEDTAVYYCAADLTSTNPGSYI |
| | | | YIWAYDYWGQGTLVTVSSA |
| T017000698 | TCR-9GS- | 67 | DVQLVESGGGVVQPGGSLRLSCVASGYVHKINFYG |
| | ALB-A | | WYRQAPGKEREKVAHISIGDQTDYADSAKGRFTISR |
| | | | DESKNTVYLQMNSLRPEDTAAYYCRALSRIWPYDYW |
| | | | GQGTLVTVSSGGGGSGGGSEVQLVESGGGVVQPG |
| | | | GSLRLSCAASGFTFRSFGMSWVRQAPGKGPEWVSSI |
| | | | SGSGSDTLYADSVKGRFTISRDNSKNTLYLQMNSLRP |
| | | | EDTALYYCTIGGSLSRSSQGTLVTVSSA |

Notes:
PCL: Protease Cleavable Linker; CNB: CONTROL ISVD; TCR: anti-TCR ISVD; ALB: anti-Albumin ISVD; GPC3: anti-GPC3 ISVD; HER2: anti-HER2 ISVD; EGFR: anti-EGFR ISVD; GS/3GS/5GS: GlySer stretch.

### 6.2 Example 2: Identification of Precision Activated ISVD Constructs

Multiple ISVDs targeting the T cell receptor were tested for masking potential and activation after cleavage of the protease cleavable linker.

Firstly, these ISVDs targeting the T cell receptor were evaluated in cytotoxicity assays to see if their functionality can be restored in presence of a 3GS or 5GS amino acid stretch at the N-terminus (3GS or 5GS resembles the possible number of amino acids at the N-terminus of the anti-TCR ISVD after cleavage of the protease cleavable linker).

In this Example, the following ISVD constructs were tested: T028100022, T028100011, T028100002, T028100006 (GPC3 ISVD constructs) and T028100016, T028100027, T028100031 (HER2 ISVD constructs). All ISVD constructs comprise the T cell engaging ISVD T017000688 (also referred to as TCR00688) and the human serum albumin binding ISVD ALB next to a targeting ISVD, and some constructs additionally contain a protease cleavable linker (PCL).

ISVD constructs containing a PCL (T028100002, T028100006, T028100011, T028100016 and T028100031) were cleaved by enterokinase (EK), light chain (New England Biolabs, cat #P8070S) and incubated at room temperature for 16 hours in D-PBS (Life Technologies-Gibco, cat #14190-250)+2 mM CaCl₂) (Merck Chemicals, cat #1.42002). Enterokinase treatment of masked ISVD constructs with a protease cleavable linker resulted in activated Glypican-3 and HER2 ISVD constructs and are referred to as T028100002_EK, T028100006_EK, T028100011_EK, T028100016_EK and T028100031_EK, respectively, in the figures and tables in this Example.

### Impedance-Based Cytotoxicity Assay

Precision activated ISVD constructs were characterized for redirected T cell mediated killing in an impedance-based cytotoxicity assay using human primary effector T cells and adherent target cells. Changes in impedance induced by the adherence of target cells to the surface of an electrode were measured using the xCELLigence® instrument (Roche). T cells are non-adherent and therefore do not impact the impedance measurements. The xCELLigence® RTCA MP instrument quantifies the changes in electrical impedance, displaying them as a dimensionless parameter, termed “cell index”, which is directly proportional to the total area of tissue-culture well that is covered by cells.

To each well of a 96 E-plate (ACEA Biosciences; 05 232 368 001) 50 μL assay medium, consisting of RPMI medium (Life Technologies-Gibco, cat #72400-021), 10% heat-inactivated Fetal Bovine Serum (FBS, Sigma-Aldrich, cat #F9665), 1× sodium pyruvate (Life Technologies-Gibco, cat #11360-039), and 1% penicillin/streptomycin (Life Technologies-Gibco, cat #15140-122), was added. Outer wells were not used and were filled with 200 μL assay medium or D-PBS (Life Technologies-Gibco, cat #14190-094).

The 96 E-plate was placed in the xCELLigence® station (in the 37° C. incubator at 5% CO₂) and a single measurement was performed to measure background impedance of the assay medium, in absence of cells. Subsequently, 50 μL target cells (2.0E+04 cells/well) in assay medium were seeded onto the 96 E-plate, and 50 μL of serially diluted ISVD construct solutions (4× concentration) in assay medium containing 200 μM Alburex HSA (CSL Behring, 2160-679—final concentration of 50 μM), was added (Final volume in well=200 μL).

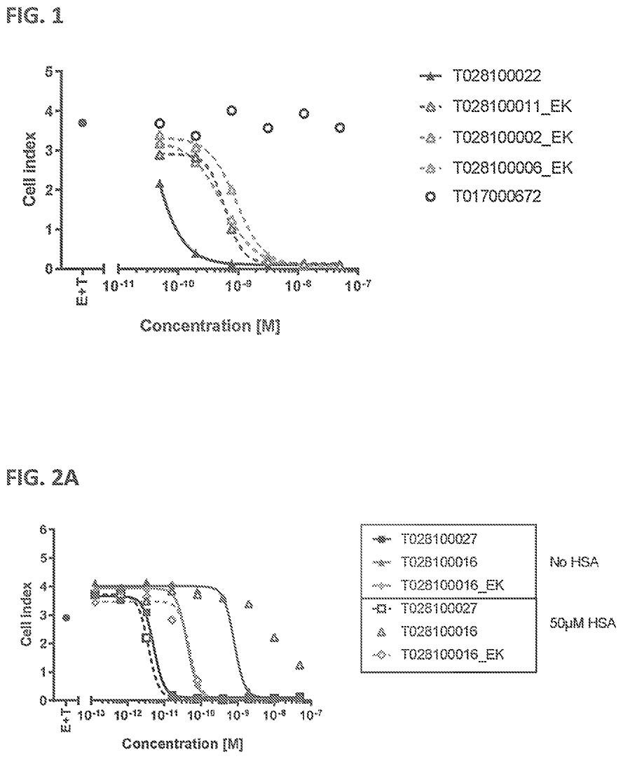

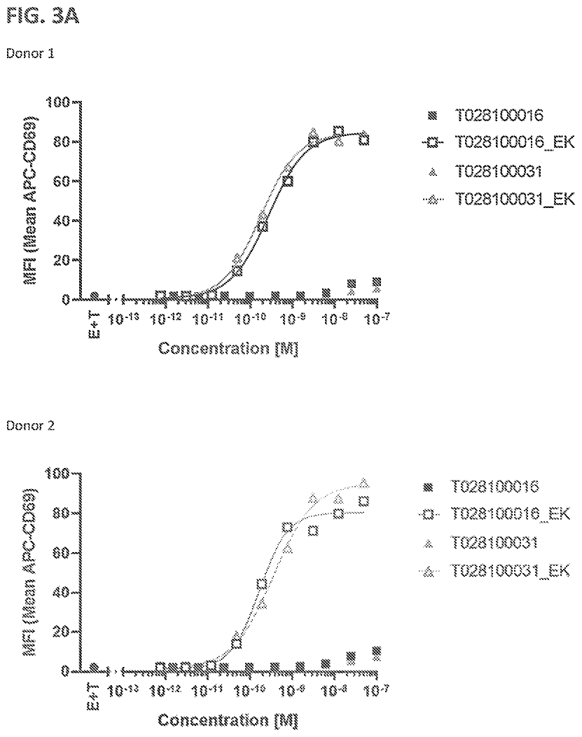

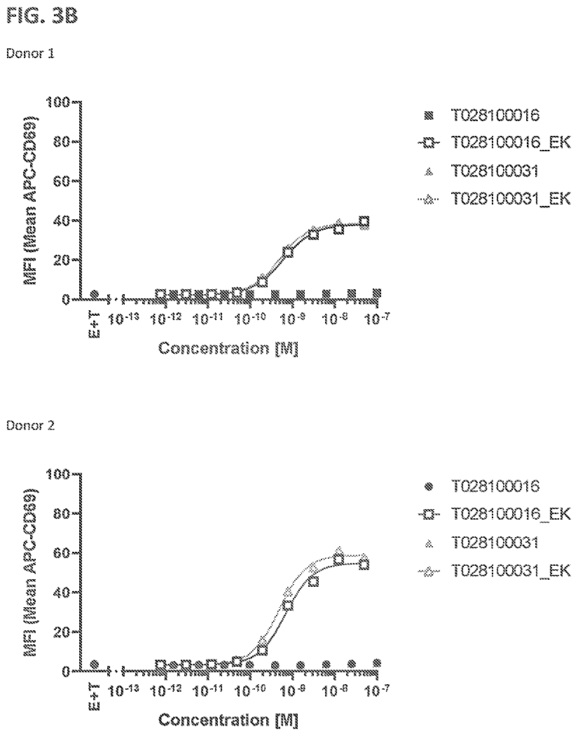

After 30 minutes at room temperature, 50 μl of primary T cells (3.0E+05 or 2.0E+05 cells/well) in assay medium were added per well to achieve an effector to target (E:T) ratio of 15:1 (FIG. 1, FIG. 2) or 10:1 (FIG. 3). The plate was placed in the xCELLigence® station and impedance was measured every 15 minutes for 4 days. The data was analyzed at a fixed time point (75 hours). Results of the measurements can be seen in FIGS. 1 and 2.

### Flow Cytometry-Based Read-Out for T Cell Activation

Precision activated ISVD constructs were characterized for T cell activation (CD69) in flow cytometry using human primary effector T cells and adherent target cells.

The target cells and primary T cells were harvested 24 hours after adding the serially diluted ISVD construct solutions and effector cells to the target cells. Target and effector cells were resuspended in FACS buffer consisting of D-PBS (Life Technologies-Gibco, cat #14190-094), 2% FBS (Sigma-Aldrich, cat #F7524), and 0.05% NaN3 (Acros Organics, cat #19038), and were transferred to a 96-well V-bottom plate. The cells were diluted in Zombie NIR™ Fixable Viability Kit (Biolegend, cat #423106) in D-PBS and incubated for 15 minutes at room temperature in the dark. The cells were stained in 100 μL antibody cocktail (CD45 (Biolegend, cat #368522), CD4 (Biolegend, cat #317420), CD8 (Biolegend, cat #344712), CD69 (Biolegend, cat #310910)) diluted in FACS buffer and incubated for 30 minutes at 4° C. in the dark. Between each step, the cells were centrifuged twice for 2 minutes at 300 g at 4° C. and washed with 100 μL/well FACS buffer. The cells were diluted in 50 μL FACS buffer and read-out was performed on MACSQuantX (Miltenyi Biotec). Results of the measurements can be seen in FIG. 3 and Table 2.

### Results

As illustrated in FIG. 1, an ISVD construct containing ISVD TCR00688 with a 3GS (T028100002 EK treated) or 5GS (T028100006 EK treated) amino acid stretch at its N-terminus, resulted in complete killing of the target cells.

Additionally, it has been shown that ISVD TCR00688 was susceptible to masking, i.e., the albumin binding ISVD at the N-terminus reduces the potency of the ISVD and this effect is stronger in case human serum albumin (HSA) is added to the assay. As can be seen from FIG. 2 and FIG. 3, cleaving off the albumin binding ISVD at least partially restores the ability of the HER2 or GPC3 ISVD construct to induce killing of tumor cells and to induce T cell activation (Table 2).

This means that the ISVD TCR00688 can successfully be masked, while its activity is restored after cleavage by a protease.

TABLE 2
T cell activation on HCC1954 (a) and HepG2 (b) cells for a masked ISVD
construct (T028100016 and T028100031) compared to an activated (enterokinase treated)
ISVD construct (T028100016_EK and T028100031_EK).
(a) CD69 readout on HCC1954 cells

| | EC50 [M] | |
|---|---|---|
| | Donor 1 | Donor 2 |
| | MFI | MFI |
| Nanobody ID | (Mean CD69-APC) | (Mean CD69-APC) |
| T028100016 | iDRC | iDRC |
| T028100016_EK | 2.71E−10 | 1.70E−10 |
| T028100031 | iDRC | iDRC |
| T028100031_EK | 1.78E−10 | 3.60E−10 |

MFI: Mean Fluorescence Intensity/APC: Allophycocyanin/iDRC: incomplete dose-response curve

(b) CD69 read-out on HepG2 cells

| | EC50 [M] | |
|---|---|---|
| | Donor 1 | Donor 2 |
| | MFI | MFI |
| Nanobody ID | (Mean CD69-APC) | (Mean CD69-APC) |
| T028100016 | No T cell activation | No T cell activation |
| T028100016_EK | 6.09E−10 | 6.89E−10 |
| T028100031 | No T cell activation | No T cell activation |
| T028100031_EK | 5.01E−10 | 4.82E−10 |

MFI: Mean Fluorescence Intensity/APC: Allophycocyanin

### 6.3 Example 3: Affinity Measurements for Active, Masked and Activated ISVD Constructs

In this Example, the following ISVD constructs were tested: T028200198, T028200194, T028200192 (EGFR ISVD constructs) and T028200179, T028200154, T028200057 (HER2 ISVD constructs). The EGFR ISVD constructs contain two different ISVDs targeting EGFR (SEQ ID NO: 23 and SEQ ID NO: 24). ISVD constructs containing a protease cleavable linker (T028200192, T028200057) were pre-treated with Human u-Plasminogen Activator (uPA)/Urokinase (R&D Systems, cat #1310-SE) in D-PBS (Life Technologies-Gibco, cat #14190-250) for 20 hours at room temperature. uPA treatment of masked ISVD constructs with a protease cleavable linker resulted in activated EGFR and HER2 ISVD constructs and are referred to as T028200192_uPA and T028200057_uPA, respectively, in the tables in this Example.

### Off-Rate Determination Towards Human TCRαβ-Zipper Protein

Binding by purified, trivalent and tetravalent TCR ISVD constructs (TCR-EGFR-EGFR-ALB, ALB-TCR-EGFR-EGFR and TCR-HER2-ALB, ALB-TCR-HER2) to human TCRαβ Zipper protein (huTCR(2XN9)-zipper, in house produced) was probed by Surface Plasmon Resonance (SPR) (Bio-rad Laboratories, Inc., ProteOn XPR36). The target was immobilized on a GLC sensor chip (short matrix, normal capacity) using standard amine coupling chemistry. In total 8 ISVD constructs, as listed above, were injected at 6 different concentrations in a multi-cycle kinetics (MCK) experiment. As this measurement was performed in the absence and presence of 30 μM HSA (Sigma-Aldrich, cat #A8763), the samples were diluted in HBS-P+ buffer (Cytiva, cat #BR100671) with or without the addition of 30 μM HSA, prior to injecting the analytes. Each concentration of the ISVD constructs was injected for 120s and dissociation was assessed for 600s.

Data was double referenced by subtracting a reference analyte lane and a blank buffer injection. Affinity constants (ka, kd and KD) were calculated applying the Langmuir 1:1 interaction model using the ProteOn Manager 3.1.0 (Bio-rad Laboratories, Inc., Version 3.1.0.6). Results of the measurements can be seen in Tables 3 and 4.

### Results

Affinity measurements for the T cell receptor (TCR) protein show a 10-fold drop in affinity of the masked compounds (T028200194 and T028200192) compared to a non-masked ISVD construct (T028200198), illustrating the HSA-mediated masking of TCR00688. Releasing the masking moiety results in a 4-fold better affinity of the uPA activated compound (T028200192_uPA) compared to the masked compounds. The difference in affinity between the non-masked ISVD construct (T028200198) and the uPA activated compound (T028200192_uPA) can be explained by the additional amino acid stretch at the N-terminus of the latter construct.

This shows that the ISVD constructs' affinity can effectively be restored upon cleavage by a protease. These results support the observations of the T cell mediated target cell killing in Example 2 as shown in FIG. 2.

Similar results were obtained for the EGFR ISVD construct (Table 3) as well as for the HER2 ISVD construct constructs (Table 4).

TABLE 3
Affinity constants of the binding by HSA-mediated masked EGFR ISVD construct to human TCRαβ
Zipper protein, as measured by Surface Plasmon Resonance (SPR)

| | | ka | kd | KD | |
|---|---|---|---|---|---|
| | ID | 1/Ms | 1/s | M | |
| Non-masked Masked Masked Activated by uPA cleavage | T028200198 T028200194 T028200192 T028200192_uPA | 1.79E+04 5.07E+03 6.41E+03 1.92E+04 | 1.74E−04 6.12E−04 6.99E−04 5.15E−04 | 9.71E−09 1.21E−07 1.09E−07 2.68E−08 | 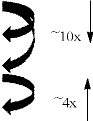 |

TABLE 4
Affinity constants of the binding by HSA-mediated masked HER2 ISVD construct to human TCRαβ
Zipper protein, as measured by Surface Plasmon Resonance (SPR)

| | | ka | kd | KD | |
|---|---|---|---|---|---|
| | ID | 1/Ms | 1/s | M | |
| Non-masked Masked Masked Activated by uPA cleavage | T028200179 T028200154 T028200057 T028200057_uPA | 1.81E+04 8.09E+03 9.11E+03 2.57E+04 | 1.80E−04 6.87E−04 7.99E−04 5.71E−04 | 9.95E−09 8.49E−08 8.78E−08 2.22E−08 | 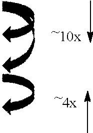 |

### 6.4 Example 4: T Cell Mediated Tumor Cell Killing of Precision Activated ISVD Constructs

In this Example, the following ISVD constructs were tested: T028200194, T028200192 (EGFR ISVD constructs); T028200154, T028200057 (HER2 ISVD constructs) and 3 PSMA ISVD constructs. ISVD constructs containing a protease cleavable linker (T028200192, T028200057) were pre-treated with Human u-Plasminogen Activator (uPA)/Urokinase (R&D Systems, cat #1310-SE) in D-PBS (Life Technologies-Gibco, cat #14190-250) for 20 hours at room temperature. uPA treatment of masked ISVD constructs with a protease cleavable linker resulted in activated EGFR and HER2 ISVD constructs and are referred to as T028200192_uPA and T028200057_uPA, respectively, in the figures and tables in this Example.

Two ISVD constructs containing Prostate Specific Antigen (PSA) cleavable linker (PSMA-TCE #2, PSMA-TCE #3) were pre-treated with PSA (R&D Systems, cat #1344-SE), which was first pre-activated (5 minutes at 37° C.) with bacterial Thermolysin (R&D Systems, cat #3097-ZN), for 20 hours at room temperature. PSA treatment of masked ISVD constructs with a protease cleavable linker resulted in activated PSMA constructs and are referred to as PSMA-TCE #2_PSA and PSMA-TCE #3_PSA in the figures and tables in this Example. As masked PSMA ISVD construct (PSMA-TCE #1), the PSA cleavable linker was replaced with a non-cleavable 15GS linker, similar to the masked EGFR and HER2 ISVD constructs.

### Impedance-Based Cytotoxicity Assay:

Precision activated ISVD constructs were characterized for redirected T cell mediated killing in an impedance-based cytotoxicity assay using human primary effector T cells and adherent target cells. Changes in impedance induced by the adherence of target cells to the surface of an electrode were measured using the xCELLigence® instrument (Roche). T cells are non-adherent and therefore do not impact the impedance measurements. The xCELLigence® RTCA MP instrument quantifies the changes in electrical impedance, displaying them as a dimensionless parameter termed cell index, which is directly proportional to the total area of tissue-culture well that is covered by cells. To each well of a 96 E-plate (ACEA Biosciences; 05 232 368 001) 50 μl assay medium, consisting of RPMI medium (Life Technologies-Gibco, cat #72400-021), 10% heat-inactivated Fetal Bovine Serum (FBS, Sigma-Aldrich, cat #F9665), 1× sodium pyruvate (Life Technologies-Gibco, cat #11360-039), 1% penicillin/streptomycin (Life Technologies-Gibco cat #15140-122)) was added. Outer wells were not used and were filled with 200 μl assay medium or D-PBS (Life Technologies-Gibco, cat #14190-094). The 96 E-plate was placed in the xCELLigence® station (in the 37° C. incubator at 5% CO₂) and a single measurement was performed to measure background impedance of the assay medium, in absence of cells. Subsequently, 50 μL target cells (density is target cell line dependent as indicated in Table 5) in assay medium were seeded onto the 96 E-plate. The plate was placed in the xCELLigence® station and impedance was measured every 15 minutes for 20-24 hours. Thereafter, 50 μL of serially diluted ISVD construct solutions (4× concentration) in assay medium containing 200 μM Alburex HSA (CSL Behring, 2160-679-final concentration of 50 μM), was added. After 30 minutes at room temperature, 50 μL of primary T cells in assay medium were added per well to achieve an effector to target (E:T) ratio of 5:1 or 10:1 (NCI-H292 cells) (Final volume=200 μL). The plate was placed in the xCELLigence® station and impedance was measured every 15 minutes for 4 days. The data was analyzed at various time points depending on the cell growth of the target cells (as indicated in Table 5) and normalized to the time point that compounds and effector cells were added.

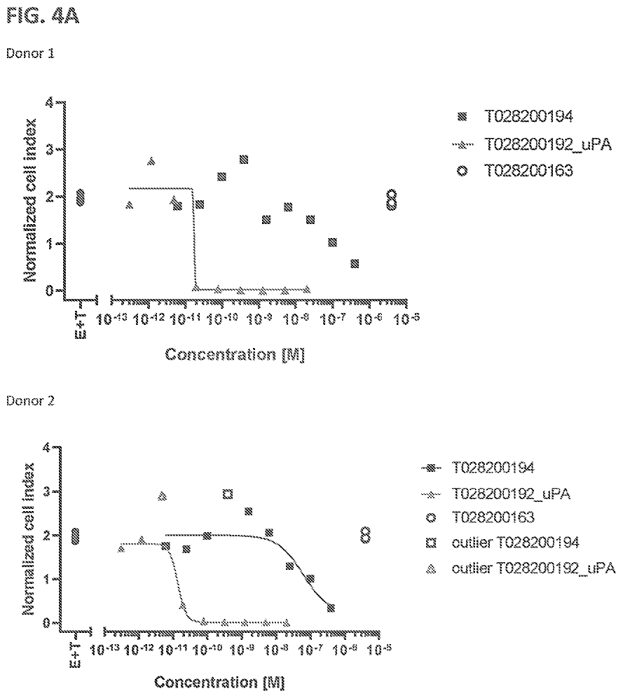

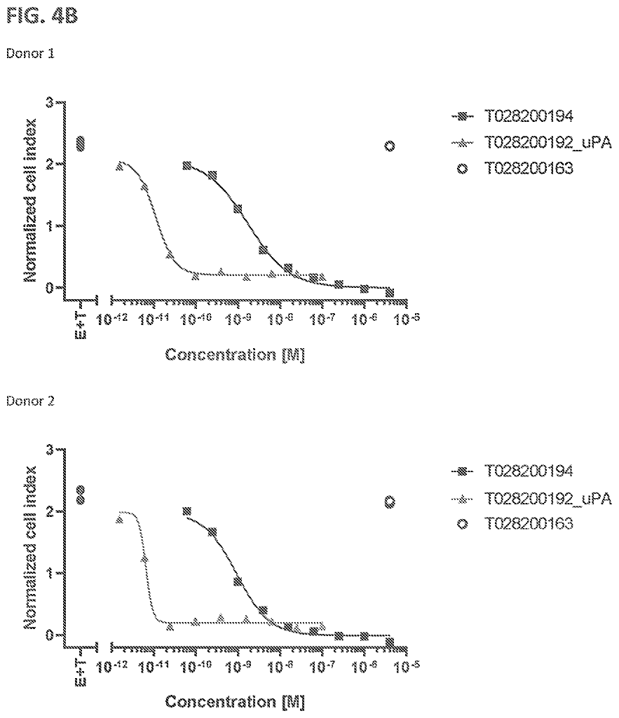

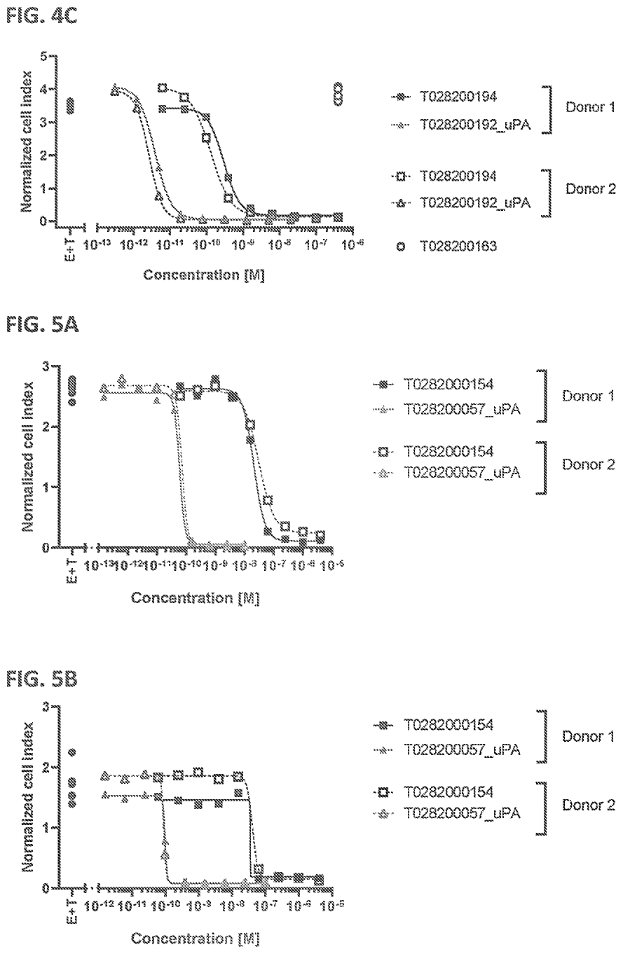

Results of the measurements are shown in FIGS. 4-6 and Tables 6-8.

TABLE 5
Overview of seeding density of target cell lines and time point
of analysis. Note that time point of analysis for LNCaP cells
deviates from the protocol due to slower growth of the cells.

| | Seeding density/ | Time points | |
|---|---|---|---|
| Cell line | 96-well | of analysis | |
| NCI-H292 | 6.5E+03 | 20-96 | h |
| LS174T | 1.5E+04 | 20-160 | h |
| LoVo | 2.0E+04 | 19-96 | h |
| HCC1954 | 1.0E+04 | 22-94 | h |
| BT-474 | 2.0E+04 | 21-93 | h |
| ZR-75-1 | 1.5E+04 | 22-120 | h |
| BT-20 | 1.0E+04 | 22-90 | h |
| BT-549 | 1.5E+04 | 20-72 | h |
| HEK293T human FOLH1 | 1.0E+04 | 24-130 | h |
| LNCaP | 2.0E+04 | 50-96 | h |
| 22RV1 | 3.0E+04 | 23-96 | h |

### Results

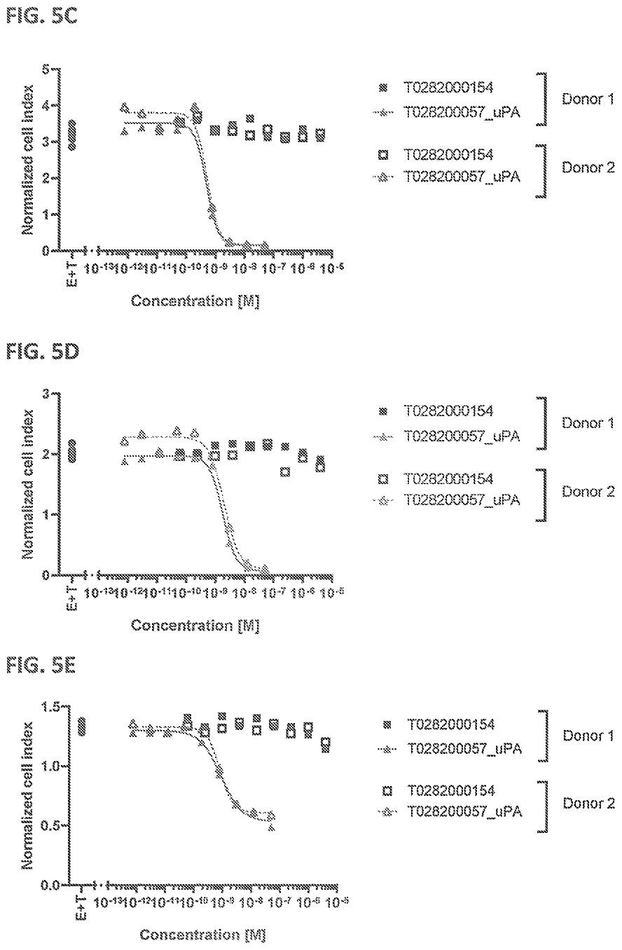

The masked EGFR ISVD construct (T028200194) shows a clear drop in potency compared to the uPA protease activated EGFR ISVD construct (T028200192_uPA). The fold masking shows to be greater on the NCI-H292 cells, followed by the LS174T cells and the LoVo cells (FIG. 4, Table 6). Similarly, the masked HER2 ISVD construct (T028200154) and the uPA protease activated HER2 ISVD construct (T028200057_uPA) were tested on multiple cell lines expressing HER2. As shown in FIG. 5, the fold difference (calculated from the IC50 values for the activated and masked ISVD construct) is at least 300-400-fold for cell lines HCC1954 and BT-474, while >1000 fold for cell lines ZR-75-1, BT-20, BT-549 (Table 7). The results are consistent across different T cell donors.

TABLE 6
Overview of the IC50 values and the fold masking for the masked EGFR
ISVD construct and the uPA protease activated EGFR ISVD construct.

| | T028200194 [IC50, M] | | T028200192_uPA [IC50, M] | | Fold masking | |
|---|---|---|---|---|---|---|
| | D1639 | D1640 | D1639 | D1640 | D1639 | D1640 |
| NCI-H292 | >1.00E−07 | 6.08E−08 | 1.74E−11 | 1.37E−11 | >1000 | 4424 |
| LS174T | 1.70E−09 | 9.28E−10 | 1.12E−11 | 6.52E−12 | 152 | 142 |
| LoVo | 2.94E−10 | 1.29E−10 | 3.87E−12 | 2.64E−12 | 76 | 49 |

TABLE 7
Overview of the IC50 values and the fold masking for the masked
HER2 ISVD construct and the uPA protease activated HER2 ISVD
construct. Data is confirmed with 2 T cell donors.

| | T028200154 [IC50, M] | | T028200057_uPA [IC50, M] | | Fold masking | |
|---|---|---|---|---|---|---|
| | D1485 | D1487 | D1485 | D1487 | D1485 | D1487 |
| HCC1954 | 2.07E−08 | 3.08E−08 | 5.95E−11 | 7.22E−11 | >300x | >400x |
| BT-474 | 3.49E−08 | 4.11E−08 | 9.74E−11 | 9.01E−11 | >300x | >400x |
| ZR-75-1 | no killing | no killing | 5.02E−10 | 5.33E−10 | >1000x | >1000x |
| BT-20 | no killing | no killing | 1.81E−09 | 2.01E−09 | >1000x | >1000x |
| BT-549 | no killing | no killing | 9.00E−10 | 8.35E−10 | >1000x | >1000x |

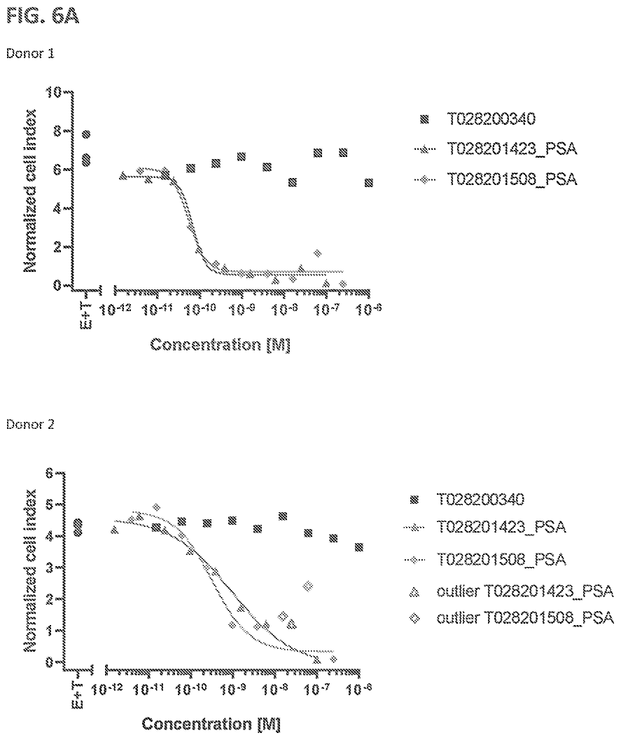

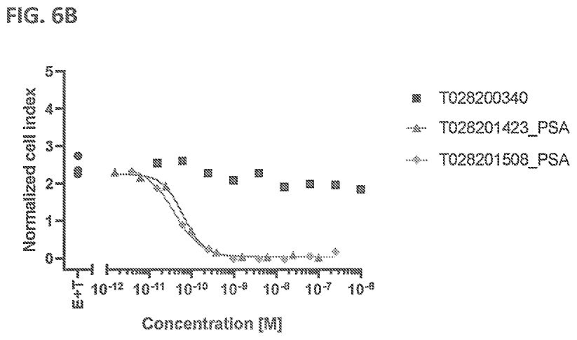

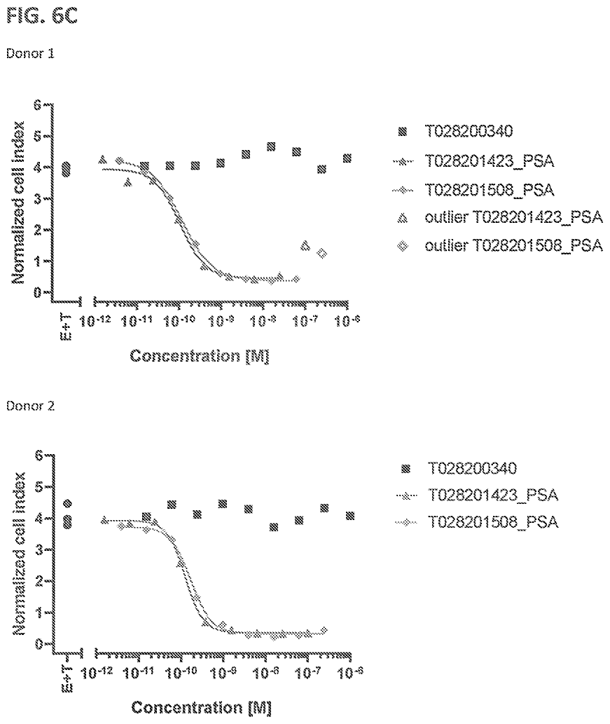

Masking of ISVD TCR00688 was also confirmed using a PSMA ISVD construct. The masked PSMA ISVD construct (PSMA-TCE #1) shows limited killing efficiency. Activation by cleavage of the PSA cleavable linker (PSMA-TCE #2_PSA, PSMA-TCE #3_PSA) results in T cell mediated killing of PSMA expressing cells (FIG. 6 and Table 8).

With these results, HSA-mediated masking of an ISVD construct containing ISVD TCR00688 is illustrated with 3 different tumor antigens. Releasing the masking moiety by proteases like uPA or PSA, resulted in efficient killing of tumor cell lines by the activated ISVD construct. The fold difference in killing potency between masked and activated ISVD construct is also driven by the expression level of the tumor antigen, especially with PSMA and HER2 ISVD construct and in lesser extent with EGFR ISVD construct.

TABLE 8
IC50 values and the fold masking for the masked PSMA ISVD construct
and the PSA protease activated PSMA ISVD construct.

| | PSMA-TCE#1 | PSMA-TCE#2 | PSMA-TCE#3 | Fold masking |
|---|---|---|---|---|
| | [IC50, M] | [IC50, M] | [IC50, M] | non-masked vs masked |
| | Donor 1 | Donor 1 | Donor 1 | Donor 1 |
| HEK293T FOLH1 | no killing | 6.85E−11 | 5.52E−11 | >1000x |
| LNCaP | no killing | 6.37E−11 | 3.88E−11 | >1000x |
| 22RV1 | no killing | 1.26E−10 | 1.72E−10 | >1000x |
| | PSMA-TCE#1 | PSMA-TCE#2 | PSMA-TCE#3 | Fold masking |
| | [IC50, M] | [IC50, M] | [IC50, M] | non-masked vs masked |
| | Donor 2 | Donor 2 | Donor 2 | Donor 2 |
| HEK293T FOLH1 | no killing | 9.72E−10 | 3.29E−10 | >1000x |
| 22RV1 | no killing | 1.06E−10 | 1.18E−10 | >1000x |

### 6.5 Example 5: PBMC Mediated Tumor Cell Killing of Protease Activated ISVD Constructs

In this Example, the following ISVD constructs were tested: T028200194, T028200192 (EGFR ISVD construct) and T028200154, T028200057 (HER2 ISVD construct). ISVD constructs containing a protease cleavable linker (T028200192, T028200057) were pre-treated with Human u-Plasminogen Activator (uPA)/Urokinase (R&D Systems, cat #1310-SE) in D-PBS (Life Technologies-Gibco, cat #14190-250) for 20 hours at room temperature. uPA treatment of masked ISVD constructs with a protease cleavable linker resulted in activated EGFR and HER2 ISVD constructs and are referred to as T028200192_uPA and T028200057_uPA, respectively, in the figures and tables in this Example.

### Impedance-Based Cytotoxicity Assay

The experiments were performed according to the protocol as described in Example 4 using peripheral blood mononuclear cells (PBMCs) as effector cells with an E:T ratio of 10:1. Seeding densities of the target cells are described in Table 9. The data was analyzed at various time points depending on the cell growth of the target cells (as indicated in Table 9) and normalized to the time point that compounds and effector cells were added.

TABLE 9
Overview of seeding density of target
cell lines and time point of analysis.

| | Seeding density/ | Time points | |
|---|---|---|---|
| Cell line | 96-well | of analysis | |
| NCI-H292 | 6.5E+03 | 20-96 | h |
| LoVo | 2.0E+04 | 20-96 | h |
| HCC1954 | 1.0E+04 | 22-90 | h |
| ZR-75-1 | 1.5E+04 | 22-90 | h |
| BT-549 | 1.0E+04 | 20-120 | h |

### Results

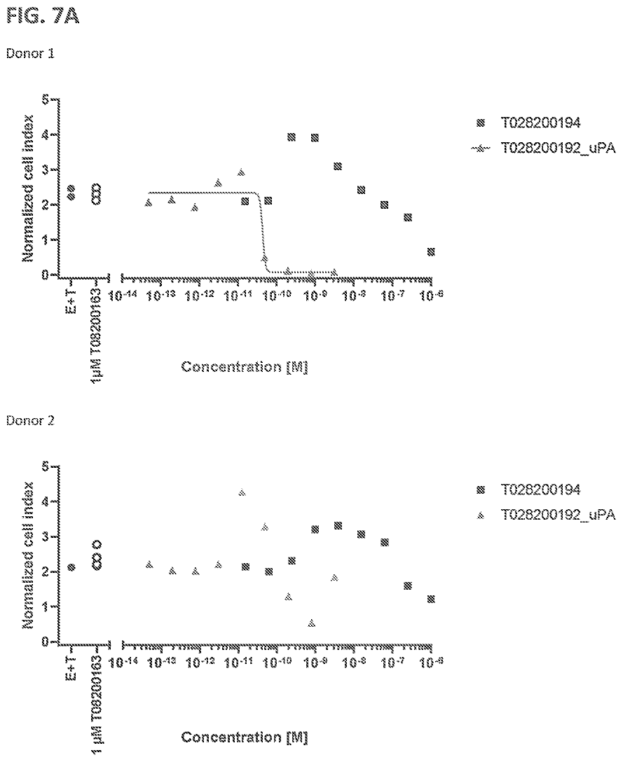

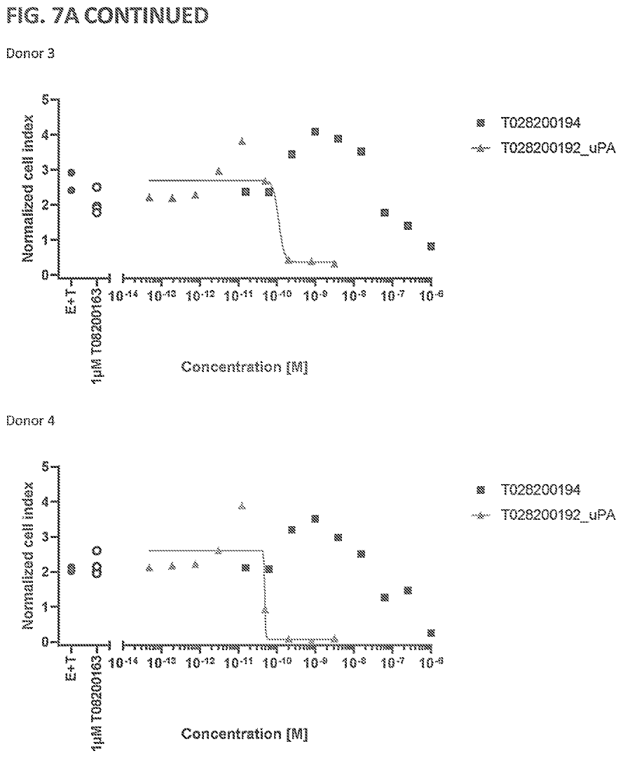

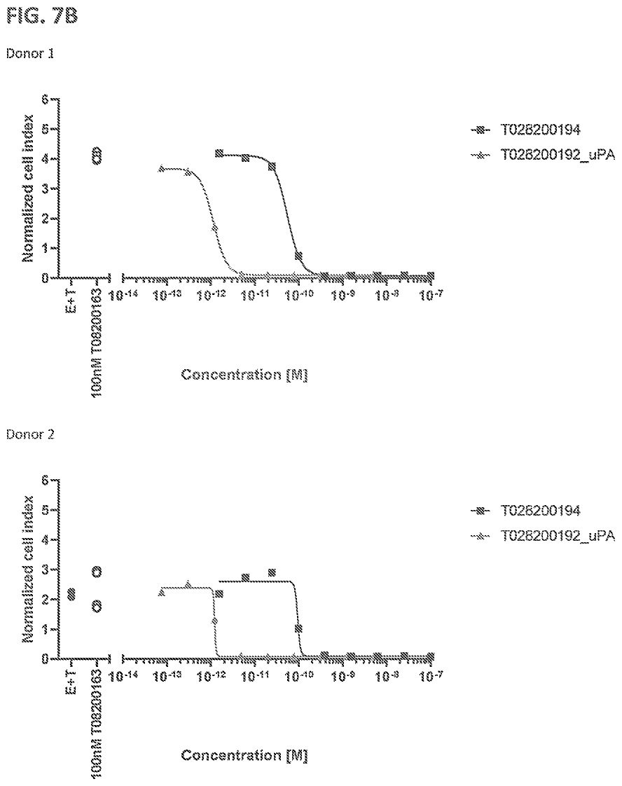

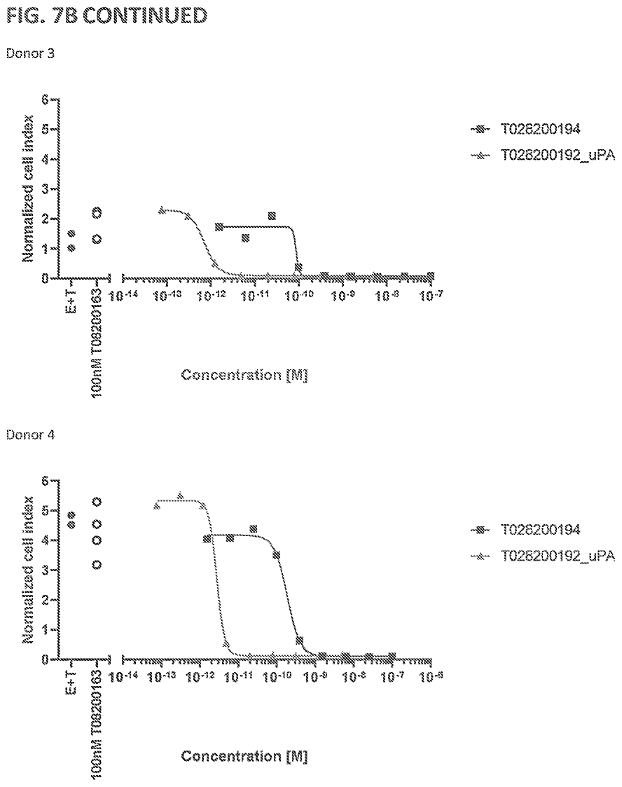

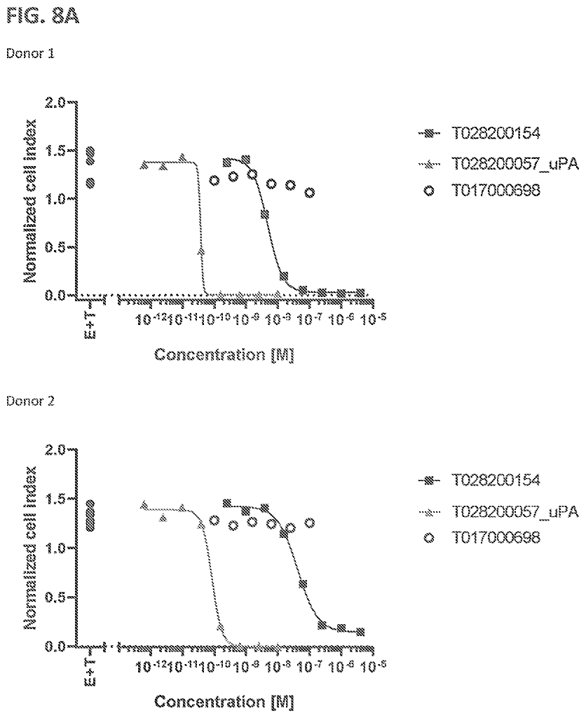

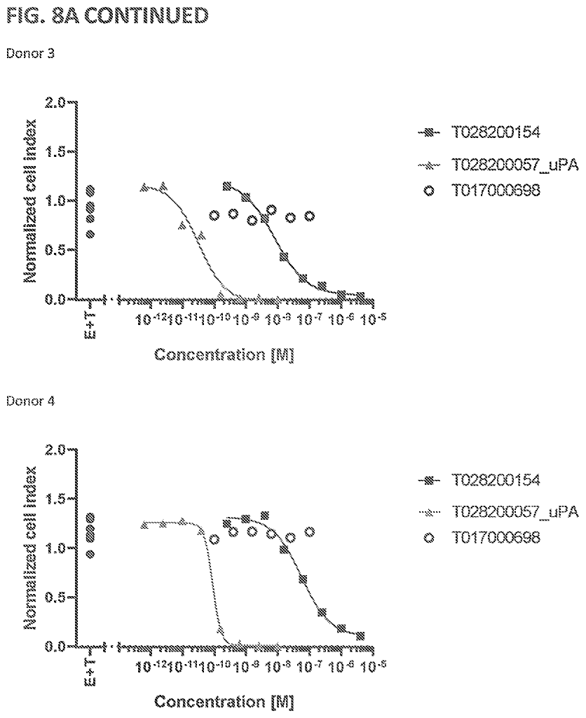

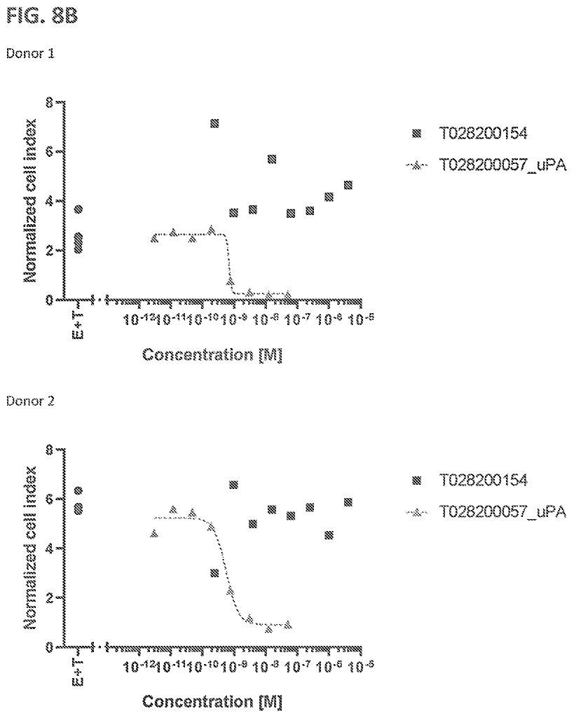

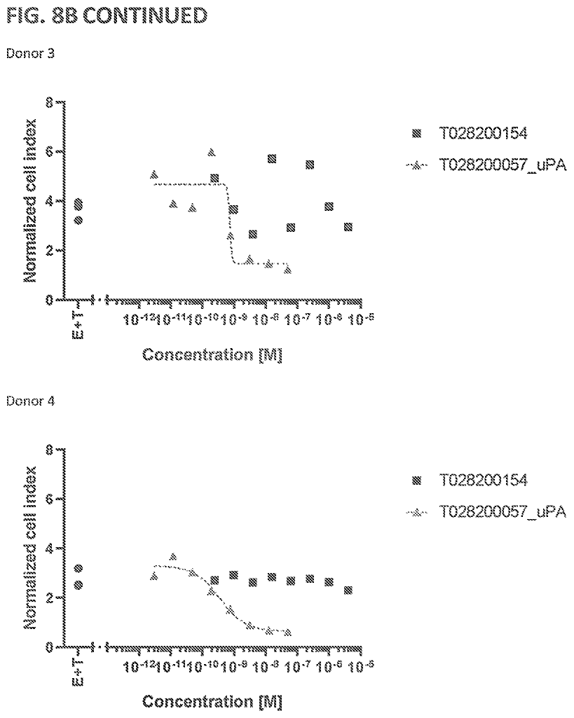

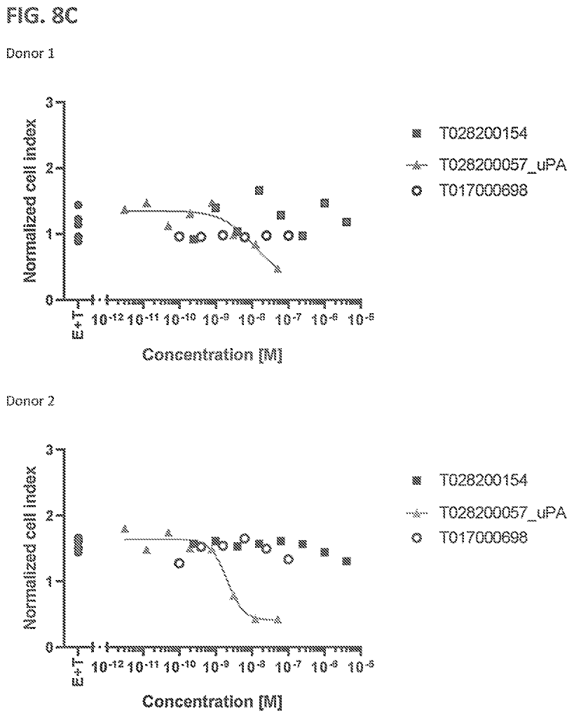

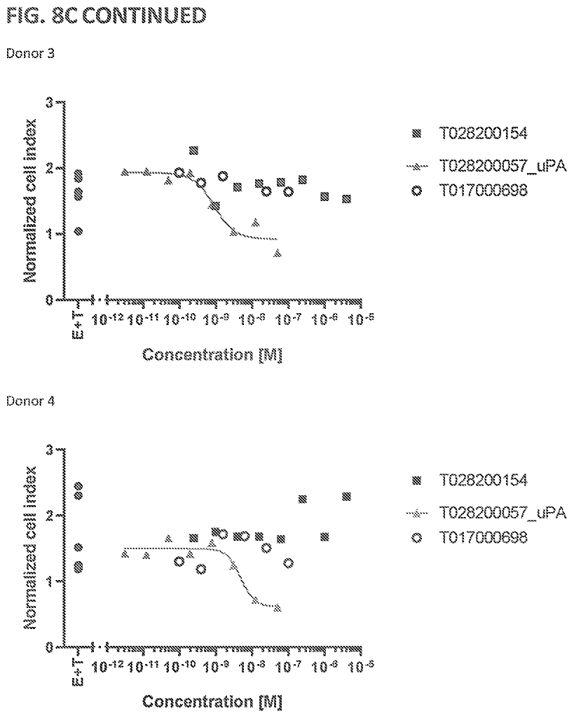

As has been shown in the assay with purified T cells (Example 4), in the assay with PBMCs a strong difference in killing potency was also observed between a masked ISVD construct and a protease activated ISVD construct. Results are consistent between EGFR and HER2 ISVD constructs, for different cell lines and across different PBMC donors (FIG. 7, FIG. 8, Table 10, Table 11). With these results, HSA-mediated masking of an ISVD construct containing ISVD TCR00688 is illustrated with 2 different tumor antigens. Releasing the masking moiety by protease like uPA, results in efficient killing of tumor cell lines by the activated ISVD construct. The fold difference in killing potency between masked and activated ISVD construct is also driven by the expression level of the tumor antigen, especially with HER2 ISVD construct and in lesser extent with EGFR ISVD construct.

TABLE 10
IC50 values and the fold masking for the masked EGFR
ISVD construct and the uPA protease activated EGFR
ISVD construct. Data is confirmed with 4 PBMC donors.

| | T028200194 [IC50, M] | | T028200192_uPA [IC50, M] | | Fold masking | |
|---|---|---|---|---|---|---|
| | Donor 1 | Donor 2 | Donor 1 | Donor 2 | Donor 1 | Donor 2 |
| NCI-H292 | >1.0E−07 | >1.0E−07 | 4.37E−11 | n.a. | >2000x | n.a. |
| LoVo | 5.51E−11 | 9.42E−11 | 1.14E−12 | 1.23E−12 | 48 | >70x |
| | T028200194 [IC50, M] | | T028200192_uPA [IC50, M] | | Fold masking | |
| | Donor 3 | Donor 4 | Donor 3 | Donor 4 | Donor 3 | Donor 4 |
| NCI-H292 | >1.0E−07 | >1.0E−07 | 1.11E−10 | 4.82E−11 | >900x | >2000x |
| LoVo | 8.71E−11 | 1.86E−10 | 7.13E−13 | 2.76E−12 | >120x | 67 |

n.a.—not available

TABLE 11
IC50 values and the fold masking for the masked HER2
ISVD construct and the uPA protease activated HER2
ISVD construct. Data is confirmed with 4 PBMC donors.

| | T028200154 [IC50, M] | | T028200057_uPA [IC50, M] | | Fold masking | |
|---|---|---|---|---|---|---|
| | Donor 1 | Donor 2 | Donor 1 | Donor 2 | Donor 1 | Donor 2 |
| HCC1954 | 4.83E−09 | 4.21E−08 | 3.68E−11 | 8.40E−11 | >100x | 501 |
| ZR-75-1 | No killing | No killing | 7.04E−10 | 5.68E−10 | >1000x | >1000x |
| BT-549 | No killing | No killing | 1.08E−08 | 2.04E−09 | >1000x | >1000x |
| | T028200154 [IC50, M] | | T028200057_uPA [IC50, M] | | Fold masking | |
| | Donor 3 | Donor 4 | Donor 3 | Donor 4 | Donor 3 | Donor 4 |
| HCC1954 | 7.94E−09 | 5.61E−08 | 3.19E−11 | 8.85E−11 | 249 | 633 |
| ZR-75-1 | No killing | No killing | 3.59E−10 | 7.47E−10 | >1000x | >1000x |
| BT-549 | No killing | No killing | 4.83E−09 | 8.89E−10 | >1000x | >1000x |

### 6.6 Example 6: T Cell Activation of Protease Activated ISVD Constructs

In this Example, the following ISVD constructs were tested: T028200194, T028200192 (EGFR ISVD construct) and T028200154, T028200057 (HER2 ISVD construct). ISVD constructs containing a protease cleavable linker (T028200192, T028200057) were pre-treated with Human u-Plasminogen Activator (uPA)/Urokinase (R&D Systems, cat #1310-SE) in D-PBS (Life Technologies-Gibco, cat #14190-250) for 20 hours at room temperature. uPA treatment of masked ISVD constructs with a protease cleavable linker resulted in activated EGFR and HER2 ISVD constructs and are referred to as T028200192_uPA and T028200057_uPA, respectively, in the figures and tables in this Example.

### Flow Cytometry-Based Read-Out for T Cell Activation

Precision activated ISVD constructs were characterized for T cell activation (CD69) in flow cytometry using PBMCs and adherent target cells.

The target cells and PBMCs were harvested 24 hours after adding the serially diluted ISVD construct solutions and effector cells to the target cells. Target and effector cells were resuspended in FACS buffer consisting of D-PBS (Life Technologies-Gibco, cat #14190-094), 2% FBS (Sigma-Aldrich, cat #F7524), and 0.05% NaN3 (Acros Organics, cat #19038), and were transferred to a 96-well V-bottom plate. The cells were diluted in Zombie NIR™ Fixable Viability Kit (Biolegend, cat #423106) in D-PBS and incubated for 15 minutes at room temperature in the dark. The cells were stained in 100 μL antibody cocktail (CD45 (Biolegend, cat #368522), CD4 (Biolegend, cat #317420), CD8 (Biolegend, cat #344712), CD69 (Biolegend, cat #310910)) diluted in FACS buffer and incubated for 30 minutes at 4° C. in the dark. Between each step, the cells were centrifuged twice for 2 minutes at 300 g at 4° C. and washed with 100 μL/well FACS buffer. The cells were diluted in 50 μL FACS buffer and read-out was performed on MACSQuantX (Miltenyi Biotec).

### Results

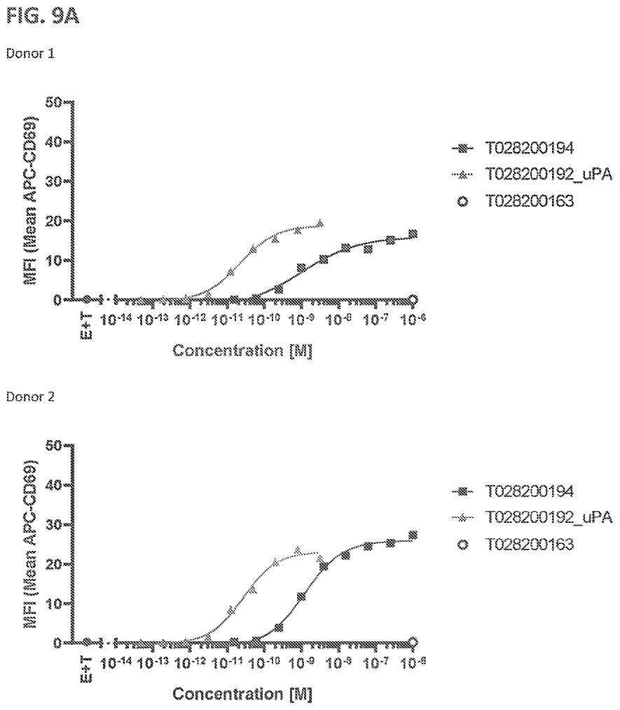

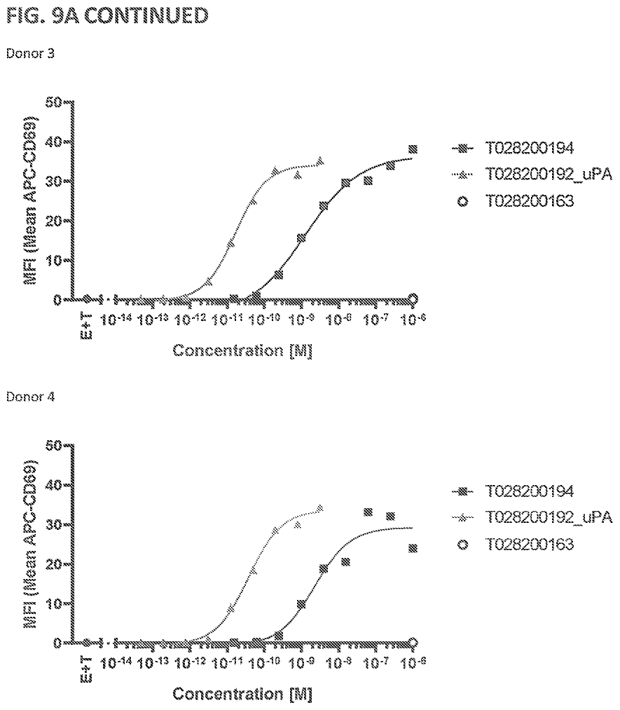

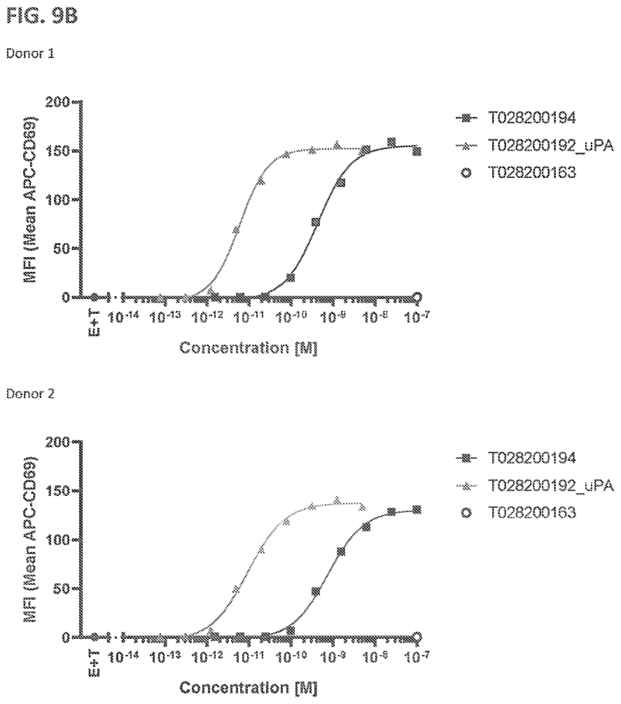

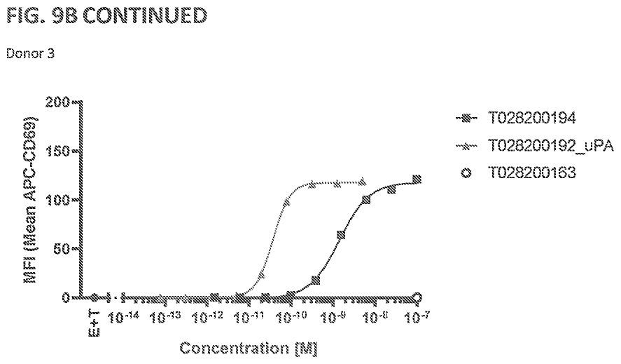

Reduced CD69 expression, which is a marker for T cell activation, on CD3⁺ T cells is noticed for the masked EGFR ISVD construct (T028200194) compared to the uPA activated EGFR ISVD construct (T028200192_uPA, FIG. 9). The fold masking (40-80×) is similar on NCI-H292 and LoVo cells, suggesting that the masking is independent of the EGFR expression levels (Table 12).

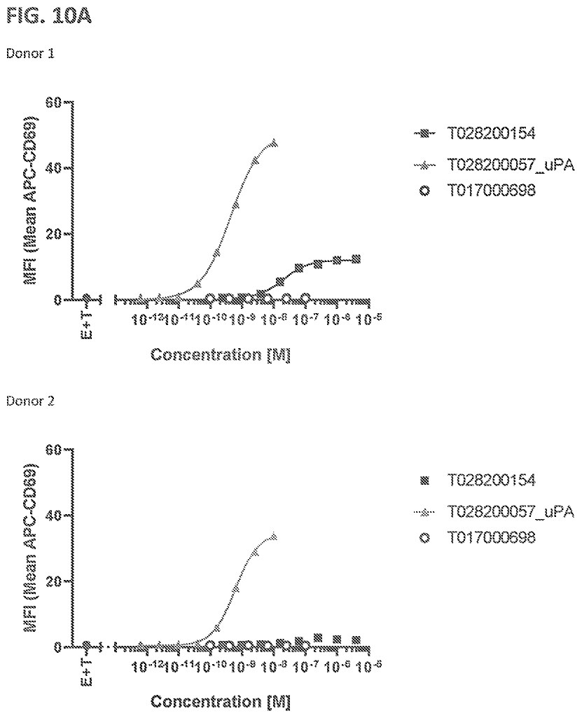

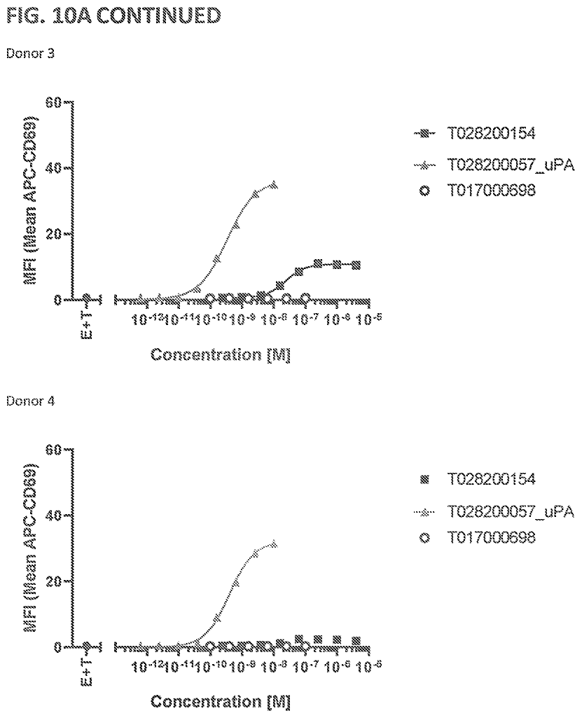

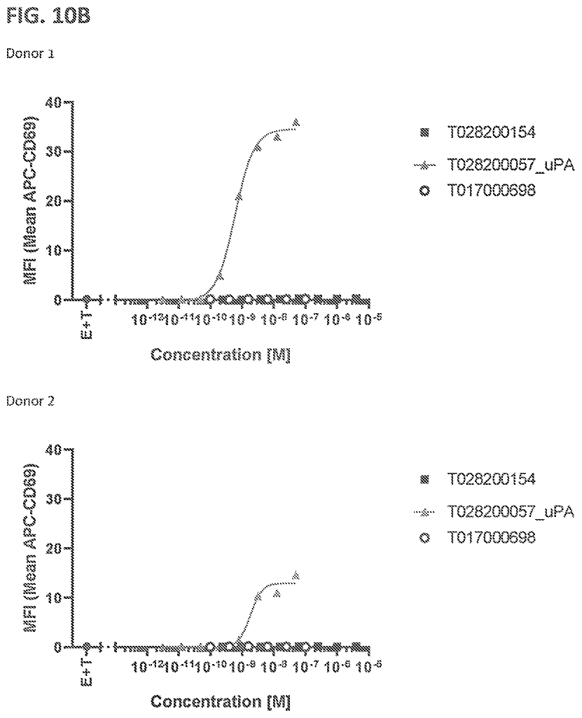

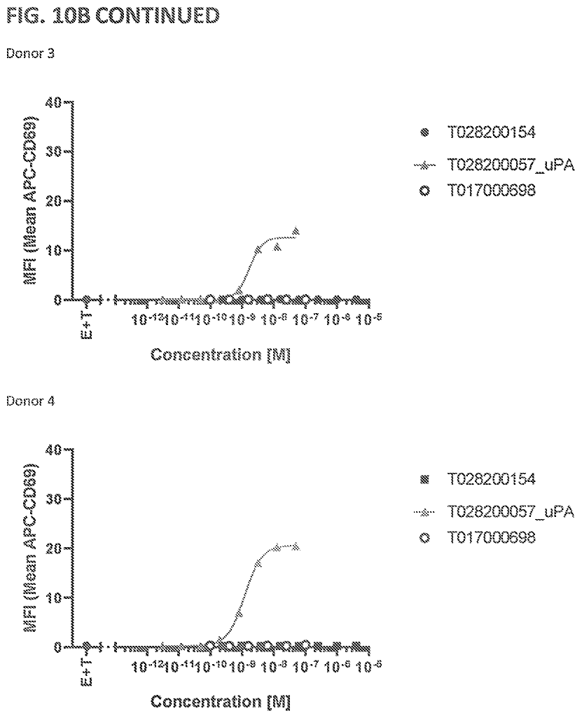

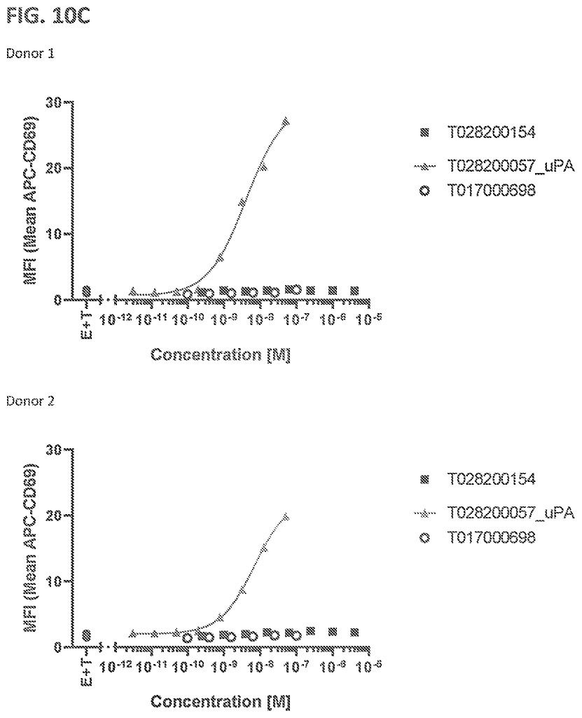

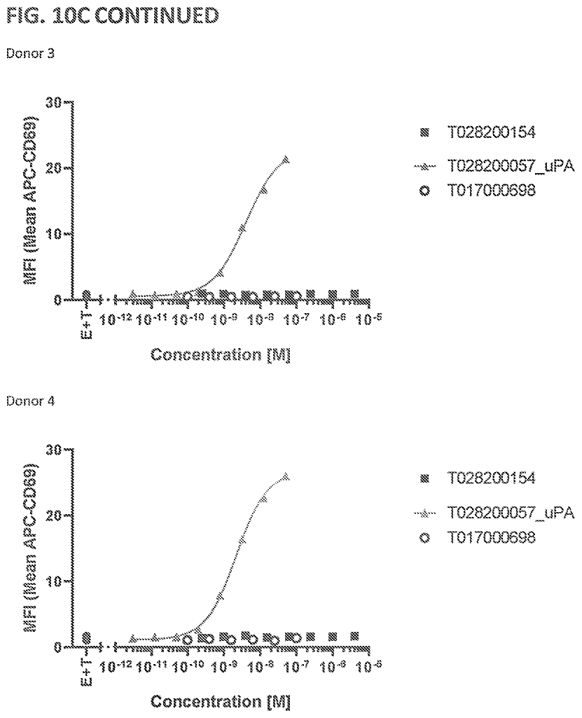

Similarly, reduced CD69 expression is also observed with the masked HER2 ISVD construct (T028200154) compared to the uPA activated HER2 ISVD construct (T028200057_uPA, FIG. 10). The fold masking is 20-70× on HCC1954 cells, while no CD69 expression, and hence no T cell activation, is observed for the masked ISVD construct on ZR-75-1 and BT-549 cells resulting in a 1000-fold difference between the masked and activated HER2 ISVD construct on those cell lines (Table 13).

The results are consistent between 4 different PBMC donors and confirm the cytotoxicity data obtained in Example 4 (FIG. 4, Table 6, FIG. 5, Table 7) and Example 5 (FIG. 7, Table 10, FIG. 8, Table 11). Moreover, HSA-mediated masking of an ISVD construct containing ISVD TCR00688 is illustrated in a T cell activation assay with 2 different tumor antigens. Releasing the masking moiety by protease like uPA, resulted in increased CD69 expression on CD3⁺ T cells induced by the activated ISVD construct.

TABLE 12
EC50 values and fold masking for T cell activation (CD69 expression) of a masked EGFR ISVD
construct and a uPA protease activated EGFR ISVD construct on NCI-H292 and LoVo cells.

| | T028200194 [EC50, M] | | T028200192_uPA [EC50, M] | | | |
|---|---|---|---|---|---|---|
| | (MeanFI CD69-APC) | | (MeanFI CD69-APC) | | Fold masking | |
| | Donor 1 | Donor 2 | Donor 1 | Donor 2 | Donor 1 | Donor 2 |
| NCI-H292 | 9.69E−10 | 1.20E−09 | 2.23E−11 | 2.69E−11 | 44 | 44 |
| | T028200194 [EC50, M] | | T028200192_uPA [EC50, M] | | | |
| | (MeanFI CD69-APC) | | (MeanFI CD69-APC) | | Fold masking | |
| | Donor 3 | Donor 4 | Donor 3 | Donor 4 | Donor 3 | Donor 4 |
| NCI-H292 | 1.28E−09 | 2.23E−09 | 1.69E−11 | 3.75E−11 | 76 | 60 |

| | T028200194 [EC50, M] | | | T028200192_uPA [EC50, M] | | | | | |
|---|---|---|---|---|---|---|---|---|---|
| | (MeanFI CD69-APC) | | | (MeanFI CD69-APC) | | | Fold masking | | |
| | Donor 1 | Donor 2 | Donor 3 | Donor 1 | Donor 2 | Donor 3 | Donor 1 | Donor 2 | Donor 3 |
| LoVo | 4.35E−10 | 7.53E−10 | 1.42E−09 | 5.97E−12 | 9.33E−12 | 3.68E−11 | 73 | 81 | 38 |

TABLE 13
EC50 values and fold masking for T cell activation (CD69 expression)
of a masked HER2 ISVD construct and uPA protease activated HER2
ISVD construct on HCC1954, ZR-75-1 and BT-549 cells.

| | T028200154 [EC50, M] | | T028200057_uPA [EC50, M] | | Fold masking | |
|---|---|---|---|---|---|---|
| | Donor 1 | Donor 2 | Donor 1 | Donor 2 | Donor 1 | Donor 2 |
| HCC1954 | 2.04E−08 | >1.0E−08 | 4.35E−10 | 6.05E−10 | 47 | >20x |
| ZR-75-1 | No TCA | No TCA | 5.98E−10 | 1.82E−09 | >1000x | >1000x |
| BT-549 | No TCA | No TCA | 4.03E−09 | 6.88E−09 | >1000x | >1000x |
| | T028200154 [EC50, M] | | T028200057_uPA [EC50, M] | | Fold masking | |
| | Donor 3 | Donor 4 | Donor 3 | Donor 4 | Donor 3 | Donor 4 |
| HCC1954 | 2.40E−08 | >1.0E−08 | 3.35E−10 | 3.99E−10 | 72 | >20x |
| ZR-75-1 | No TCA | No TCA | 1.19E−09 | 1.68E−09 | >1000x | >1000x |
| BT-549 | No TCA | No TCA | 2.19E−09 | 4.00E−09 | >1000x | >1000x |

No TCA: no T cell activation (=no CD69 expression)

### 6.7 Example 7: PBMC Cytokine Secretion Induced by Protease Activated ISVD Constructs

In this Example, the following ISVD constructs were tested: T028200194, T028200192 (EGFR ISVD construct) and T028200154, T028200057 (HER2 ISVD construct). ISVD constructs containing a protease cleavable linker (T028200192, T028200057) were pre-treated with Human u-Plasminogen Activator (uPA)/Urokinase (R&D Systems, cat #1310-SE) in D-PBS (Life Technologies-Gibco, cat #14190-250) for 20 hours at room temperature. uPA treatment of masked ISVD constructs with a protease cleavable linker resulted in activated EGFR and HER2 ISVD constructs and are referred to as T028200192_uPA and T028200057_uPA, respectively, in the figures and tables in this Example.

### Bead-Based Multiplex Assay for Cytokine Read-Out

Protease activated ISVD constructs were characterized for induction of interleukin-2 (IL-2), interleukin-6 (IL-6), Tumor Necrosis Factor Alpha (TNF-α) and Interferon gamma (IFN-γ) secretion in a bead-based multiplex assay using human PBMCs and adherent target cells. Supernatant was collected 24 hours after adding the serially diluted ISVD construct solutions and effector cells to the target cells.

Each bead stock was diluted in Bioplex assay buffer (Bio-Rad, cat #9723892) and 50 μL was added to a 96-well V-bottom plate. Subsequently, 50 μL diluted supernatant, standard dilutions or blanks were added to the 96-well V-bottom plate. The plate was covered and incubated for 30 minutes at 850 rpm at room temperature. Cytokine binding to the beads was evaluated using a detection antibody mix (Bio-Rad, cat #1207919, 30 minutes at 850 rpm at room temperature) followed by streptavidin-PE (Bio-Rad, cat #171304501, 10 minutes at 850 rpm at room temperature). Between each step, the beads were washed three times with Bio-Plex wash buffer (Bio-Rad, cat #171304040) and a magnet was placed for approximately one minute to remove the wash buffer. Read-out was performed on Luminex FLEXMAP 3D. Cytokine quantification was calculated using the Bio-Plex Pro Human Cytokine Screening Panel Standards (Bio-Rad, cat #12007919).

### Results

A higher EC50 is noticed for various cytokines (IL-2, IL-6, IFN-γ, TNF-α) in case of a masked EGFR ISVD construct (T028200194) compared to a uPA protease activated ISVD construct (T028200192_uPA) on NCI-H292 and LoVo cells (Table 14). This shift in potency is most pronounced with NCI-H292 cells, which is in line with PBMC mediated killing and T cell activation data as obtained in Example 5 and Example 6.

Similar results were obtained with the HER2 ISVD construct: no (ZR-75-1, BT-549) to very low (HCC1954) cytokine secretion is noticed with the masked compound (T028200154), while the active HER2 ISVD construct (T028200057_uPA) results in IL-2, IL-6, TNF-α and IFN-γ secretion (Table 15 Error! Reference source not found.). On BT-549 tumor cells, incomplete dose-response curves and low cytokine secretion top levels were obtained with the active HER2 ISVD construct. This correlates with the lower killing efficacy of the active HER2 ISVD construct (FIG. 5E, FIG. 8C) on these cells. Similarly, to EGFR ISVD construct data, the cytokine secretion results confirm the PBMC mediated killing and T cell activation data obtained in Example 5 and Example 6 for the HER2 ISVD construct.

These results illustrate the HSA-mediated masking of EGFR and HER2 ISVD construct containing ISVD TCR00688 in cytokine secretion assay. By releasing the masking moiety by protease like uPA, the active ISVD construct can induce cytokine secretion. This is illustrated using EGFR and HER tumor cell lines with different EGFR or HER2, respectively, expression levels. Moreover, the results have been confirmed with 4 different PBMC donors.

TABLE 14
PBMC - tumor cell assay. EC50 values for induction of various
cytokines by a masked EGFR ISVD construct (T028200194) and uPA
protease activated EGFR ISVD construct (T028200192_uPA).
(a) NCI-H292 cells, (b) LoVo cells and (c) BT-549 cells.
(a) NCI-H292 cells

| | IL-6 | | | |
|---|---|---|---|---|
| | Donor 1 | | Donor 2 | |
| ID | EC50 [M] | Top (pg/mL) | EC50 [M] | Top (pg/mL) |
| T028200194 | 1.32E−09 | 3536 | 4.86E−09 | 2395 |
| T028200192_uPA | 1.22E−11 | 1832 | 4.32E−11 | 2619 |
| | IL-6 | | | |
| | Donor 3 | | Donor 4 | |
| ID | EC50 [M] | Top (pg/mL) | EC50 [M] | Top (pg/mL) |
| T028200194 | 1.01E−09 | 4223 | 4.86E−09 | 2395 |
| T028200192_uPA | 1.34E−11 | 4577 | 4.32E−11 | 2619 |
| | IFN-γ | | | |
| | Donor 1 | | Donor 2 | |
| ID | EC50 [M] | Top (pg/mL) | EC50 [M] | Top (pg/mL) |
| T028200194 | 3.41E−09 | 42 | 7.00E−09 | 18 |
| T028200192_uPA | 7.82E−11 | 50 | =LL | =LL |
| | IFN-γ | | | |
| | Donor 3 | | Donor 4 | |
| ID | EC50 [M] | Top (pg/mL) | EC50 [M] | Top (pg/mL) |
| T028200194 | 1.37E−08 | 649 | n.a. | n.a. |
| T028200192_uPA | 3.86E−11 | 738 | 8.07E−11 | 867 |
| n.a.: not available | | | | |
| | TNF-α | | | |
| | Donor 1 | | Donor 2 | |
| ID | EC50 [M] | Top (pg/mL) | EC50 [M] | Top (pg/mL) |
| T028200194 | 4.47E−09 | 15134 | 2.66E−08 | 11804 |
| T028200192_uPA | 1.25E−11 | 4667 | 6.40E−11 | 4929 |
| | TNF-α | | | |
| | Donor 3 | | Donor 4 | |
| ID | EC50 [M] | Top (pg/mL) | EC50 [M] | Top (pg/mL) |
| T028200194 | 2.66E−09 | 26733 | 5.62E−09 | 20822 |
| T028200192_uPA | 1.47E−11 | 16634 | 2.21E−11 | 12091 |

(b) LoVo cells

| | IL-6 | | | | | |
|---|---|---|---|---|---|---|
| | Donor 1 | | Donor 2 | | Donor 3 | |
| ID | EC50 [M] | Top (pg/mL) | EC50 [M] | Top (pg/mL) | EC50 [M] | Top (pg/mL) |
| T028200194 | 3.10E−10 | 399 | 6.95E−10 | 342 | <LL | <LL |
| T028200192_uPA | 1.57E−11 | 392 | 2.70E−11 | 449 | <LL | <LL |
| | IFN-γ | | | | | |
| | Donor 1 | | Donor 2 | | Donor 3 | |
| ID | EC50 [M] | Top (pg/mL) | EC50 [M] | Top (pg/mL) | EC50 [M] | Top (pg/mL) |
| T028200194 | 5.26E−09 | 139 | <LL | <LL | <LL | <LL |
| T028200192_uPA | 5.19E−11 | 183 | 5.32E−11 | 63 | <LL | <LL |
| | TNF-α | | | | | |
| | Donor 1 | | Donor 2 | | Donor 3 | |
| ID | EC50 [M] | Top (pg/mL) | EC50 [M] | Top(pg/mL) | EC50 [M] | Top (pg/mL) |
| T028200194 | 1.16E−09 | 14862 | 1.14E−09 | 12042 | =<LL | =<LL |
| T028200192_uPA | 2.13E−11 | 10797 | 2.68E−11 | 11452 | 7.27E−11 | 3364 |

| | IL-2 | | | | | |
|---|---|---|---|---|---|---|
| | Donor 1 | | Donor 2 | | Donor 3 | |
| ID | EC50 [M] | Top (pg/mL) | EC50 [M] | Top (pg/mL) | EC50 [M] | Top (pg/mL) |
| T028200194 | 9.93E−10 | 6061 | 1.14E−08 | 3374 | 4.60E−09 | 1326 |
| T028200192_uPA | 3.51E−11 | 3793 | 3.81E−11 | 1751 | 8.96E−11 | 665 |

Top values were measured from a starting concentration of 1.00E−07M (T028200194) and 5.00E−09M (T028200192_uPA).

(c) BT-549 cells

| | IL-6 | | | |
|---|---|---|---|---|
| | Donor 1 | | Donor 2 | |
| ID | EC50 [M] | Top (pg/mL) | EC50 [M] | Top (pg/mL) |
| T028200154 | iDRC | >1400 | =LL | =LL |
| T028200057_uPA | iDRC | >=1714 | iDRC | >=1011 |
| | IL-6 | | | |
| | Donor 3 | | Donor 4 | |
| ID | EC50 [M] | Top (pg/mL) | EC50 [M] | Top (pg/mL) |
| T028200154 | =LL | =LL | =LL | =LL |
| T028200057_uPA | iDRC | >=2227 | iDRC | >=1561 |
| | IFN-γ | | | |
| | Donor 1 | | Donor 2 | |
| ID | EC50 [M] | Top (pg/mL) | EC50 [M] | Top (pg/mL) |
| T028200154 | <LL | <LL | 7.00E−09 | 18 |
| T028200057_uPA | <LL | <LL | =LL | =LL |
| | IFN-γ | | | |
| | Donor 3 | | Donor 4 | |
| ID | EC50 [M] | Top (pg/mL) | EC50 [M] | Top (pg/mL) |
| T028200154 | <LL | <LL | <LL | <LL |
| T028200057_uPA | iDRC | >=59 | iDRC | >=106 |
| | TNF-α | | | |
| | Donor 1 | | Donor 2 | |
| ID | EC50 [M] | Top (pg/mL) | EC50 [M] | Top (pg/mL) |
| T028200154 | =LL | =LL | =LL | =LL |
| T028200057_uPA | iDRC | >=1589 | iDRC | >=470 |
| | TNF-α | | | |
| | Donor 3 | | Donor 4 | |
| ID | EC50 [M] | Top (pg/mL) | EC50 [M] | Top (pg/mL) |
| T028200154 | =LL | =LL | =LL | =LL |
| T028200057_uPA | iDRC | >=1177 | iDRC | >=1008 |
| | IL-2 | | | |
| | Donor 1 | | Donor 2 | |
| ID | EC50 [M] | Top (pg/mL) | EC50 [M] | Top (pg/mL) |
| T028200154 | <LL | <LL | <LL | <LL |
| T028200057_uPA | iDRC | >=36 | iDRC | >=50 |
| | IL-2 | | | |
| | Donor 3 | | Donor 4 | |
| ID | EC50 [M] | Top (pg/mL) | EC50 [M] | Top (pg/mL) |
| T028200154 | <LL | <LL | <LL | <LL |
| T028200057_uPA | iDRC | >=128 | iDRC | >=89 |

iDRC: incomplete dose-response curve, LL: Lower Limit.
Top values were measured from a starting concentration of 4.00E−06M (T028200154) and 5.00E−08M (T028200057_uPA).

TABLE 15
EC50 values for induction of various cytokines by a masked HER2 ISVD construct (T028200154) and a uPA protease
activated HER2 ISVD construct (T028200057_uPA). (a) HCC1954 cells, (b) ZR-75-1 cells and (c) BT-549 cells.
(a) HCC1954 cells

| | IL-6 | | | |
|---|---|---|---|---|
| | Donor 1 | | Donor 2 | |
| ID | EC50 [M] | Top | EC50 [M] | Top |
| T028200154 | >1.00E−06 | >=1054 | =<LL | =<LL |
| T028200057_uPA | 5.71E−09 | 17295 | 2.44E−09 | 3266 |
| | IL-6 | | | |
| | Donor 3 | | Donor 4 | |
| ID | EC50 [M] | Top | EC50 [M] | Top |
| T028200154 | >1.00E−07 | >=107 | <LL | <LL |
| T028200057_uPA | 6.19E−10 | 4621 | 7.11E−10 | 2266 |

| | IFN-γ | | | |
|---|---|---|---|---|
| | Donor 1 | | Donor 2 | |
| ID | EC50 [M] | Top | EC50 [M] | Top |
| T028200154 | <LL | <LL | =<LL | =<LL |
| T028200057_uPA | n.a. | n.a. | 6.01E−09 | 507 |
| | IFN-γ | | | |
| | Donor 3 | | Donor 4 | |
| ID | EC50 [M] | Top | EC50 [M] | Top |
| T028200154 | <LL | <LL | <LL | <LL |
| T028200057_uPA | 1.02E−09 | 527 | 4.70E−10 | 133 |

| | TNF-α | | | |
|---|---|---|---|---|
| | Donor 1 | | Donor 2 | |
| ID | EC50 [M] | Top (pg/mL) | EC50 [M] | Top (pg/mL) |
| T028200154 | >1.00E−08 | >=8518 | <LL | <LL |
| T028200057_uPA | 1.90E−09 | 61588 | 2.82E−09 | 16680 |

| | TNF-α | | | |
|---|---|---|---|---|
| | Donor 3 | | Donor 4 | |
| ID | EC50 [M] | Top (pg/mL) | EC50 [M] | Top (pg/mL) |
| T028200154 | =<LL | =<LL | =<LL | =<LL |
| T028200057_uPA | 3.99E−10 | 21323 | 2.72E−10 | 19764 |

| | IL-2 | | | |
|---|---|---|---|---|
| | Donor 1 | | Donor 2 | |
| ID | EC50 [M] | Top (pg/mL) | EC50 [M] | Top (pg/mL) |
| T028200154 | >1.00E−06 | >=1054 | =<LL | =<LL |
| T028200057_uPA | 5.71E−09 | 17295 | 2.44E−09 | 3266 |

| | IL-2 | | | |
|---|---|---|---|---|
| | Donor 3 | | Donor 4 | |
| ID | EC50 [M] | Top (pg/mL) | EC50 [M] | Top (pg/mL) |
| T028200154 | >1.00E−08 | >=392 | >1.00E−08 | >=133 |
| T028200057_uPA | 1.42E−09 | 3679 | 5.56E−10 | 1468 |

LL: Lower Limit. n.a.: not available.
Top values were measured from a starting concentration of 4.00E−06M (T028200154) and 1.00E−08M (T028200057_uPA).

(b) ZR-75-1 cells

| | IL-6 | | | |
|---|---|---|---|---|
| | Donor 1 | | Donor 2 | |
| ID | EC50 [M] | Top (pg/mL) | EC50 [M] | Top (pg/mL) |
| T028200154 | <LL | <LL | <LL | <LL |
| T028200057_uPA | 1.31E−09 | 600 | =LL | =LL |
| | IL-6 | | | |
| | Donor 3 | | Donor 4 | |
| ID | EC50 [M] | Top (pg/mL) | EC50 [M] | Top (pg/mL) |
| T028200154 | =<LL | =<LL | =<LL | =<LL |
| T028200057_uPA | 1.45E−09 | 23 | 3.36E−09 | 284 |
| | IFN-γ | | | |
| | Donor 1 | | Donor 2 | |
| ID | EC50 [M] | Top (pg/mL) | EC50 [M] | Top (pg/mL) |
| T028200154 | <LL | <LL | <LL | <LL |
| T028200057_uPA | 4.23E−09 | 1365 | 1.11E−08 | 271 |
| | IFN-γ | | | |
| | Donor 3 | | Donor 4 | |
| ID | EC50 [M] | Top (pg/mL) | EC50 [M] | Top (pg/mL) |
| T028200154 | <LL | <LL | <LL | <LL |
| T028200057_uPA | <LL | <LL | 2.74E−09 | 199 |
| | TNF-α | | | |
| | Donor 1 | | Donor 2 | |
| ID | EC50 [M] | Top (pg/mL) | EC50 [M] | Top (pg/mL) |
| T028200154 | <LL | <LL | <LL | <LL |
| T028200057_uPA | 1.06E−09 | 18318 | 2.12E−09 | 7338 |
| | TNF-α | | | |
| | Donor 3 | | Donor 4 | |
| ID | EC50 [M] | Top (pg/mL) | EC50 [M] | Top (pg/mL) |
| T028200154 | =<LL | =<LL | =<LL | =<LL |
| T028200057_uPA | 2.23E−09 | 6335 | 1.07E−09 | 6955 |
| | IL-2 | | | |
| | Donor 1 | | Donor 2 | |
| ID | EC50 [M] | Top (pg/mL) | EC50 [M] | Top (pg/mL) |
| T028200154 | <LL | <LL | <LL | <LL |
| T028200057_uPA | 1.61E−09 | 1199 | 4.37E−09 | 669 |

| | IL-2 | | | |
|---|---|---|---|---|
| | Donor 3 | | Donor 4 | |
| ID | EC50 [M] | Top (pg/mL) | EC50 [M] | Top (pg/mL) |
| T028200154 | <LL | <LL | <LL | <LL |
| T028200057_uPA | 2.41E−09 | 474 | 1.79E−09 | 881 |

LL: Lower Limit.
Top values were measured from a starting concentration of 4.00E−06M (T028200154) and 5.00E−08M (T028200057_uPA).

(c) BT-549 cells

| | IL-6 | | | |
|---|---|---|---|---|
| | Donor 1 | | Donor 2 | |
| ID | EC50 [M] | Top (pg/mL) | EC50 [M] | Top (pg/mL) |
| T028200154 | iDRC | >1400 | =LL | =LL |
| T028200057_uPA | iDRC | >=1714 | iDRC | >=1011 |
| | IL-6 | | | |
| | Donor 3 | | Donor 4 | |
| ID | EC50 [M] | Top (pg/mL) | EC50 [M] | Top (pg/mL) |
| T028200154 | =LL | =LL | =LL | =LL |
| T028200057_uPA | iDRC | >=2227 | iDRC | >=1561 |
| | IFN-γ | | | |
| | Donor 1 | | Donor 2 | |
| ID | EC50 [M] | Top (pg/mL) | EC50 [M] | Top (pg/mL) |
| T028200154 | <LL | <LL | <LL | <LL |
| T028200057_uPA | <LL | <LL | <LL | <LL |
| | IFN-γ | | | |
| | Donor 3 | | Donor 4 | |
| ID | EC50 [M] | Top (pg/mL) | EC50 [M] | Top (pg/mL) |
| T028200154 | <LL | <LL | <LL | <LL |
| T028200057_uPA | iDRC | >=59 | iDRC | >=106 |
| | TNF-α | | | |
| | Donor 1 | | Donor 2 | |
| ID | EC50 [M] | Top (pg/mL) | EC50 [M] | Top (pg/mL) |
| T028200154 | =LL | =LL | =LL | =LL |
| T028200057_uPA | iDRC | >=1589 | iDRC | >=470 |
| | TNF-α | | | |
| | Donor 3 | | Donor 4 | |
| ID | EC50 [M] | Top (pg/mL) | EC50 [M] | Top (pg/mL) |
| T028200154 | =LL | =LL | =LL | =LL |
| T028200057_uPA | iDRC | >=1177 | iDRC | >=1008 |
| | IL-2 | | | |
| | Donor 1 | | Donor 2 | |
| ID | EC50 [M] | Top (pg/mL) | EC50 [M] | Top (pg/mL) |
| T028200154 | <LL | <LL | <LL | <LL |
| T028200057_uPA | iDRC | >=36 | iDRC | >=50 |
| | IL-2 | | | |
| | Donor 3 | | Donor 4 | |
| ID | EC50 [M] | Top (pg/mL) | EC50 [M] | Top (pg/mL) |
| T028200154 | <LL | <LL | <LL | <LL |
| T028200057_uPA | iDRC | >=128 | iDRC | >=89 |

iDRC: incomplete dose-response curve. LL: Lower Limit.
Top values were measured from a starting concentration of 4.00E−06M (T028200154) and 5.00E−08M (T028200057_uPA).

### 6.8 Example 8: In Vitro, Ex Vivo and In Vivo Activation of ISVD Constructs

Multiple experiments were set up to confirm the protease expression and activity in tumor cells and to validate the protease mediated activation of masked ISVD construct by these proteases. Different in vitro assays (Clariostar, ELISA, Western Blot) were explored to assess protease expression and activity in cell cultures, while protease activity in tissues was explored by MALDI mass spectrometry imaging (MALDI-MSI) and 2D-LC/MS. MALDI-MSI and 2D-LC/MS was applied on ex vivo treated tissues and on tissues from mice treated with an ISVD construct with a protease cleavable linker (T028200192) and an ISVD construct with a non-cleavable linker (T028200194).

### 6.8.1 In Vitro Protease Activity Measurement

### 6.8.1.1 Determination of Protease Activity or Expression in Clariostar, ELISA and Western Blot

Cell culture medium from cultured cell lines was collected after 4 or 7 days and centrifuged at 1000 g for 20 minutes at room temperature (i.e., conditioned medium). The conditioned medium was incubated with Peptide: Z-Gly-Gly-Arg 7-amido-4-methylcoumarin hydrochloride (Bachem, cat #4002155,0025) or Boc-QAR-AMC Fluorogenic Peptide Substrate (R&D Systems, cat #ES014)—substrates for uPA and matriptase, respectively. The conditioned medium/substrate solution was readout on Clariostar (30 minutes with 1 minute interval; 6 hours with 5 minutes interval) to determine in vitro protease activity. The analysis was performed in the MARS data analysis Software v3.41 to calculate the slope in RFU/min.

Next, uPA secretion in the conditioned medium was confirmed in ELISA using the Human PLAU/Urokinase-type Plasminogen Activator ELISA Kit (Sigma-Aldrich, cat #RAB0555-1KT) and the Human U-Plasminogen Activator ELISA (Abcam, cat #ab226904). Several dilutions of the conditioned media were tested, and the assay was performed according to the manufacturers' datasheet.

Target cell lines were incubated for 7 days with conditioned/fresh culture medium (1:1 ratio). At day 7, T028200057 was spiked into cell cultures (final concentration=0.5 mg/ml or 0.1 mg/mL) and incubated 1 and 2 days at 37° C. The cell culture medium was harvested, centrifuged at 1000 g for 20 minutes at room temperature and tested in Western Blot analysis. A rabbit polyclonal antibody targeting TCR VHH (Sanofi, in-house) was used to distinguish intact vs cleaved T028200057.

### 6.8.1.2 Flow Cytometry-Based Read-Out for Matriptase Expression

The target cells were harvested when 80-90% confluency was obtained. The cells were resuspended in FACS buffer, consisting of D-PBS (Life Technologies-Gibco, cat #14190-094), 2% FBS (Sigma-Aldrich, cat #F7524), and 0.05% NaN3 (Acros Organics, cat #19038), and were transferred to a 96-well V-bottom plate. The cells were fixated with fixation buffer (BioLegend, cat #420801) for 20 minutes in the dark. The cells were stained in 100 μL antibody solution (Human Matriptase/ST14 Catalytic Domain Alexa Fluor® 488-conjugated, R&D Systems, cat #IC3946G) diluted in FACS buffer and incubated for 30 minutes at 4° C. in the dark. Between each step, the cells were centrifuged twice for 2 minutes at 300 g at 4° C. and washed with 100 μL/well FACS buffer. The cells were diluted in 50 μL FACS buffer and read-out was performed on MACSQuantX (Miltenyi Biotec).

### 6.8.1.3 Immunohistochemistry Method—Matriptase Staining on NCI-H292 and HCC1954 Cells

The cells were cultured on glass chamber slides until confluent, washed with PBS, fixed with neutral buffered formalin (Sigma, cat #HT5012) for 10 minutes at room temperature (RT) and frozen at −80° C. until staining. On the day of staining, the slides were thawed for at least 30 minutes at RT and washed with PBS/0.05% Tween20. The cells were blocked with 10% rabbit serum for 20 minutes at RT. After washing with PBS/0.05% Tween20, endogenous biotin in the cells was inactivated with the Avidin/Biotin Blocking Kit (VectorLabs, cat #SP-2001) according to the kit instructions. After a wash with PBS/0.05% Tween20, the cell slides were incubated 1 hour at RT with 10 μg/mL Human Matriptase/ST14 Catalytic Domain Antibody (R&DSystems, cat #AF3946). After washing with PBS/0.05% Tween20, the cell slides were incubated with an HRP-labeled rabbit anti-sheep IgG H&L (Abcam, cat #ab97130) for 1 hour at RT. After washing, the sections were stained with DAB (VectorLabs, cat #SK-4105) for 10 minutes at RT. The chromogenic reaction was stopped by washing with MilliQ water and nuclei were stained with hematoxylin (Sigma, cat #51275) counterstain for 2 minutes at RT. After washing with MilliQ water, the cell slides were mounted using aqueous mounting medium (VectorLabs, cat #H-5000).

### 6.8.1.4 Results of the In Vitro Protease Activity Measurement

Matriptase and uPA protease expression and activity was confirmed in HCC1954 and NCI-H292 cells using different measurement methods as illustrated in Table 16, suggesting that precision activated ISVD constructs with a protease cleavable linker can be cleaved by proteases secreted by these cells. These results supported the evaluation of precision activated EGFR and HER2 ISVD constructs in vivo with NCI-H292 and HCC1954 xenograft mouse models, respectively.

TABLE 16
uPA and matriptase expression and activity
for HCC1954 and NCI-H292 cells.

| | ELISA | Clariostar | | FACS | IHC | Western |
|---|---|---|---|---|---|---|
| | uPA | uPA | Matriptase | Matriptase | Matriptase | Blot |
| HCC1954 | ++ | ++ | + | +/− | +/− | + |
| NCI-H292 | + | + | ++ | + | + | +/− |
| Negative | − | − | − | − | − | − |
| control | | | | | | |

### 6.8.2 Ex Vivo and In Vivo Assessment of Protease Cleavage of ISVD Constructs Using MALDI-MSI and 2D-LC/MS

### 6.8.2.1 Tissue Collection

For the ex vivo evaluation of protease activated ISVD construct, tumors and livers were collected from animals that were not engrafted with human T cells and not administrated with treatments (non-randomized animals in PK and efficacy studies). Samples were immediately frozen in liquid nitrogen (snap-frozen samples) and kept at −80° C.

For the in vivo evaluation of the tissues of the treated mice, animals were treated with compounds T028200194 or T028200192 via a single intravenous (bolus) injection on day 0, 2 hours after T cells were implanted (h: 2) at 0.1 or 1 (5 ml/kg). Tumors and tissues (tumor, liver, spleen, and muscle) were collected 24 hours after treatment administration, immediately after euthanasia. Samples were immediately frozen in liquid nitrogen (snap-frozen samples) and kept at −80° C. until analysis.

### 6.8.2.2 Murine in Vivo Studies

For the studies involving T cell engraftment, the human T cells were obtained from fresh leukoreduction system chambers (LRS) purchased from the blood bank of the University Hospital Freiburg. The PBMCs were removed from the LRS chambers, erythrocytes were lysed with sterile ACK buffer (150 mM ammonium-chloride, 10 mM potassium bicarbonate, 0.1 mM EDTA, pH 7.2-7.4) for 3 minutes at RT, then diluted with sterile PBS containing 2 mM EDTA, and subsequently washed by centrifugation (300×g, 10 minutes, RT).

T cells were isolated from PBMCs using Miltenyi's “Pan T Cell Isolation Kit” (cat #130-096-535), stimulated with CD2-biotin, CD3-biotin and CD28-biotin antibodies with the addition of Anti-Biotin MACSiBeads (Miltenyi, cat #130-091-441) and expanded in RPMI-1640 with 10% FBS. From day 3 onwards T cells were cultivated with RPMI-1640 supplemented with 10% FBS containing IL-2 (at 20 units rIL-2 per mL). Stimulated T cells were grown for 11 days, before magnetic beads were removed on the day of engraftment into the mice. All steps were performed according to the manufacturer's instructions. On the day of randomization, T cells were counted (Nucleo Counter NC-250) and resuspended in PBS at the required concentration for injection; mice received 15×10⁶ human T cells from one of three donors IV. In addition, T cells were assessed by flow cytometry analysis for the expression of hCD45, hCD3, hCD4 hCD8, hCD69 and hPD-1 pre-engraftment on day 0 of the experiment.

The in vivo experiment using NCI-H292 cells in T cell humanized mice was set-up as following: NCI-H292 cells were grown at 37° C. in a humidified atmosphere with 5% CO₂ in RPMI 1640 medium (25 mM HEPES, with L glutamine, cat #FG1385, Biochrom) supplemented with 10% (v/v) fetal bovine serum (Sigma, cat #F9665) and 0.05 mg/ml gentamicin (Life Technologies, cat #15710064) and passaged twice weekly using TrypLE Express (ThermoFisher, cat #12605 010). Female NOG mice (NOD.Cg-PrkdcscidIl2rgtm1Sug/JicTac, Taconic) were anesthetized by inhalation of isoflurane and received 2×10⁶ tumor cells (100 μL of a suspension in PBS Matrigel) by subcutaneous injection into the right flank. Cell viability was determined before and after tumor implantation using the CASY TT Cell Counter (OLS OMNI Life Science GmbH & Co. KG, Bremen, Germany). Animals were monitored until the tumor implants reached the mean study volume criteria of 200-300 mm³, in a sufficient number of animals.

On the morning of dosing, the required number of aliquots of the test item stock solutions and vehicles were thawed at +25° C. using a water bath and swirled gently for 5-10 minutes. Stock solutions were diluted in the appropriate volume of control article under laminar flow (either commercial and sterile DPBS or Histidine/Sucrose buffer, provided ready to use, depending on the ISVD tested).

At the start of the experiments (h: 0) appropriate animals were engrafted with 15×10⁶ in vitro expanded human T cells (Texp) from three individual donors (A, B and C) via intravenous injection (IV).

### 6.8.2.3 Ex Vivo Evaluation of Protease Activated ISVD Constructs by MALDI-MSI

### Chemicals

For the ex vivo assessment of activation: compounds (T028200192 and T028200194) were prepared at a concentration of 100 μM in 20 mM Histidine pH 6.5, 8% sucrose and 0.01% Tween20. Ethanol, acetonitrile, isopropanol, chloroform, Tris, sodium chloride (NaCl) acetic acid and TFA were obtained from Thermo Fischer Scientific. Deionized water with a conductivity <20 mSi (Millipore) was prepared in-house. The MALDI matrix DHB (2,5-Dihydroxybenzoic acid) and sDHB as well as standards used for MALDI-MSI calibration or LC-MS lock mass (Insulin, Cytochrom C, Ubiquitin, Leucine enkephalin and Glu-1-Fibrinopeptide B) were obtained from Sigma Aldrich. Indium tin oxide (ITO)-coated conductive slides were obtained from Bruker Daltonics.

### Workflow

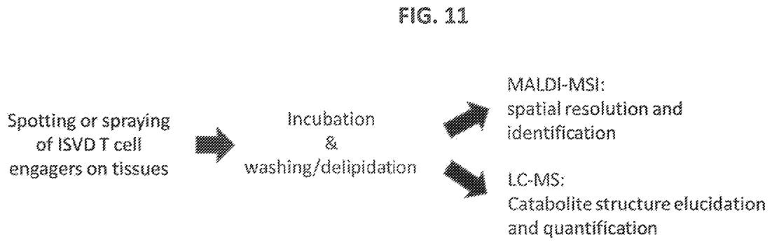

A MALDI-MSI and LC/MS workflow was established to monitor the activity and cleavage specificity of proteases in tumors and healthy tissues (FIG. 11). The workflow has also been applied to monitor in vivo distribution of protease-specific released products in tumors after treatment with T028200192 (containing a protease cleavable linker) and T028200194 (containing a non-cleavable linker).

### Sample Preparation for Monitoring the Specific Cleavage of an ISVD Construct

For MALDI-MS imaging experiments 10 μm thick tissue cryosections were cut with a cryostat (CM 1950 cryostat, Leica Biosystems), thaw-mounted onto conductive indium thin oxide coated glass slides (ITO, Bruker Daltonics) and subsequently desiccated at RT for 30 minutes until matrix application or stored at −80° C. under vacuum until further use. Dilution series of ISVD solutions from 0.01 to 100 pmol in Tris buffer (50 mM Tris, 100 mM NaCl) were freshly prepared and pipetted onto tissues sections or ITO glass and dried for 30 minutes in vacuum.

Spotted tissue sections were then incubated at 37° C. and 95% humidity in a CO₂ incubator BBD6220 chamber (Thermo Scientific) or using the SunDigest device (SunChrom) for 1, 2, 6 and 24 h in time-course experiments and for 24 h in activation/specific cleavage assessment. The lipid background was removed by a six-step washing protocol as previously described (Yang and Caprioli 2011): Briefly, slides were immersed in 70% ethanol for 30 s; 100% ethanol for 30 s, Carnoy's fluid (60:30:10 ethanol/chloroform/acid acetic) for 2 min; 100% ethanol for 30s; ddH₂O for 30 s and finally 100% ethanol for 30 s. Excluding the solution for the last two steps, all washing solution were stored at −20° C. for about 1h before use. The last two solutions were cooled on ice. After lipid washing, glass slides were dried at least 2h or overnight in vacuum.

### Sample Preparation for Monitoring the Spatial Distribution of Protease Activated ISVD Constructs

To monitor the activation of the ISVD construct on smaller tissues (skin, intestine, or spleen) or to visualize spatial distribution of protease activated ISVD product in tumor, homogeneous layers of ISVD construct molecules (20 pmol/μL) solutions were applied on tissue sections using the SunCollect spray system (SunChrom) and the following spray protocol were applied: 20 layers with spray-head velocity of 300 mm/min at a height of 3 cm, 22 mm line distance 2 mm and a flow rate of 15 μL/min. The final amount of ISVD construct on tissue was 10 pmol/mm². After the spraying, the slide was dried in desiccator for 3 min, then incubated and washed as described above for the spotted tissues.

### Matrix Deposition and MALDI Mass Spectrometry Imaging

Tissue slides were spray-coated with sDHB matrix (60 mg/ml in 40:60 ACN/ddH₂O supplemented with 0.5% TFA) using SunCollect sprayer (SunChrom) according to a reported protocol (Munteanu, Meyer et al. 2014, Fülöp, Sammour et al. 2016): 5 layers with a speed of 300 mm/min and 2 mm line distance, flow rate=10 μL/min for the first two layers and 15 μL/min for all other layers; a drying step of 3 min including a short drying on a heating plate (50° C.) was introduced between each cycling step.

Tissue sections were then analyzed using a rapiflex MALDI-TOF mass spectrometer (Bruker Daltonics) in positive ion-linear mode in a mass range of 4-20 kDa. Tissues were measured at a lateral resolution of 100 μm (50 μm for the monitoring of spatial distribution), summing up 2500 shot/pixel. The matrix suppression cut off mass was set to 3 kDa, PIE delay to 250 ns and laser repletion rate to 10000 Hz. External quadratic calibration was performed using a mixture of insulin (0.5 μmol), cytochrome C (10 μmol) and myoglobin (10 μmol). Data was acquired and processed using flexControl V4.2, Fleximaging V6.0 and using SCiLS Lab MVS (V9.01.12514, Bruker Daltonics).

### Data Visualization and Statistical Analysis

Regions of interest (ROIs) were drawn on tissues around the ISVD spots to export mean spectra from SCiLS Lab into mMass V5.5.0 for baseline-subtraction and peak picking (S/N>5). Intensities of the m/z-values of interest were exported into GraphPad Prism 9 or TIBCO Spotfire Analyst V10.3.3 software for data visualization and statistical analysis. Protease cleavages products were identified using Sequence Editor V3.2 (Bruker Daltonics) with a mass tolerance of 5 Da.

### 6.8.2.4 Catabolite Structure Elucidation of the ISVD Constructs by LC-MS

### Sample Preparation

Fresh frozen tissue sections (10 μm) were mounted on ITO glass slide, spotted with ISVD solution (100 pmol in Tris buffer) and incubated for 24 h as described above for MALDI-MSI measurement.

The tissues were then removed from the slides by scraping carefully with a scalpel and transferred into a tube. To homogenize the tissue and extract ISVD and potential cleavage products, 0.1% formic acid in water (10 μl/tissue section) was added. After sonication on ice and centrifugation at 4° C., 8000 rpm for 10 min, 10 μL of the supernatant were injected into the 2D-LC/MS system.

### 2D-LC/MS Method

An ACQUITY UPLC I-Class System with 2D technology (Waters Company, MA, USA) configured in the heart-cutting mode was used for two-dimensional chromatography. The first separation was performed by size exclusion chromatography (SEC) on a Waters Protein BEH SEC column (1.7 μm, 150×2.1 mm), kept at 25° C., and operated at 140 μL/min. The SEC separation was carried out in isocratic mode using DPBS as mobile phase and recorded by UV at 280 nm, 230 nm and 214 nm. The analytes of interest, eluting between 7 and 12 min in the first chromatographic dimension were transferred to a trap column using a switching valve. The trapping used for sample concentration and to achieve mobile phase compatibility for fraction transfer to the second dimension, was performed on a Waters BEH C4 column (1.7 μm, 50×2.1 mm), maintained at 80° C. and operated at 700 μL/min. Eluent A consisted of 0.1% formic acid in water and eluent B of 0.1% formic acid in acetonitrile. The following gradient was used: 3% B was hold for 9 min followed by a drastic increase to 15% B in 0.1 min to transfer concentrated analytes to the second dimension. The analytes were then separated on the second dimension using the same stationary phase as the trap column (Waters BEH C4 column; 1.7 μm, 50×2.1 mm). Mobile phases contained 0.1% formic acid in water (Eluent C) and 0.1% formic acid in acetonitrile/isopropanol (1:1; v/v) (Eluent D). A binary gradient at a flow rate of 300 μL/min and a column oven temperature of 80° C. was applied as follows: 5% D was hold for 4 min followed by two steps of linear increases to 30% D in 21 min and 95% D in 2 min. The total run time for the entire 2D-LC method was 60 min.

Mass spectrometry (MS) experiments were performed on a QTOF instrument (Synapt G2-Si, Waters Company, MA, USA), in positive ion electrospray mode. The operating parameters were as follows: capillary voltage=3.0 kV, sampling cone=120 V, source offset, 80 V, nebulization gas pressure=6.5 bar, source temperature, 150° C., desolvation temperature, 500° C. MS spectra were acquired in the time window 16-45 min, over the m/z 400 to 4000 mass range, in sensitive mode and with a scan time of 1s. To improve mass accuracy, a reference solution containing Leucine enkephalin (100 fmol/μL) and Glu-1-Fibrinopeptide B (255 fmol/μL) was constantly infused during measurement using LockSpray™. Data were processed using Genedata Expressionist® V14.0 and ISVD cleavage products were identified using GPMAW V13.02 (General Protein/Mass Analysis; Lighthouse data).

### Data Evaluation and Structure Elucidation

The raw data were directly imported into the Expressionist software package (version 16.0.2, Genedata). An adapted and application-specific workflow was built and used for retention time and m/z restriction (RT 16-45 min; m/z 600-4000), chemical noise reduction, spectrum smoothing, time-resolved deconvolution as well as peak detection. Briefly, all MS chromatograms were smoothed with RT Window=3 scans. To remove the background noise, a peak intensity is defined as follows:

$${Intensity}_{subtracted} = max ( {Intensity}_{original} - Quantile - Threshold , 0 )$$

Here, values Quantile=50% and Intensity Threshold=10 cps were used. Furthermore, signals satisfying at least one of the following criteria were considered as noise peaks and subtracted: RT Window>51 scans, Minimum RT Length=4 scans, or Minimum m/z Length=7 data points. The scan-per-scan deconvolution was performed with the embedded Genedata algorithm with typical parameters: Maximum entropy deconvolution method with 1.0 Da step.

The identification of the deconvoluted peaks is then carried out using the GPMAW software (version 13.02, Lighthouse data). It enables the search for possible structures from the sequence of the intact ISVD and from the mass list of the protease-released products detected in the deconvoluted spectra. The mass tolerance was set at 100 ppm and the following modifications were considered: dehydration (—H₂O₁), hydration (H₂O₁), methylation (C₁H₂), oxidation (O₁), amidation (H₂N₁—O₁H₁), deamidation (—H₂N₁+O₁H₁), di-dehydro (—H₂), dehydro (—H₁), succimide (—H₃N₁ or —H₂O₁).

### 6.8.2.5 In Vivo Assessment of Protease Mediated Activation of ISVD Constructs

### Cryosectioning and Preparation of In-Vivo Samples for MALDI-MSI and LC-MS Analyses

The in vivo samples (liver, spleen, muscle, and tumor) collected after 24 h dosing was sectioned (10 μm) and the central tissue sections were measured as described previously for the ex-vivo experiments: 1) by MALDI-MSI to monitor the spatial distribution of the ISVD and its protease cleavage products, 2) by LC-MS to confirm the identification and monitor the intact ISVD.

### 6.8.2.6 Results of the Ex Vivo and In Vivo Assessment of Protease Cleavage of ISVD Constructs

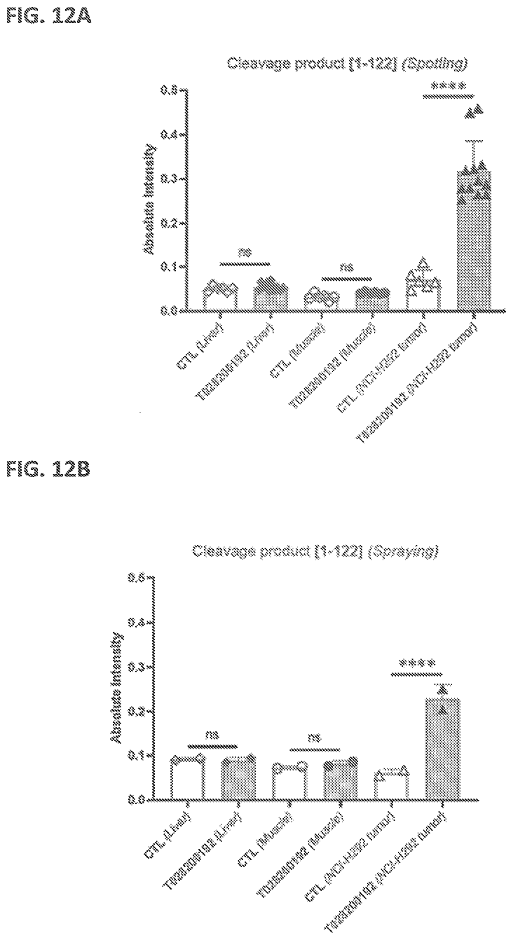

Ex vivo evaluation of the activation of the EGFR ISVD construct containing a protease cleavable linker (T028200192) was done by spotting the compound on the frozen tissue sections or by applying the compound via a homogenous spray. Spotting the compound on liver, muscle and tumor tissue showed a significantly increased intensity for construct T028200192 cleaved after amino acid 122 (corresponding to matriptase and uPA cleavage site in the protease cleavable linker) compared to the control (CTL, buffer) (FIG. 12A). Using the same methodology, no significant differences in absolute intensity were observed in liver and muscle between buffer (CTL) and spotting of compound T0282000192.

Spraying the compound on the tissue sections confirms the identification of fragment 1-122 (corresponding to the uPA and matriptase cleavage site in the cleavable linker) on tumor tissue while no significant detection of the fragment on liver and muscle sections (FIG. 12B).

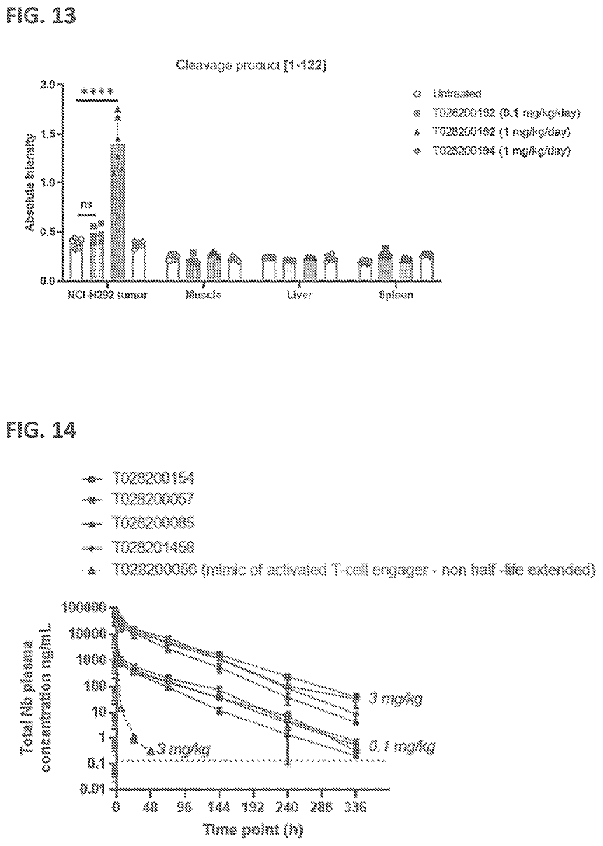

Analysis of the tissues derived from the treated NCI-H292 xenograft tumor model (treated with compound T028200192 or with compound T028200194) is shown in FIG. 13. The data clearly shows that the fragment 1-122 (corresponding to the fragment cleaved by uPA or matriptase) can be detected in the tumor from the mice treated with the 1 mg/kg dose while no significant fraction of the same cleavage product (1-122) can be detected in muscle, liver and spleen of the same animals (FIG. 13). Additionally, no fragments could be detected from the same construct but with a non-cleavable linker instead of the construct with the protease cleavable linker (T028200194 vs. T028200194, resp.).

In the ex vivo evaluation of cleavage as well as in the in vivo evaluation of cleavage the correct mass of the cleaved product could be confirmed using LC-MS.

### 6.9 Example 9: Mouse Plasma PK of a Precision Activated ISVD Construct

In this example, the following (HER2) ISVD constructs were tested: T028200154, T028201458, T028200085, T028200056, and T028200057.

### Protocol

The PK assessment was performed in CD1 mice (Outbred, SPF-Quality, Charles River Deutschland, Sulzfeld, Germany), between 7 to 9 weeks old at initiation of dosing. The CD1 mouse was chosen as the animal model as it is an accepted rodent species for preclinical toxicity testing by regulatory agencies, and this strain is the most representative of the in vivo preclinical pharmacological models available.

On the morning of dosing, the required number of aliquots of the test item stock solutions and vehicles were thawed at +25° C. using a water bath and swirled gently for 5-10 minutes. Stock solutions were diluted in the appropriate volume of control article under laminar flow: commercial and sterile D-PBS for T028200154, T028201458, T028200085, T028200056, and Histidine/Sucrose buffer (20 mM Histidine/8% Sucrose/0.01% Tween20) for T028200057.

The test items were administered to the animals via a single intravenous (bolus) injection to the tail vein, without sedation. Blood was collected at different timepoints (0.083 h, 0.5 h, 1 h, 6 h, 24 h, 72 h and 144 h for T028200154, T028201458, T028200085, T028200056; and 0.083 h, 0.5 h, 1 h, 6 h for T028200057) via the jugular vein. Blood was sampled with a composite profile, with alternating blood collection from the 1^{st}-3^{rd} and the 4^{th}-6^{th} animals of each group (n=6). The animals were restrained during blood sampling for intermediate sampling. Blood (0.1 mL per sample) from the jugular vein was collected using a 25G needle in labelled 0.5 mL micro tubes containing 3.8% citrate (1:9 ratio for citrate:blood). Immediately following collection, blood samples were slowly homogenized and stored at room temperature. For terminal sampling, blood was collected via aortic puncture following isoflurane inhalation anesthesia. Blood samples were collected into labelled 1.5 mL micro tubes containing 3.8% citrate (1:9 ratio for citrate:blood). Immediately following collection, blood samples were slowly homogenized and kept at room temperature. Whole blood was processed to plasma by centrifugation, within 30 min of collection, at 1500 g for 10 minutes at 2-8° C. After centrifugation the resulting plasma was collected, placed on wet ice and stored in labeled polypropylene tubes (Micronic) and stored in an ultra-low freezer to maintain −80° C. until shipment on dry ice.

### Measurement of ISVD Concentrations in Plasma

The Total PK assay detects the protease activated (if present) and masked (i.e., intact) fractions of the different masked constructs.

A streptavidin-coated MSD GOLD 96-well SMALLSPOT® plate (Meso Scale Discovery, cat #L45SA) was blocked with Superblock T20 (Thermo Scientific, cat #37536) for 30 minutes at RT. The plate was then washed with PBS/0.05% Tween20 and incubated for 1 hour at RT and at 600 rpm with 0.25 ug/mL biotinylated RMAT003, a polyclonal antibody (pAb) specifically directed against the TCR binding moiety of the different ISVDs. Calibrators and Quality Controls (QCs) were prepared in pooled mouse plasma. After washing, calibrators, QCs and study samples were applied at a Minimum required dilution (MRD) of 10 in PBS/0.1% casein/0.05% Tween20 and incubated for 1 hour at RT and at 600 rpm.

After washing, for the total PK assay, the plate was incubated for 1 hour at RT and at 600 rpm with 0.5 μg/mL sulfo-labelled ABH0071, a monoclonal antibody (mAb) directed against the framework of the different ISVD building blocks.

After washing, MSD Read buffer A (Meso Scale Discovery) was added and ECL values were measured with a Sector Imager Quickplex SQ 120 (Meso scale Discovery).

Calibration curve responses from the total assay were processed using a cubic-1/Y² weighted fit of electrochemiluminescence (ECL) responses versus concentrations. Calibration curve responses from the intact assay were processed using a 5PL-1/Y² weighted fit of ECL responses versus concentrations. The concentrations of calibrators, QC and study samples (reported values) were calculated by interpolation based on the fit of the calibration curve.

### Results

HER2 ISVD constructs (T028200057, T028200085, T028201458) with a protease cleavable linker have similar plasma PK profiles compared to a control ISVD construct (T028200154) with a non-protease cleavable linker. Construct T02820056, which mimics a protease pre-activated ISVD construct (i.e., without half-life extension), show a fast clearance (FIG. 14). These data demonstrate that HER2 ISVD constructs (T028200057, T028200085, T028201458) with a protease cleavable linker have an improved PK profile compared to non-half-life extended HER2 ISVD constructs: the albumin masked and inactive pro-drug is highly stable in mouse blood in vivo and the active form of the drug (T028200056—non half-life extended) is quickly cleared from blood circulation.

### 6.10 Example 10: ISVD Construct with a Protease Cleavable Linker is Stable in Plasma of Tumor Bearing Mice

In this Example, the following HER2 ISVD constructs were tested: T028200333, T028200324, and T028200323. The T028200333 and T028200324 ISVDs present a GCGG sequence tag at the N-terminus and the T028200323 ISVD presents a GGC sequence tag at the C-terminus.

### Protocol

### Radiolabeling Procedure

This procedure was adapted from a validated two-step procedure (Vosjan M J, Perk L R, Visser G W, Budde M, Jurek P, Kiefer G E et al. Conjugation and radiolabeling of monoclonal antibodies with zirconium-89 for PET imaging using the bifunctional chelate p-isothiocyanatobenzyl-desferrioxamine. Nat Protoc 2010; 5:739-743). To avoid protein complexation, ISVDs were mixed overnight at 4° C. with Dithiothreitol (DTT) reducing agent at a final concentration of 10 mM. Then, the protein was purified using a spin column with D-PBS buffer. A cysteine site specific conjugation with a maleimide-DFO was performed and followed by radiolabeling with a Zr89 isotope (Perkin Elmer). Deferoxamide-maleimide (Macrocyclics) was dissolved in dimethyl sulfoxide at a concentration of 10 mM and added at a 10-M excess to the protein solution. After stirring overnight at room temperature under 550 revolutions/min stirring, the reaction mixture was purified with a spin column in D-PBS buffer. An ⁸⁹Zr-oxalate, ^{˜}1 mCi, solution in 1 M oxalic acid solution (56 μL) was neutralized with 2 M Na2CO₃ (25 μL). After 3 min, 0.10 ml of 0.5 M HEPES (pH 7.1-7.3), desferrioxamine-ISVD solution (pH 7.4), and 0.10 mL of 0.5-M HEPES buffer were successively added into the reaction vial. After incubation for 60 min at room temperature under low stirring, the ⁸⁹Zr labelled-ISVD construct was purified with a spin column in D-PBS buffer.

### Blood Pharmacokinetics and Tumor Specific Uptake of ⁸⁹Zr-HER2 T-Cell Engagers in HCC1954 Tumor Model

HER2 positive HCC1954 cells (5×10⁶) were implanted subcutaneously (0.2 mL, 50% D-PBS, 50% Matrigel) on the flank of NSG mice at day 0. On day 14, when the tumor size ranges between 100-150 mm³, the mice were treated with ⁸⁹Zr T028200333, T028200324 or T028200323 ISVDs (0.5 mg/kg) by intravenous bolus (5 ml/kg) injection through the tail vein (N=3 per group). Circulating ⁸⁹ZR-protein was assessed by gamma counting measurements (Wizard, PerkinElmer, Waltham, MA, USA) in blood samples (5 μL) at 0.083, 1, 2, 4, 6, 24, 48, 72, and 144 hours post-injection. PET/CT images were acquired (Inveon, Siemens) for in vivo biodistribution at 4, 24, 48, 72, and 144 hours post-injection. At the last time point, mice were sacrificed for necropsy. Tissue biodistribution of ⁸⁹ZR-protein was assessed by gamma counting in lung, heart, liver, kidney, spleen, muscle, blood, urine, and tumor.

⁸⁹ZR-protein concentration was expressed in nanogram equivalent per milligram of tissue, adjusted for specific radioactivity and [⁸⁹Zr] decay corrections.

### Results

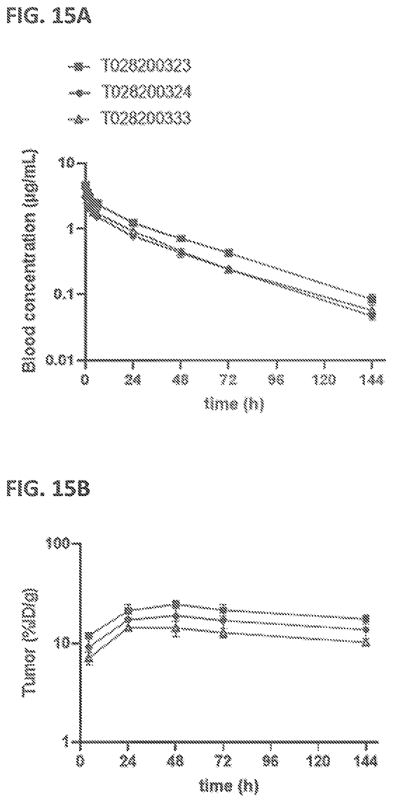

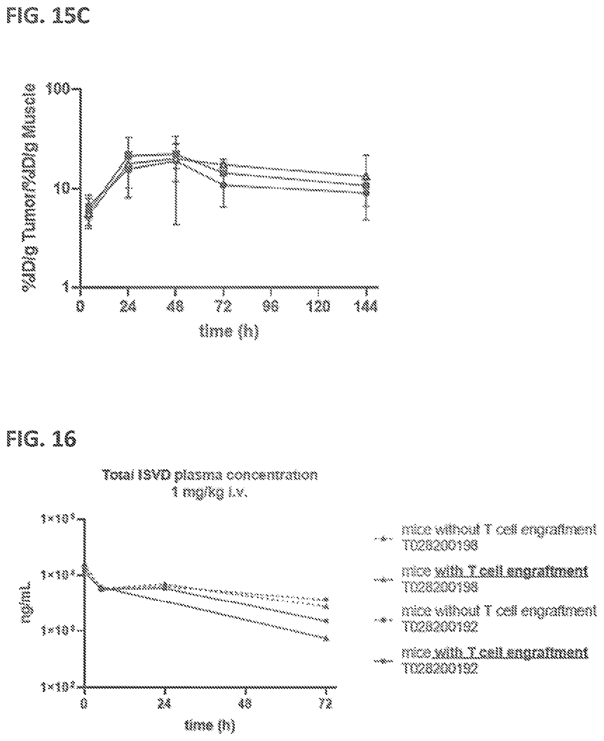

T028200323 with a protease cleavable linker is stable in mouse blood of tumor bearing mice, as was demonstrated already in healthy CD1 mice (see Example 9, T028200057). After normalization, the total concentrations of the ISVD constructs (intact and cleaved) in the HCC1954 tumors (i.e., HER2 positive) are similar for the different ISVD constructs (FIG. 15A). In tumor, the concentrations of the different ISVD constructs are similar, leading to the conclusion that the 3 ISVD constructs have similar tumor targeting capabilities (FIG. 15B and FIG. 15C).

### 6.11 Example 11: ISVD Constructs with Protease Cleavable Linker Show an Improved Plasma PK Profile in Tumor Bearing Mice, Engrafted with Human T Cells

In this Example, the following EGFR ISVD constructs were tested: T028200198 and T028200192.

### Materials and Methods

### PBMC Preparation and T Cell Expansion for T Cell Humanized Mouse Model

The protocol used for PBMC preparation and T cell expansion is as described in Example 8.

### In Vivo Study Protocol of NCI-H292 Xenograft Mouse Model

The study protocol is the same as is described in Example 8.

Blood was collected at different timepoints after dosing via the retrobulbar route, under isoflurane anesthesia. Plasma was prepared by collecting the blood in standard plasma vials coated with sodium citrate as anticoagulant (Sarstedt, cat #41.1506.005 citrate sample volume 1.3 mL) at room temperature directly followed by centrifugation at 9600×g for 2 min at RT. Plasma was transferred to a new tube, and samples were stored at −80° C. prior to shipment. PK analysis was performed on plasma samples with the total PK assay.

### Administration of Therapy

All treatments were administered to the appropriate animals via a single intravenous (bolus) injection on day 0, 2 hours after T cells were implanted (h: 2) at 1 mg/kg (5 ml/kg).

### Measurement of ISVD Concentrations in Plasma

ISVD concentrations were determined as described in Example 9.

### Results

T028200192 with a protease cleavable linker has an improved plasma PK profile in tumor bearing NOG mice engrafted with human T cells, compared to the active construct T028200198 (FIG. 16). T028200198 shows a faster clearance in plasma for mice engrafted with human T cells, compared to non-engrafted mice (target mediated clearance) (FIG. 16). These data demonstrate that the masked ISVD construct T028200192 (EGFR) binds less circulating T cells in mouse blood.

### 6.12 Example 12: ISVD Constructs with a Protease Cleavable Linker Show Tumor Specific Activation In Vivo

In this Example, the following EGFR ISVD constructs were tested: T028200194 and T028200192.

### Protocols

### PBMC Preparation and T Cell Expansion for T Cell Humanized Mouse Model

The protocol used for PBMC preparation and T cell expansion is as described in Example 8.

### In Vivo Study Protocol in NCI-H292 Xenograft T Cell Humanized Mice

The study protocol is the same as was described in Example 8.

### Administration of Therapy

All treatments were administered to the appropriate animals via a single intravenous (bolus) injection on day 0, 2 hours after T cells were implanted (h: 2) at 0.1, 1 and 10 mg/kg (5 ml/kg).

### Preparation of Tumor and Spleen Lysates

For PK analysis, tumors and spleens were collected 24 h after treatment administration, immediately after euthanasia. Samples were immediately frozen in liquid nitrogen (snap-frozen samples) and kept at −80° C. until lysate preparation.

Tumor and spleen samples were lysed on the day of the PK analysis. Tumor samples were thawed at RT and crushed with a glass tissue grinder in a lysis buffer (1× RiPa buffer Millipore, cat #20-188, mixed with 1× protease inhibitor cocktail Roche, cat #11 836 145 001). The volume of lysis buffer was adjusted to the tumor weight (1 μL 1×Ripa/1 mg tumor). Next, the lysed tumor samples were incubated on ice for 30 minutes. Then, the samples were centrifuged for 10 minutes at 16100 g. Supernatant was taken and used immediately in the assay; the leftover supernatant was stored at −80° C.

### Measurement of ISVD Concentrations in Tissue Lysates

Two different assays were set up to measure the ISVD concentrations in tissue lysates: a Total PK assay and an Intact PK assay. The Total PK assay detects the cleaved and uncleaved masked fractions of the different masked constructs. The Intact PK assay detects only uncleaved masked (i.e., masked) constructs.

In both assays, a streptavidin-coated MSD GOLD 96-well SMALLSPOT® plate (Meso Scale Discovery) was blocked with Superblock T20TM (Thermo Scientific) for 30 minutes at RT. The plate was then washed with PBS/0.05% Tween20 and incubated for 1 hour at RT and at 600 rpm with 0.25 μg/mL biotinylated RMAT003, a pAb specifically directed against the anti-TCR ISVD.

Calibrators and Quality Controls (QCs) were prepared in pooled mouse plasma. After washing, calibrators, QCs and study samples were applied at a Minimum required dilution (MRD) of 10 in PBS/0.1% casein/0.05% Tween20 and incubated for 1 hour at RT and at 600 rpm.

After washing, for the Total PK assay, the plate was incubated for 1 hour at RT and at 600 rpm with 0.5 ug/mL sulfo-labelled ABH0071, a mAb directed against the framework of the different ISVD building blocks.

For the Intact PK assay, after washing, the plate was incubated for 1 hour at RT and at 600 rpm with 0.2 pg/mL sulfo-labelled ABH0085, a mAb specifically directed against the anti-albumin (ALB) ISVD.

After washing, MSD Read buffer A (Meso Scale Discovery) was added and ECL values were measured with a Sector Imager Quickplex SQ 120 (Meso scale Discovery).

Calibration curve responses from the total assay were processed using a cubic−1/Y² weighted fit of electrochemiluminescence (ECL) responses versus concentrations. Calibration curve responses from the intact assay were processed using a 5PL−1/Y² weighted fit of ECL responses versus concentrations. The concentrations of calibrators, QC and study samples (reported values) were calculated by interpolation based on the fit of the calibration curve.

### Results

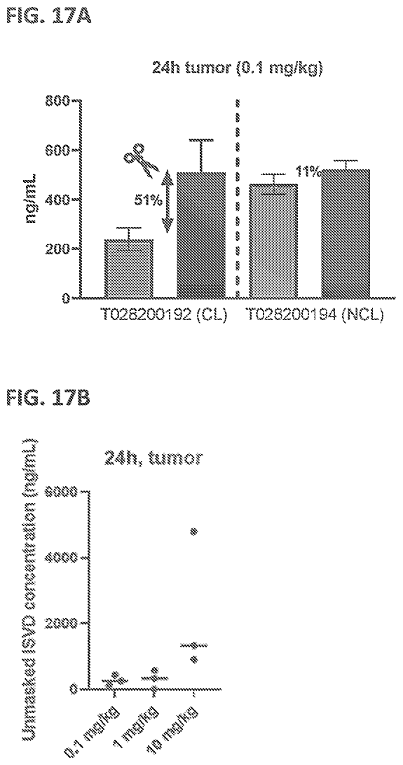

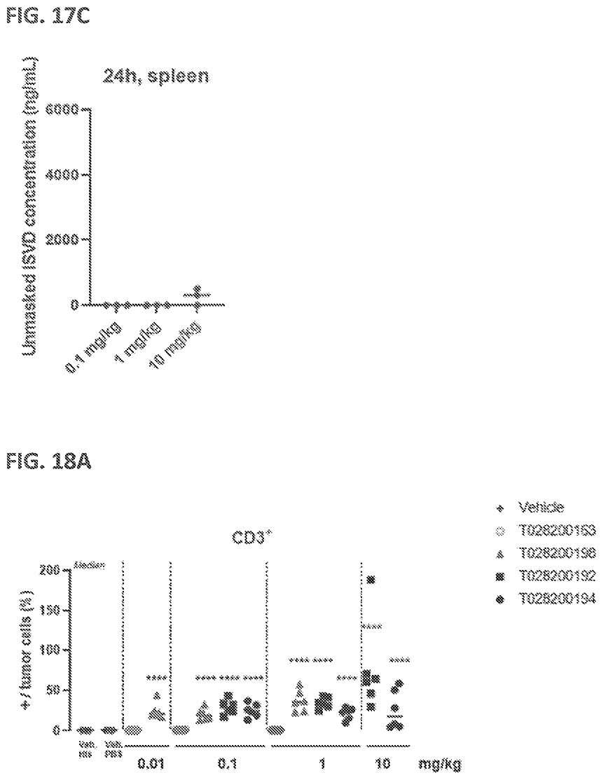

T028200192 shows specific cleavage in the tumor tissue collected from tumor bearing NOG mice engrafted with human T cells (FIG. 17A). The concentration of cleaved and unmasked product from T028200192 increases with dose level (from 0.1 to 10 mg/kg, single dose) at 24 h in the tumor (FIG. 17B) but not in the spleen, used as control tissue (FIG. 17C). The mean % of cleavage of ISVD construct is 51% for T028200192 at 0.1 mg/kg, compared to 11% for T028200194 with non-protease cleavable linker (FIG. 17A). These data demonstrate unmasking of a precision activated ISVD construct in vivo in the tumor microenvironment. These data are well correlated with the ex vivo data shown in Example 8.

### 6.13 Example 13: Tumor T Cell Infiltration of an ISVD Construct with a Protease Cleavable Linker

In this Example, the following EGFR ISVD constructs were tested: T028200163, T028200198, T028200194, and T028200192.

### Protocols

### PBMC Preparation and T Cell Expansion for T Cell Humanized Mouse Model

The protocol used for PBMC preparation and T cell expansion is as described in Example 8.

### In Vivo Study Protocol of NCI-H292 Xenograft T Cell Humanized Mice

The study protocol is the same as is described in Example 8.

### Administration of Therapy

All treatments (vehicles and ISVD constructs) were administered to the appropriate animals via a single intravenous (bolus) injection on day 0, 2 hours after T cells were implanted (h: 2) at 0.01, 0.1, 1 and/or 10 mg/kg (5 mL/kg).

### Preparation of Formalin-Fixed Paraffin-Embedded (FFPE) Tumor Sample

For PD analysis, tumors were collected 72h after treatment administration, immediately after euthanasia. Tumors were fixed in 10% neutral buffered formalin for approximately 24 hours. The fixative was then replaced by submerging the samples in 70% ethanol for up to seven days. Thereafter, samples were dehydrated by sequentially incubating them in the following solutions: 70% ethanol (two times 0.5 h), 80% ethanol (two times 1 h), 100% ethanol (two times 0.5 h), 100% isopropanol (1.5 h), xylene (two times: 1 h; 1.5 h). Finally, samples were infiltrated by, and embedded in, paraffin. Sections (5 μm in thickness) were prepared from the paraffin block and mounted on microscopy cover slides (one section per slide).

Paraffin was removed from the FFPE tissue sections with clearing solvent (CytoVista™ Tissue Clearing Reagent, cat #V11300 ThermoFisher) (3×4 min) followed by rehydration of the tissue with subsequent incubations of 100% EtOH (2×2 min), 70% EtOH (1×2 min), MilliQ water (1×2 min) and PBS (1×5 min). Antigen retrieval was performed by incubation in 10 mM Tris/1 mM EDTA/0.05% Tween20 for 20 min in the pressure cooker. After the slides were cooled down, they were washed with MilliQ water for 2 min followed by PBS/0.05% Tween20 for 3×5 min. Endogenous peroxidase in the tissue was inactivated with dual endogenous enzyme block (DAKO) for 10 min at RT. After washing with PBS/0.05% Tween20, the tissues were blocked with 10% goat serum (Sigma-Alrich, cat #G9023) for 60 min at RT. After a wash with PBS/0.05% Tween20, the tissue sections were incubated ON at 4° C. with either 0.195 ug/mL rabbit anti-human CD45 (Abcam, cat #ab40763), 0.07 μg/mL rabbit anti-human CD4 (Abcam, cat #ab133616), 0.25 μg/ml rabbit anti-human CD3 (Abcam, cat #ab52959) or 0.1 μg/mL rabbit anti-human CD8a antibody (Sigma, cat #HPA037756). The following day, after washing with PBS/0.05% Tween20, the sections were incubated with an HRP-labeled anti rabbit polyclonal antibody (DAKO, cat #K4003) for 30 min at RT. After washing, the sections were stained with DAB Enhancing Solution (VectorLabs, cat #H-2200) for 6 min at RT. The chromogenic reaction was stopped by washing 3×5 min with MilliQ water and nuclei were stained with hematoxylin (Sigma-Alrich, cat #H3136) counterstain for 2 min at RT. After washing with MilliQ water, the tissue sections were dehydrated by subsequent incubations in MilliQ water (1×2 min), 70% EtOH (1×2 min), 100% EtOH (2×2 min) and clearing solvent (3×4 min). Finally, the slides were mounted using non-aqueous mounting medium (VectorLabs).

The number of positive T cells and tumor cells in the stained sections was calculated using the Qupath software (version 0.2.2; open-source software). First, the watershed cell detection protocol for DAB/hematoxylin staining was applied using a background correction of 40. Next, the software was trained in distinguishing positive T cells and tumor cells using classifiers. Both protocols were used to write the final method script that was used for all slides. An exporting script was used to retrieve the data.

### Statistical Analysis

For statistical analysis purpose, the percentage of T cell population compared to the tumor cells were compared between each treatment group and its respective vehicle, for each T cell marker. Parameter values have been log-transformed to relieve variance heterogeneity. A mixed effects model including T cell marker, treatment and dose effect and their interaction was performed, followed by a contrast analysis with Bonferroni-Holm adjustment for multiplicity. The statistical analyses were performed using SAS 9.4 for Windows 10. Values of P<0.05 were considered significant.

### Results

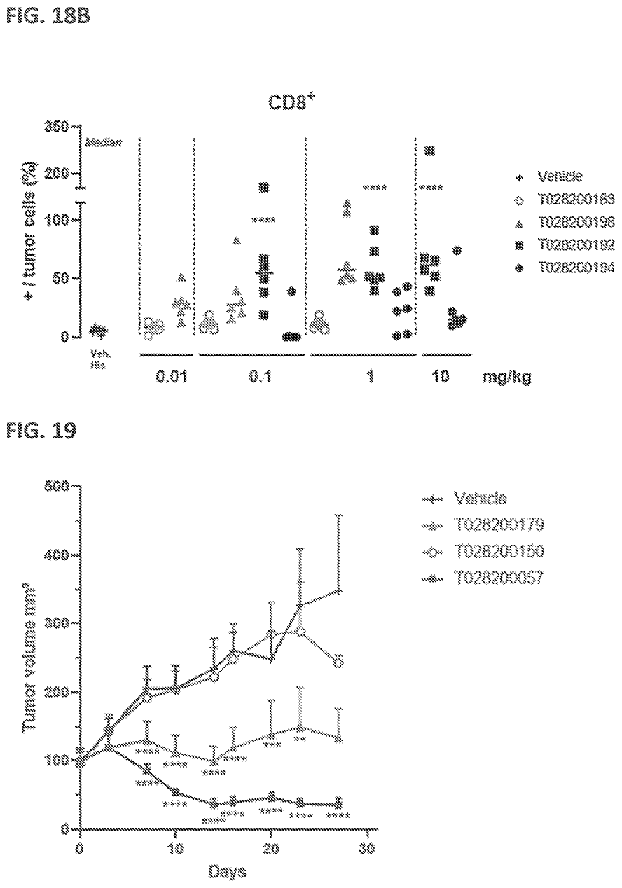

As illustrated in FIG. 18A, treatment with protease activated T028200192 resulted in CD3⁺ T cell infiltration into the tumor at 72 h, at the same level as the T028200198 administrated to the mice at the same dose levels (0.1 and 1 mg/kg). In clinical studies, a higher number of tumor infiltrating lymphocytes, and in particular CD8⁺ cells, is associated with better clinical outcomes and they are considered important biomarkers for the prediction responses to cancer therapy (Mahmoud, S. M. et al. Tumor-infiltrating CD8⁺ lymphocytes predict clinical outcome in breast cancer. J. Clin. Oncol. 29, 1949-1955 (2011)). The fully masked ISVD construct T028200194, containing a non-protease cleavable linker, is less potent in attracting CD8⁺ T cells to the tumor, compared to the construct with a protease cleavable linker T028200192, as shown in FIG. 18B. T cells are not detected in the tumors from mice treated with vehicles or a construct without the T cell engaging ISVD (T028200163) (FIG. 18).

These data demonstrate an efficient and specific activation of human T cells by precision activated ISVD construct in vivo in the tumor micro-environment. These data are aligned with the data of example 12 showing a specific cleavage in the tumor.

### 6.14 Example 14: A HER2 ISVD Construct with a Protease Cleavable Linker Shows a Significant Anti-Tumoral Growth Effect

In this Example, the following HER2 ISVD constructs were tested: T028200179, T028200150, and T028200057.

### Protocols

### PBMC Preparation and T Cell Expansion for T Cell Humanized Mouse Model

Human T cells were obtained from fresh leukoreduction system chambers (LRS) purchased from the blood bank of the University Hospital Freiburg. Cells were removed from the LRS chambers into 50-mL Falcon tubes, the LRS chambers were rinsed twice with 1 mL PBMC buffer (2 mM EDTA in PBS) which was added to the cells in Falcon tubes. Cells were pelleted by centrifugation at 300×g for 5 min at RT. Erythrocytes were lysed by resuspension of the cell pellet in 20 ml sterile ACK buffer (150 mM ammonium-chloride, 10 mM potassium bicarbonate, 0.1 mM EDTA, pH 7.2-7.4), incubation for 1-3 min and centrifugation at 300×g for 5 min at RT. After removal of the supernatant, cells were resuspended in 40 mL PBMC buffer and were washed by centrifugation (200×g, 10 min RT). Cells were resuspended in 40 mL PBMC buffer and were counted. Cells were transferred to a new tube and CD3+ T cells were isolated using the Miltenyi Biotec “Pan T Cell Isolation Kit” (cat #130-096-535) according to the manufacturer's instructions. Cells were pelleted by centrifugation at 300×g for 5 min at RT and were resuspended in in X-VIVO™ 15 medium (Lonza, cat #Be02-060F containing L-glutamine, gentamicin, and phenol red). CD3+ T cells were expanded by plating 5×10⁶ cells in each well of G-Rex® 6M well culture plates (Wilson Wolf, cat #80660M) in X-VIVO™ 15 medium containing IL-2 at 10 ng/ml (Miltenyi, cat #130-097-743). The anti-Biotin MACSiBead Particles (beads) loaded with biotinylated CD2, CD3, and CD28 antibodies from the T cell Activation/Expansion Kit, human (Miltenyi Biotec, cat #130-091-441) were prepared according to the manufacturer's instructions and the bead solution was added to the T cells in G-Rex plates at a bead to cell ratio of 1:2. The T cells were cultivated in this medium (40 mL), with 40 mL fresh medium added on Day 4. On Day 7, medium was gently removed, and 40 mL fresh medium was added to the cells. Cells were harvested on Day 8 of the culture. The magnetic beads were removed directly prior to engraftment into mice using the MACSiMAG separator (Miltenyi, cat #130-092-168) according to the manufacturer's instructions. Cells were resuspended in serum-free RPMI medium and counted (Nucleo Counter NC-250) before resuspension in an appropriate amount of PBS for engraftment into mice. T cells were evaluated by FC analysis on the same day.

On the day of randomization, T cells were counted (Nucleo Counter NC-250) and resuspended in PBS at the required concentration for injection; mice received 15×10⁶ human T cells from one of three donors IV. In addition, T cells were assessed by FC analysis for the expression of hCD45, hCD3, hCD4 hCD8, hCD69 and hPD-1 pre-engraftment on Day 0 of the experiment.

### In Vivo Study Protocol of HCC1954 Xenograft T Cell Humanized Mouse Model

HCC1954 were grown at 37° C. in a humidified atmosphere with 5% CO₂ in RPMI 1640 medium (anprotec cat #AC-LM-0054) supplemented with 10% (v/v) fetal bovine serum (Sigma cat #F9665) and 0.05 mg/ml gentamicin (Life Technologies, Karlsruhe, Germany) and passaged using TrypLE Express (Thermo Fisher, cat #12605 010). Female NOG mice (NOD.Cg-PrkdcscidIl2rgtm1Sug/JicTac, Taconic, Denmark) animals were anesthetized by inhalation of isoflurane and received 1×10⁷ tumor cells (200 μL of a suspension in PBS with 50% Matrigel) by s.c. injection into the right flank. Cell viability was determined before and after tumor implantation using the CASY TT Cell Counter (OMNI Life Science GmbH & Co. KG, Bremen, Germany). Animals were monitored until the tumor implants reached the mean study volume criteria of 100-150 mm³, in a sufficient number of animals.

On the morning of dosing, the required number of aliquots of the test item stock solutions and vehicle were thawed at +25° C. using a water bath and swirled gently for 5-10 minutes. Stock solutions were diluted in the appropriate volume of control article under laminar flow (either commercial and sterile DPBS or Histidine/Sucrose buffer, provided ready to use, depending on the ISVD tested).

At the start of the experiments (h: 0) appropriate animals were engrafted with 15×10⁶ in vitro expanded human T cells (Texp) from three individual donors (A, B and C) via intravenous injection (IV).

### Administration of Therapy

All treatments (vehicle, T028200179, T028200150 and T028200057) were administered to the appropriate animals via a single intravenous (bolus) injection on day 0, 2 hours after T cells were implanted (h:2). at 1 mg/kg (5 ml/kg). Constructs were given every three days for a total of 6 administrations and the experiment ended on day 29.

To follow the human T cell engraftment, blood samples were collected during the experiment on days 3, 10, 14, 17 and 21 or at individual end timepoints for flow cytometry analysis of T cell populations.

### Measurement of Tumor Growth

Tumor sizes were measured using a digital caliper (S_Cal EVO Bluetooth, Switzerland) on the day of randomization and then twice weekly. Tumor volumes were calculated according to the formula Tumor volume (TV)=(l×w²)×0.5, where l=largest diameter and w=width (perpendicular diameter) of the tumor (in mm). Group median TV values were used for drawing growth curves and for treatment evaluation for as long as more than 50% of the animals in a group remained alive. Animals were weighed twice a week, or daily if body weight loss more than 10% was recorded. Mice with tumors larger than 2000 mm³, ulcerated tumors, body weight loss >30%, continuous body weight loss >20% or any severe impairment of general health conditions were euthanized.

### Statistical Analysis

For statistical analysis purposes, tumor volume changes from baseline in the mouse efficacy study were compared to a vehicle control group using a two-way analysis of variance (Anova-type) with the factors treatment and day (repeated) and their interactions, followed by a contrast analysis with Bonferroni-Holm adjustment for multiplicity. The statistical analyses were performed using SAS 9.4 for Windows 10. Values of P<0.05 were considered significant.

### Results

The protease activated ISVD construct T028200057 induced significant tumor growth inhibition and tumor regression. The tumor regression is not observed with the active (i.e., non masked) T028200179 administrated to the mice at the same dose level (FIG. 19).

### 7. EMBODIMENTS

The present technology is further represented by the below embodiments.
Embodiment 1. Polypeptide, comprising
   a) a first immunoglobulin single variable domain (ISVD) specifically binding to human serum albumin, wherein said ISVD essentially consists of 4 framework regions (FR1 to FR4 respectively) and 3 complementarity determining regions (CDR1 to CDR3 respectively), wherein
      the amino acid sequence of CDR1 (according to Abm) is GFTFRSFGMS (SEQ ID NO: 29), or an amino acid sequence with 4, 3, 2 or 1 amino acid differences with the sequence GFTFRSFGMS;
      the amino acid sequence of CDR2 (according to Abm) is SISGSGSDTL (SEQ ID NO: 30), or an amino acid sequence with 4, 3, 2 or 1 amino acid differences with the sequence SISGSGSDTL; and
      the amino acid sequence of CDR3 (according to Abm) is GGSLSR, or an amino acid sequence with 4, 3, 2 or 1 amino acid differences with the sequence GGSLSR (SEQ ID NO: 31);
   b) a second ISVD specifically binding to the constant domain of a human and non-human primate T cell receptor (TCR) present on a T cell, wherein said ISVD essentially consists of 4 framework regions (FR1 to FR4 respectively) and 3 complementarity determining regions (CDR1 to CDR3 respectively), wherein
      the amino acid sequence of CDR1 (according to Abm) is WDVHKINFYG (SEQ ID NO: 5), or an amino acid sequence with 4, 3, 2 or 1 amino acid differences with the sequence WDVHKINFYG;
      the amino acid sequence of CDR2 (according to Abm) is HISIGDQTD (SEQ ID NO: 3), or an amino acid sequence with 4, 3, 2 or 1 amino acid differences with the sequence HISIGDQTD; and
      the amino acid sequence of CDR3 (according to Abm) is LSRIWPYDY (SEQ ID NO: 6), or an amino acid sequence with 4, 3, 2 or 1 amino acid differences with the sequence LSRIWPYDY; and
   c) a targeting moiety that specifically binds a target antigen on a target cell, wherein said target antigen is not TCR or serum albumin, and wherein said target cell is not a T cell,
wherein the first and second ISVD are linked via a linker that is susceptible to cleavage by a protease, and
wherein the first ISVD is a C-terminal or N-terminal ISVD.
Embodiment 2. Polypeptide according to Embodiment 1, wherein the second ISVD has a CDR1 with amino acid sequence WDVHKINFYG (SEQ ID NO: 5), or an amino acid sequence with 2 or 1 amino acid differences with the sequence WDVHKINFYG, wherein the amino acid differences are selected from:
   W to G
   D to Y.
Embodiment 3. Polypeptide according to Embodiment 1 or 2, wherein the second ISVD has a CDR3 with amino acid sequence LSRIWPYDY (SEQ ID NO: 6), or an amino acid sequence with 1 amino acid difference with the sequence LSRIWPYDY, wherein the amino acid difference is selected from:
   W to Y.
Embodiment 4. Polypeptide according to any one of Embodiments 1-3, wherein the second ISVD has a CDR1 with amino acid sequence WDVHKINFYG (SEQ ID NO: 5), a CDR2 with amino acid sequence HISIGDQTD (SEQ ID NO: 3), and a CDR3 with amino acid sequence LSRIWPYDY (SEQ ID NO: 6).
Embodiment 5. Polypeptide according to any one of Embodiments 1-4, wherein the first ISVD has a CDR1 with amino acid sequence GFTFRSFGMS (SEQ ID NO: 29), a CDR2 with amino acid sequence SISGSGSDTL (SEQ ID NO: 30), and a CDR3 with amino acid sequence GGSLSR (SEQ ID NO: 31).
Embodiment 6. Polypeptide according to any one of Embodiments 1-5, wherein the first ISVD and/or the second ISVD is a heavy-chain ISVD.
Embodiment 7. Polypeptide according to any one of Embodiments 1-6, wherein the first ISVD and/or the second ISVD is selected from a VHH, a humanized VHH, a (single) domain antibody, a dAb, and a camelized VH.
Embodiment 8. Polypeptide according to any one of Embodiments 1-7, wherein the first ISVD has at least 80%, preferably at least 90%, more preferably at least 95% sequence identity with the amino acid sequence of SEQ ID NO: 32, in which for the purposes of determining the degree of sequence identity, the amino acid residues that form the CDR sequences are disregarded.
Embodiment 9. Polypeptide according to any one of Embodiments 1-8, wherein the amino acid sequence of the first ISVD comprises or consists of SEQ ID NO: 32.
Embodiment 10. Polypeptide according to any one of Embodiments 1-9, wherein the second ISVD has at least 80%, preferably at least 90%, more preferably at least 95% sequence identity with the amino acid sequence of SEQ ID NO: 1, in which for the purposes of determining the degree of sequence identity, the amino acid residues that form the CDR sequences are disregarded.
Embodiment 11. Polypeptide according to any one of Embodiments 1-10, wherein the amino acid sequence of the second ISVD comprises or consists of SEQ ID NO: 1.
Embodiment 12. Polypeptide according to any one of Embodiments 1-11, wherein the targeting moiety is an ISVD.
Embodiment 13. Polypeptide according to any one of Embodiments 1-12, wherein the targeting moiety is selected from a VHH, a humanized VHH, a (single) domain antibody, a dAb, and a camelized VH.
Embodiment 14. Polypeptide according to any one of Embodiments 1-13, wherein the targeting moiety specifically binds a tumor associated antigen.
Embodiment 15. Polypeptide according to any one of Embodiments 1-14, wherein the targeting moiety specifically binds a tumor antigen.
Embodiment 16. Polypeptide according to any one of Embodiments 1-15, wherein cleavage of the linker by a protease results in an activated polypeptide and, wherein the activated polypeptide induces T cell activation (as measured by flow cytometry) which increases by at least 20-fold, preferably at least 40-fold, more preferably at least 70-fold, most preferably at least 80-fold after cleavage of the linker by the protease compared to the polypeptide wherein the linker has not been cleaved by the protease.
Embodiment 17. Polypeptide according to any one of Embodiments 1-16, wherein the polypeptide induces T cell activation (as measured by flow cytometry) only after cleavage of the linker by a protease.
Embodiment 18. Polypeptide according to any one of Embodiments 1-17, wherein cleavage of the linker by a protease results in an activated polypeptide and wherein the activated polypeptide induces T cell activation with an EC50 value of at most about 10⁻⁹ M, at most about 5.10⁻¹⁰ M, preferably at most about 10⁻¹⁰ M, as determined by the CD69 expression on primary T cells measured in flow cytometry.
Embodiment 19. Polypeptide according to any one of Embodiments 1-18, wherein cleavage of the linker by a protease results in an activated polypeptide and wherein the activated polypeptide induces T cell activation with an EC50 value of at most about 10^{31 10} M, preferably at most about 10⁻¹¹ M, as determined by the CD69 expression on PBMCs measured in flow cytometry.
Embodiment 20. Polypeptide according to any one of Embodiments 1-19, wherein cleavage of the linker by a protease results in an activated polypeptide and wherein the activated polypeptide induces T cell activation (as determined by the CD69 expression on PBMCs measured in flow cytometry) that increases by at least 40-fold, by at least 100 fold, preferably by at least 1000-fold after cleavage of the linker by a protease compared to the polypeptide wherein the linker has not been cleaved by the protease.
Embodiment 21. Polypeptide according to any one of Embodiments 1-20, wherein cleavage of the linker by a protease results in an activated polypeptide and wherein the activated polypeptide has an affinity (KD) for binding TCR (as measured by surface plasmon resonance) of at most about 10⁻⁷ M, preferably of at most about 5·10⁻⁸ M.
Embodiment 22. Polypeptide according to any one of Embodiments 1-21, wherein cleavage of the linker by a protease results in an activated polypeptide and wherein the activated polypeptide has an affinity (KD) for TCR (as measured by surface plasmon resonance) that increases by at least 4-fold after cleavage of the linker by the protease compared to the polypeptide wherein the linker has not been cleaved by the protease.
Embodiment 23. Polypeptide according to any one of Embodiments 1-22, wherein cleavage of the linker by a protease results in an activated polypeptide and wherein the activated polypeptide induces T cell mediated cytotoxicity (as measured by an impedance-based cytotoxicity assay) that increases by at least 40-fold, preferably at least 100-fold, more preferably at least 300-fold, most preferably at least 1000-fold after cleavage of the linker by the protease compared to the polypeptide wherein the linker has not been cleaved by the protease.
Embodiment 24. Polypeptide according to any one of Embodiments 1-23, wherein cleavage of the linker by a protease results in an activated polypeptide and wherein the activated polypeptide induces T cell mediated cytotoxicity (as measured by an impedance-based cytotoxicity assay) with an IC50 value of at most about 5·10⁻⁹ M, at most about 10⁻⁹ M, preferably at most about 10⁻¹⁰ M, more preferably at most about 10⁻¹¹ M, most preferably at most about 5·10⁻¹² M.
Embodiment 25. Polypeptide according to any one of Embodiments 1-24, wherein cleavage of the linker by a protease results in an activated polypeptide and wherein the activated polypeptide induces PBMC mediated cell toxicity (as measured by an impedance-based cytotoxicity assay) that increases by at least 40-fold, preferably at least 100-fold, more preferably at least 300-fold, most preferably at least 1000-fold after cleavage of the linker by the protease compared to the polypeptide wherein the linker has not been cleaved by the protease.
Embodiment 26. Polypeptide according to any one of Embodiments 1-25, wherein cleavage of the linker by a protease results in an activated polypeptide and wherein the activated polypeptide induces PBMC mediated cell toxicity (as measured by an impedance-based cytotoxicity assay) with an IC50 value of at most about 10⁻⁸ M, at most about 10⁻⁹ M, preferably at most about 10⁻¹⁰ M, more preferably at most about 10⁻¹¹ M, most preferably at most about 10⁻¹² M.
Embodiment 27. Polypeptide according to any one of Embodiments 1-26, wherein the polypeptide induces cytokine secretion (as measured by a bead-based multiplex assay) upon cleavage of the protease cleavable linker.
Embodiment 28. Polypeptide according to any one of Embodiment 1-27, wherein cleavage of the linker by a protease results in an activated polypeptide and, wherein the activated polypeptide induces secretion of IL-2 (as measured in a bead-based multiplex assay), with an EC50 value of at most about 10-8 M, at most about 10-9 M, more preferably at most about 10-10 M, most preferably at most about 10-11 M.
Embodiment 29. Polypeptide according to any one of Embodiments 1-28, wherein cleavage of the linker by a protease results in an activated polypeptide and wherein the polypeptide induces secretion of IL-6 that increases by at least 10-fold, preferably at least 100-fold after cleavage of the linker by the protease compared to the polypeptide wherein the linker has not been cleaved by the protease.
Embodiment 30. Polypeptide according to any one of Embodiments 1-29, wherein cleavage of the linker by a protease results in an activated polypeptide and wherein the activated polypeptide induces secretion of IFN-γ with an EC50 value of at most about 10⁻⁹ M, at most about 10⁻⁹ M, more preferably at most about 10⁻¹⁰ M, most preferably at most about 5·10⁻¹¹ M.
Embodiment 31. Polypeptide according to any one of Embodiments 1-30, wherein cleavage of the linker by a protease results in an activated polypeptide and wherein the polypeptide induces secretion of IFN-γ that increases by at least 10-fold, preferably at least 50-fold, more preferably at least 100-fold after cleavage of the linker by the protease compared to the polypeptide wherein the linker has not been cleaved by the protease.
Embodiment 32. Polypeptide according to any one of Embodiments 1-31, wherein cleavage of the linker by a protease results in an activated polypeptide and wherein the activated polypeptide induces secretion of TNF-α with an EC50 value of at most about 5.10⁻⁹ M, at most about 10⁻⁹ M, more preferably at most about 10⁻¹⁰ M, most preferably at most about 5.10⁻¹¹ M.
Embodiment 33. Polypeptide according to any one of Embodiments 1-32, wherein cleavage of the linker by a protease results in an activated polypeptide and wherein the polypeptide induces secretion of TNF-α that increases by at least 10-fold, preferably at least 50-fold, more preferably at least 100-fold after cleavage of the linker by the protease compared to the polypeptide wherein the linker has not been cleaved by the protease.
Embodiment 34. Polypeptide according to any one of Embodiments 1-33, wherein cleavage of the linker by a protease results in an activated polypeptide and wherein the activated polypeptide induces secretion of IL-2 with an EC50 value of at most about 10⁻⁸ M, at most about 10⁻⁹ M, more preferably at most about 10⁻¹⁰ M, most preferably at most about 5.10⁻¹¹ M.
Embodiment 35. Polypeptide according to any one of Embodiments 1-34, wherein cleavage of the linker by a protease results in an activated polypeptide and wherein the polypeptide induces secretion of IL-2 that increases by at least 3-fold, at least 10-fold, preferably at least 100-fold after cleavage of the linker by the protease compared to the polypeptide wherein the linker has not been cleaved by the protease.
Embodiment 36. Polypeptide according to any one of Embodiments 1-35, wherein the protease cleavable linker is cleaved by a protease selected from enterokinase (EK), urokinase (uPA), prostate specific antigen (PSA), and matriptase.
Embodiment 37. Polypeptide according to any one of Embodiments 1-36, wherein the protease cleavable linker has an amino acid sequence that is selected from SEQ ID NOs: 52-55.
Embodiment 38. Composition comprising the polypeptide according to any one of Embodiments 1-37.
Embodiment 39. Composition according to Embodiment 38, wherein the composition is a pharmaceutical composition, further comprising an acceptable pharmaceutical carrier, diluent or excipient and/or adjuvant.
Embodiment 40. Polypeptide according to any one of Embodiments 1-37 or composition according to Embodiment 38 or 39 for use as a medicament.
Embodiment 41. Polypeptide according to any one of Embodiments 1-37 or composition according to Embodiment 38 or 39 for use in the prevention, treatment or amelioration of a disease selected from the group consisting of a proliferative disease, an inflammatory disease, an infectious disease and an autoimmune disease.
Embodiment 42. Polypeptide or composition according to Embodiment 41, wherein the disease is cancer.
Embodiment 43. Method of producing a polypeptide according to any one of Embodiments 1-37, comprising the steps of
   a. expressing in a suitable host cell or host organism or in another suitable expression system, a nucleic acid sequence encoding the polypeptide; optionally followed by
   b. isolating and/or purifying the polypeptide.
Embodiment 44. Nucleic acid encoding the polypeptide according to any one of Embodiments 1-37.
Embodiment 45. Vector comprising a nucleic acid according to Embodiment 44.
Embodiment 46. Non-human host or host cell expressing the polypeptide according to any one of Embodiments 1-37, and/or comprising the nucleic acid according to Embodiment 44 or the vector according to Embodiment 45.

1. A polypeptide, comprising:
a) a first immunoglobulin single variable domain (ISVD) specifically binding to human serum albumin, wherein said ISVD essentially consists of 4 framework regions (FR1 to FR4 respectively) and 3 complementarity determining regions (CDR1 to CDR3 respectively), wherein:
the amino acid sequence of CDR1 is GFTFRSFGMS (SEQ ID NO: 29), or an amino acid sequence with 4, 3, 2 or 1 amino acid differences with the sequence GFTFRSFGMS (SEQ ID NO: 29);
the amino acid sequence of CDR2 is SISGSGSDTL (SEQ ID NO: 30), or an amino acid sequence with 4, 3, 2 or 1 amino acid differences with the sequence SISGSGSDTL (SEQ ID NO: 30); and
the amino acid sequence of CDR3 is GGSLSR (SEQ ID NO:31), or an amino acid sequence with 4, 3, 2 or 1 amino acid differences with the sequence GGSLSR (SEQ ID NO: 31);
b) a second ISVD specifically binding to the constant domain of a human and non-human primate T cell receptor (TCR), wherein said ISVD essentially consists of 4 framework regions (FR1 to FR4 respectively) and 3 complementarity determining regions (CDR1 to CDR3 respectively), wherein:
the amino acid sequence of CDR1 is WDVHKINFYG (SEQ ID NO: 5), or an amino acid sequence with 4, 3, 2 or 1 amino acid differences with the sequence WDVHKINFYG (SEQ ID NO: 5);
the amino acid sequence of CDR2 is HISIGDQTD (SEQ ID NO: 3), or an amino acid sequence with 4, 3, 2 or 1 amino acid differences with the sequence HISIGDQTD (SEQ ID NO: 3); and
the amino acid sequence of CDR3 is LSRIWPYDY (SEQ ID NO: 6), or an amino acid sequence with 4, 3, 2 or 1 amino acid differences with the sequence LSRIWPYDY (SEQ ID NO: 6); and
c) a targeting moiety that specifically binds a target antigen on a target cell, wherein said target antigen is not TCR or serum albumin, and wherein said target cell is not a T cell,
wherein the first and second ISVD are linked via a linker that is susceptible to cleavage by a protease,
wherein the first ISVD is a C-terminal or N-terminal ISVD, and
wherein the CDRs of the first ISVD and the CDRs of the second ISVD are determined according to Abm.

2. The polypeptide according to claim 1, wherein the second ISVD has:
a CDR1 with amino acid sequence WDVHKINFYG (SEQ ID NO: 5), or an amino acid sequence with 2 or 1 amino acid differences with the sequence WDVHKINFYG (SEQ ID NO: 5), wherein the amino acid differences are selected from: W to G; or D to Y; and/or
a CDR3 with amino acid sequence LSRIWPYDY (SEQ ID NO: 6) or an amino acid sequence with 1 amino acid difference with the sequence LSRIWPYDY (SEQ ID NO: 6), wherein the amino acid difference is: W to Y.

3. (canceled)

4. The polypeptide according to claim 1, wherein:
the second ISVD has a CDR1 with amino acid sequence WDVHKINFYG (SEQ ID NO: 5), a CDR2 with amino acid sequence HISIGDQTD (SEQ ID NO: 3), and a CDR3 with amino acid sequence LSRIWPYDY (SEQ ID NO: 6); and/or
the first ISVD has a CDR1 with amino acid sequence GFTFRSFGMS (SEQ ID NO: 29), a CDR2 with amino acid sequence SISGSGSDTL (SEQ ID NO: 30), and a CDR3 with amino acid sequence GGSLSR (SEQ ID NO: 31).

5. (canceled)

6. The polypeptide according to claim 1, wherein the first ISVD and/or the second ISVD is a heavy-chain ISVD, optionally wherein the first ISVD and/or the second ISVD is selected from a VHH, a humanized VHH, a domain antibody, a dAb, and a camelized VH.

7. (canceled)

8. The polypeptide according to claim 1, wherein:
the first ISVD has at least 80% sequence identity with the amino acid sequence of SEQ ID NO: 32, in which for the purposes of determining the degree of sequence identity, the amino acid residues that form the CDR sequences are disregarded; and/or
the second ISVD has at least 80% sequence identity with the amino acid sequence of SEQ ID NO: 1, in which for the purposes of determining the degree of sequence identity, the amino acid residues that form the CDR sequences are disregarded.

9. The polypeptide according to claim 1, wherein:
the amino acid sequence of the first ISVD comprises or consists of SEQ ID NO: 32: and/or
the amino acid sequence of the second ISVD comprises or consists of SEQ ID NO: 1.

10.-11. (canceled)

12. The polypeptide according to claim 1, wherein the targeting moiety is an ISVD, optionally wherein the targeting moiety:
is selected from a VHH, a humanized VHH, a (single) domain antibody, a dAb, and a camelized VH;
specifically binds a tumor associated antigen; and/or
specifically binds a tumor antigen.

13.-15. (canceled)

16. The polypeptide according to claim 1, wherein cleavage of the linker by a protease results in an activated polypeptide and, wherein, compared to the polypeptide wherein the linker has not been cleaved, the activated polypeptide induces T cell activation:
which increases by at least 20-fold;
with an EC50 value of at most about 10⁻⁹ M, as determined by the CD69 expression on primary T cells; and/or
with an EC50 value of at most about 10⁻¹⁰ M, as determined by the CD69 expression on PMBCs.

17.-20. (canceled)

21. The polypeptide according to claim 1, wherein cleavage of the linker by a protease results in an activated polypeptide and wherein the activated polypeptide has an affinity (K_{D}) for binding TCR:
of at most about 10⁻⁷ M; and/or
that increases by at least 4-fold after cleavage of the linker by the protease compared to the polypeptide wherein the linker has not been cleaved by the protease.

22. (canceled)

23. The polypeptide according to claim 1, wherein cleavage of the linker by a protease results in an activated polypeptide and wherein the activated polypeptide induces T cell mediated cytotoxicity:
that increases by at least 40-fold after cleavage of the linker by the protease compared to the polypeptide wherein the linker has not been cleaved by the protease; and/or
with an IC50 value of at most about 5·10⁻⁹ M.

24. (canceled)

25. The polypeptide according to claim 1, wherein cleavage of the linker by a protease results in an activated polypeptide and wherein the activated polypeptide induces PBMC mediated cell toxicity:
that increases by at least 40-fold after cleavage of the linker by the protease compared to the polypeptide wherein the linker has not been cleaved by the protease; and/or
with an IC50 value of at most about 10⁻⁸ M.

26. (canceled)

27. The polypeptide according to claim 1, wherein the polypeptide induces cytokine secretion upon cleavage of the protease cleavable linker, optionally wherein cleavage of the linker by a protease results in an activated polypeptide and wherein, compared to the polypeptide wherein the linker has not been cleaved by the protease, the activated polypeptide induces secretion of:
IL-6 with an EC50 value of at most about 10⁻⁸ M and/or that increases by at least 10-fold; and/or
IFN-γ with an EC50 value of at most about 10⁻⁹ M and/or that increases by at least 10-fold; and/or
TNF-α with an EC50 value of at most about 5·10⁻⁹ M and/or that increases by at least 10-fold; and/or
IL-2 with an EC50 value of at most about 10⁻⁸ M and/or that increases by at least 3-fold.

28.-35. (canceled)

36. The polypeptide according to claim 1, wherein the protease cleavable linker is cleaved by a protease selected from enterokinase (EK), urokinase (uPA), prostate specific antigen (PSA), and matriptase.

37. The polypeptide according to claim 1, wherein the protease cleavable linker has an amino acid sequence that is selected from SEQ ID NOs: 52-55.

38. A composition comprising the polypeptide according to claim 1, optionally wherein the composition is a pharmaceutical composition, further comprising an acceptable pharmaceutical carrier, diluent or excipient, and/or adjuvant.

39.-40. (canceled)

41. A method for the treatment or amelioration of a proliferative disease, an inflammatory disease, an infectious disease, or an autoimmune disease, said method comprising administering to a subject in need thereof a pharmaceutically effective amount of the polypeptide according to claim 1, optionally wherein the proliferative disease is cancer.

42. (canceled)

43. A method of producing a polypeptide according to claim 1, comprising the steps of
a. expressing in a suitable host cell or host organism or in another suitable expression system, a nucleic acid sequence encoding the polypeptide; optionally followed by
b. isolating and/or purifying the polypeptide.

44. A nucleic acid encoding the polypeptide according to claim 1.

45. A vector comprising a nucleic acid according to claim 44.

46. A non-human host or host cell expressing the polypeptide according to claim 1.
